(19) United States
(12) Patent Application Publication
(10) Pub. No.: US 2025/0384971 A1
(43) Pub. Date: Dec. 18, 2025

(54) METHODS FOR CHARACTERISATION OF NANOCARRIERS

(71) Applicant: NUNTIUS THERAPEUTICS LIMITED, London (GB)

(72) Inventors: Tristan HENSER-BROWNHILL, London (GB); Albert KWOK, London (GB); Liam Thomas MARTIN, London (GB); Benita NAGEL, London (GB)

(21) Appl. No.: 19/108,111

(22) PCT Filed: Sep. 1, 2023
(86) PCT No.: PCT/EP2023/074085

§ 371 Date: Feb. 28, 2025

(30) Foreign Application Priority Data
Sep. 2, 2022 (GB) 2212806.0

(51) Int. Cl. G16C 60/00; B82Y 5/00; G16C 20/70

(52) U.S. Cl. CPC G16C 60/00; B82Y 5/00; G16C 20/70

(57) ABSTRACT

This invention provides methods for predicting functional and/or structural properties of a nanocarrier that is a non-viral delivery system, along with related methods, systems and products. Functional properties of a nanocarrier may include transfection efficiency, structural properties of a nanocarrier characterise physico-chemical properties such as polydispersity, size and zeta potential. Methods include computational modelling such as machine learning and related statistical techniques.

### FIELD OF THE INVENTION

The present invention relates to methods of predicting properties of nanocarriers for cellular payload delivery, methods of nanocarrier engineering using predictions of nanocarrier properties, and related methods and products.

### BACKGROUND

Nucleic acid therapeutics have the potential to be the next generation of precision medicines which will transform healthcare. However, key challenges remain. One such challenge is the efficient and safe delivery of the nucleic acid therapies to patients. Current viral and non-viral vector platforms often fall at the clinical translation stage due to off-target effects, immune activation and difficulty manufacturing said vectors at scale.

In the context of in vivo delivery of nucleic acids, viral-derived vectors have been studied extensively and some are very advanced in the clinic, including adeno-associated viruses (AAVs) (Sheridan et al, 2011 and Wang et al, 2019). The use of these systems are however limited to delivering DNA of <5 kb and cannot transport RNA or larger DNA. Despite the advances in these delivery systems, there is also still the potential for random insertions and immunotoxicity resulting from the use of these systems. In particular, when targeting non-liver tissues, which requires the use of higher doses, the AAV systems can be highly immunogenic. This tendency to generate an immune response to the AAV system also limits the usefulness of the system for repeat dosing as patients typically develop immunity to the AAV delivery system. Finally, AAV delivery systems are known to be expensive and difficult to manufacture at scales required for therapeutic use and at Good Manufacturing Practice (GMP) grade.

Non-viral vector systems for nucleic acid delivery to cells and tissues in vivo are also being investigated. One such system is the use of nanoparticles or nanocarriers comprising a payload and a carrier component. For example, lipid nanoparticles (LNPs) have been used to encapsulate and deliver mRNA, for example in COVID-19 vaccines (Qui et al, 2021). Indeed, to address the difficulty of getting plasmid DNA and mRNA to traverse the plasma membrane it can be useful to encapsulate the nucleic acids and neutralise the charge for effective delivery. However, the LNPs currently used in the clinic are well suited to transfect a small number of muscle and immune cells local to the site of delivery, or to target the liver when administered intravenously. They are less suitable for targeting other tissues, which makes them less suitable for targeting diseases other than those associated with the liver. To overcome the various drawbacks of the above delivery systems, the addition of peptides to lipid based vectors has been investigated. In these hybrid systems, the peptides and lipids associate with the nucleic acid to form nanoparticles that can be internalised by cells. The peptide element may be linear (Kwok et al, 2016) or branched, such as a peptide dendrimer (Kwok et al, 2013). In WO 2022/162200, it was recently demonstrated by the inventors that delivery of long nucleic acids, such as mRNA, can be achieved in vivo using peptide dendrimer/lipid hybrid systems.

Regardless of the type of nanocarriers used, the number of possibilities for every element of the formulation of a nanocarrier means that the design space is extremely large. This is additionally very poorly understood as the functional properties of a nanocarrier may be influenced by a great variety of factors. Further, the costs associated with formulating and testing diverse nanocarriers (considering the size of the design space) is often prohibitive.

### SUMMARY OF THE INVENTION

The present inventors postulated that it may be possible to train machine learning models to predict functional and structural properties of nanocarriers in silico, in order to facilitate the design of nanocarriers and prioritise candidates for testing. They collected a data set of structural properties and transfection efficiency for a complex type of nanocarrier (comprising a peptide dendrimer/branched peptide, lipid and payload), and showed that even in this complex system they were able to reliably predict transfection efficiency based solely on features of the nanocarrier that can be calculated from a candidate formulation. While there have been previous attempts to characterise nanocarrier formulations using machine learning, most prior approaches were limited to predicting properties that are tied to a particular payload and where the prediction uses properties of the payload. For example, Gao, Haoshi, et al. (‘Development of in silico methodology for siRNA lipid nanoparticle formulations.’ Chemical Engineering Journal, 442, (2022)) described a method to predict the knockdown efficiency of siRNA carrying LNPs in vitro using features including the siRNA sequence. By contrast, the present inventors were able to predict transfection efficiency (independently of payload) based on features of the nanocarrier itself. This provides a powerful tool to design nanocarriers usable for a variety of applications.

Thus, in a first aspect, this invention provides a method of predicting one or more properties of a nanocarrier, wherein the nanocarrier is a non-viral cell delivery system, the method comprising: determining the value of one or more structural properties and/or functional properties of the nanocarrier; and predicting the values of one or more properties of the nanocarrier by providing the determined value(s) as input to a machine learning model that has been trained to take as input the values of one or more input structural properties of a nanocarrier and produce as output the values of one or more output functional properties and optionally one or more output structural properties of a nanocarrier different from the input structural properties of the nanocarrier; wherein structural properties of a nanocarrier characterise physico-chemical properties of the nanocarrier and are independent of the activity of a nanocarrier payload. Advantageously, the predicted one or more functional properties of the nanocarrier may comprise the transfection efficiency.

Indeed, the present inventors have identified that it was possible to predict at least the transfection efficiency of a nanocarrier using machine learning models trained using physico-chemical properties of the nanocarrier, enabling characterisation of the nanocarrier for a variety of uses not limited to a particular payload.

As the skilled person understands, the complexity of the operations described herein (due at least to the complexity of performing the calculations as described herein, and the amount of data that is typically associated with training and use of machine learning models (including e.g. artificial neural networks and random forest models), are such that they are beyond the reach of a mental activity. Thus, unless context indicates otherwise (e.g. where sample preparation or acquisition steps are described), all steps of the methods described herein are computer implemented.

The method may have any one or more of the following optional features.

The nanocarrier may be a lipid-based nanoparticle, a peptide-containing nanoparticle, or a peptide containing lipid nanoparticle. A lipid-based nanoparticle may be a lipoplex, a lipid nanoparticle or a peptide containing lipid nanoparticle. The nanoparticle may be a peptide dendrimer/lipid hybrid nanoparticle. Thus, the nanocarrier may comprise a lipid component and/or a peptide component. The peptide component may comprise a branched peptide such as a peptide dendrimer. The nanocarrier may comprise a nucleic acid payload.

The input properties may comprise or consist of input structural properties. The input structural properties of the nanocarrier may comprise or consist of properties that are quantified in silico. The output structural properties of the nanocarrier may be (i.e. consist of) properties that are experimentally determined. The input properties may comprise input functional properties and the output properties may comprise functional structural properties. The input functional properties of the nanocarrier may comprise or consist of properties that are quantified in vitro. The output functional properties of the nanocarrier may comprise or consist of properties that are determined in vivo. Properties that are experimentally determined are properties that cannot be theoretically calculated accurately, and are therefore typically measured. Measurements of structural properties are typically performed in vitro but may in some circumstances be performed in vivo. As the skilled person understands, the properties are experimentally determined in training data used to train the model, but are predicted by the model. Properties that are quantified in silico may be theoretically calculated (e.g. molecular weight, number of a particular amino acid, hydrophobicity, etc.) or may be predicted from other structural properties using a machine learning model as described herein. For example, structural features such as size or PDI may not be theoretically calculated accurately but may be predictable using a machine learning model as described herein, using other structural features as described herein that are calculated theoretically. These predictions may be used as input to a machine learning model trained to predict functional properties including transfection efficiency. The input structural and/or functional features may comprise properties that are experimentally determined. For example, the size and/or PDI of a nanocarrier may be measured and the measurements may be used to predict an output functional property as described herein. As another example, an input functional property such as in vitro transfection efficiency may be used to predict an output functional property such as in vitro transfection efficiency. As yet another example, an input functional property such as cell specific transfection efficiency may be used to predict an output functional property such as tissue targeting. This may still be advantageous even though it requires obtaining and testing the nanocarrier, because testing structural properties is typically less onerous than testing functional properties, and some functional properties are significantly easier to test than others.

The determined structural properties of the nanocarrier may be selected from global nanocarrier structural properties and component specific structural properties. Component-specific properties may be selected from lipid-specific properties and peptide-specific properties. The determined global nanocarrier specific structural properties may be selected from: a protein to payload ratio, a lipid to payload ratio, a size-related metric, and a charge-related metric. A protein to payload ratio may be a N/P ratio. A lipid to payload ratio may be a L ratio. A lipid to payload ratio may be a w/w ratio. A lipid to payload ratio may be a molar ratio. A charge-related metric may be a zeta potential. A size-related metric may be the hydrodynamic size or the polydispersity index. The determined global nanocarrier specific structural properties may advantageously include the protein to payload ratio and/or the lipid to payload ratio. The component specific structural properties may be selected from: lipid-specific properties, peptide-specific properties, and learned features derived from local structural properties, wherein learned features are features identified by a trained machine learning model from a multidimensional input, wherein the local structural properties are structural properties of individual chemical entities within a component. The individual chemical entities may be atoms, lipid chains or amino acids. Lipid-specific properties may be selected from: lipid identity, lipid type, ratio of different lipids or lipid types, length of lipid chains, lipid melting point, lipid saturation, and molecular weight. Peptide specific properties may be selected from molecular weight, charge, mass to charge ratio, scores indicative of the hydrophilicity and/or hydrophobicity of peptides or amino acids, extinction coefficient, isoelectric point, sequence length, presence of specific residues in the sequence, absence of specific residues in the sequence, number of specific residues in the sequence, proportion of specific residues in the sequence, number of branch points in a branched peptide, number of generations in a branched peptide, the number of amino acids in a generation of a branched peptide, and absorbance at a particular wavelength (e.g. 205 nm or 280 nm). Peptide specific properties may apply to a peptide component of the nanocarrier and/or to a peptide payload.

The input structural properties of the nanocarrier may comprise any one or more of: the protein to payload ratio, the lipid to payload ratio, the number of positively charged sidechains in a peptide component or peptide payload of the nanocarrier, the number of negatively charged sidechains in a peptide component or peptide payload of the nanocarrier, the number of polar sidechains of the amino acids of a peptide component or peptide payload of the nanocarrier, the number of ionisable sidechains of the amino acids of a peptide component or peptide payload of the nanocarrier, the number of generations in a branched dendrimer component of the nanocarrier, the number of amino acids in a generation of a branched peptide component of the nanocarrier, the molecular weight of any component of the nanocarrier, the molecular weight of a peptide component of the nanocarrier, the number of a particular amino acid in a peptide component or peptide payload of the nanocarrier, the number of His residues in a peptide component or peptide payload of the nanocarrier, a score indicative of the hydrophilicity of residues in a peptide component or peptide payload of the nanocarrier, the percentage or proportion of hydrophobic residues in a peptide component or peptide payload of the nanocarrier, the percentage or proportion of hydrophilic residues in a peptide component or peptide payload of the nanocarrier, the presence of a particular type of residues in a particular region of a peptide component or peptide payload of the nanocarrier, the absorbance of a peptide component of the nanocarrier at a predetermined wavelength (e.g. 205 nm or 280 nm), the net charge of the peptide component of the nanocarrier at a particular pH (e.g. 7.4, 6.5, 5.5, 4.5, or any value between 7.4 and 4.5), the isoelectric point of the peptide component of the nanocarrier, and the isoelectric point of a particular region of the peptide component of the nanocarrier (e.g. isoelectric point considering only amino acids in the first, second and/or third generation of a dendrimer peptide component, isoelectric point considering only amino acid in the core and/or outermost generation of a dendrimer peptide component). The input structural properties of the nanocarrier may comprise one or more of, or all of the properties listed in Table 1 or Table 2. The input structural properties of the nanocarrier may comprise at least one of: the protein to payload ratio (e.g. N/P ratio), the number of positively charged sidechains in a peptide component of the nanocarrier, the molecular weight of a peptide component, and a value indicative of the hydrophilicity of the peptide component. The input structural properties of the nanocarrier may include at least the protein to payload ratio (e.g. N/P ratio). The input structural properties of the nanocarrier may comprise at least two of: the protein to payload ratio (e.g. N/P ratio), the number of positively charged sidechains in a peptide component of the nanocarrier, the molecular weight of a peptide component, and a value indicative of the hydrophilicity of the peptide component. The input structural properties of the nanocarrier may comprise at least one of: the to payload ratio (e.g. N/P ratio), the number of positively charged sidechains in a peptide component of the nanocarrier, the molecular weight of a peptide component, and a value indicative of the hydrophilicity or hydrophobicity of the peptide component. The input structural properties of the nanocarrier may comprise at least two of: the protein to payload ratio (e.g. N/P ratio), a charge related metric (e.g. the number of positively charged sidechains in a peptide component of the nanocarrier and/or the total number of histidines in a peptide component of the nanocarrier and/or the total number of charges in a peptide component of the nanocarrier), the molecular weight of a peptide component, the lipid ratio and a value indicative of the hydrophilicity or hydrophobicity of the peptide component (e.g. the sum of the Hopp-Woods hydrophilicity scores from each residue in the peptide component, the sum of the Hopp-Woods hydrophilicity scores from hydrophobic residues in the peptide component and/or the percentage of hydrophobic residues in the peptide component).

The input structural and/or functional properties of the nanocarrier may be normalised values. The output functional and/or structural properties of the nanocarrier may be normalised values. A normalised value may be obtained by dividing a determined by a maximum possible or observed value and/or by subtracting a determined value by a minimum possible or observed value. The method may further comprise normalising the values of one or more input structural features by dividing a determined value a maximum possible or observed value and/or by subtracting a determined value by a minimum possible or observed value. A maximum/minimum observed value may be the maximum/minimum observed value of a property determined in a training data set. A maximum/minimum possible value may be the maximum/minimum theoretically possible value for a property.

The output nanocarrier structural properties may be selected from: size of the nanocarrier, polydispersity index of the nanocarrier, and zeta potential of the nanocarrier. The predicted one or more functional properties of the nanocarrier may further comprise one or more properties selected from: cell-specific payload, tissue specific payload delivery, in vitro cytotoxicity, in vivo cytotoxicity, in vitro immunogenicity, in vivo immunogenicity, nanocarrier temperature dependent structural stability, nanocarrier pH dependent structural stability, nanocarrier pH dependent transfection efficiency, nanocarrier concentration dependent structural stability, and nanocarrier pH dependent transfection efficiency, nanocarrier time dependent structural stability, nanocarrier structural stability in serum. The input functional properties of the nanocarrier may be selected from: cell specific payload delivery/transfection, transfection efficiency (e.g. in vitro transfection efficiency), cytotoxicity (e.g. in vitro cytotoxicity), immunogenicity (e.g. in vitro immunogenicity), nanocarrier temperature dependent structural stability, nanocarrier pH dependent structural stability, nanocarrier pH dependent transfection efficiency, nanocarrier concentration dependent structural stability, nanocarrier time dependent structural stability, nanocarrier structural stability in serum, and nanocarrier pH dependent transfection efficiency. Inputs functional properties may be functional properties that are measured in vitro. For the avoidance of doubt, any input structural properties are different from any output structural properties, and any input functional properties are different from any output functional properties,

The machine learning model may have been trained using training data comprising the value of the one or more input structural properties of a plurality of nanocarriers and the value of one or more functional properties and optionally one or more output structural properties of said plurality of nanocarriers. The training data may comprise data for at least 10, at least 25, at least 50, at least 100 different nanocarriers, or at least 150 different nanocarriers. The different nanocarriers may differ from each other by at least one of: The composition of a lipid component, the composition of a protein component, the protein to payload ratio and the lipid to payload ratio. The machine learning model may have been trained using training data comprising data for a plurality of nanocarriers of the same type as the nanocarrier for which the one or more properties are predicted. The nanocarriers for which the one or more properties are predicted and the plurality of nanocarriers in the training data may be lipid-based nanoparticles, peptide-lipid hybrid nanoparticles or dendrimer peptide-lipid hybrid nanoparticles. The machine learning may have been trained using training data comprising measured transfection efficiency for a plurality of nanocarriers. The transfection efficiency may be a normalised transfection efficiency. The transfection efficiency may be measured using a reporter gene signal, such as e.g. by measuring fluorescence associated with expression of a fluorescent protein, or expression of a luciferase protein. The transfection efficiency in the training data may be expressed in fluorescence units associated with expression of a genetic payload encoding a fluorescent protein. The transfection efficiency may be normalised using a positive and/or negative control value. The transfection efficiency may be an in vitro transfection efficiency. The transfection efficiency may be an in vivo transfection efficiency. The transfection efficiency may have been measured using one or more cell lines and/or types of cells. The machine learning may have been trained using training data comprising measured in vitro transfection efficiency for a plurality of nanocarriers, and the predicted transfection efficiency may be indicative of in vitro and optionally in vivo transfection efficiency. The present inventors have surprisingly discovered that machine learning models trained based on data comprising in vitro transfection efficiency are able to provide an output that is indicative of in vivo transfection efficiency. The machine learning may have been trained using training data comprising measured transfection efficiency for a plurality of nanocarriers in one or more cell lines, and the predicted transfection efficiency may be indicative of transfection efficiency in one or more cell lines comprised in the training data and/or in one or more cell lines not comprised in the training data. The present inventors have surprisingly discovered that machine learning models trained based on data comprising transfection efficiency measured in one cell line are able to provide an output that is predictive of transfection efficiency in other cell lines, i.e. that the predictions generalise to most cell lines and/or that the machine learning models can be trained to provide predictions for different cell lines.

The machine learning model may be a non-linear model. The machine learning model may be an artificial neural network or a tree-based model such as a random forest model or gradient boosted tree. The machine learning model may comprise a plurality of models, wherein each model of the plurality of models has been trained to predict a different set of one or more functional and/or structural properties of a nanocarrier. The machine learning model may comprise a model that has been trained to jointly predict a plurality of functional and/or structural properties of a nanocarrier. The machine learning model may comprise an ensemble of models and the one or more functional and/or structural properties of the nanocarrier may be obtained by combining the output of the models in the ensemble of models (e.g. by obtaining an average or other summary statistic over the predictions of the models in the ensemble). The present inventors have identified that non-linear models perform better at capturing relationships between structural features of the nanocarriers and functional properties (likely because of non-linearities in these relationships). The machine learning model may comprise an ANN with a single output node for each predicted feature and a softplus activation function after each output node corresponding to an unbounded feature (e.g. size, transfection performance) and/or a sigmoid activation function after each output node corresponding to a value bounded between 0 and 1 (e.g. PDI). The machine learning model may comprise a neural network trained using a grid search and cross-validation to identify one or more parameters of the neural network architecture, such as the number of hidden layers and/or the number of nodes in one or more hidden layers. The machine learning model may comprise a neural network trained using dropout, optionally with a 50% dropout rate.

In a second aspect, the invention provides a method of providing a tool for predicting one or more properties of a nanocarrier, wherein the nanocarrier is a non-viral cell delivery system, the method comprising: obtaining a training data set comprising, for each of a plurality of nanocarriers: experimental data quantifying one or more functional properties of the nanocarrier; experimental and/or in silico determined values of one or more structural properties of the nanocarrier; and training a machine learning model to predict the values of one or more functional properties and optionally one or more experimentally determined structural properties of a nanocarrier using input values comprising one or more structural properties of the nanocarrier and/or one or more functional properties of the nanocarrier, optionally comprising at least the in silico determined values of one or more structural properties of the nanocarrier; wherein structural properties of a nanocarrier characterise physico-chemical properties of the nanocarrier and are independent of the activity of a nanocarrier payload. The one or more functional properties of the nanocarrier may comprise the transfection efficiency.

The method of the present aspect may have any of the features described in relation to the previous aspect. The method may have any one or more of the following optional features. Obtaining the training data set may comprise obtaining the plurality of nanocarriers and/or measuring the one or more functional properties of the plurality of nanocarriers and/or measuring or determining the values of the one or more structural properties of the nanocarrier. Obtaining the training data set may comprise normalising each of the determined properties using control values and/or minimum and/or maximum observed or possible values in the training data set.

The method may further comprise obtaining for each nanocarrier the values of learned features derived from local structural properties, using a trained machine learning model (such as e.g. a CNN trained on unrelated data), wherein the local structural properties are structural properties of individual chemical entities within a component, optionally wherein the individual chemical entities are atoms, lipid chains or amino acids. The method may further comprise predicting the value of one or more properties of one or more candidate nanocarriers using the methods of any embodiment of the first aspect.

The methods of any aspect described herein may further comprise outputting a result of the method, for example through a user interface.

The methods of any aspect may further comprise one or more of: predicting the one or more properties for a further one or more nanocarriers, selecting one or more of a plurality of nanocarriers using the predicted one or more properties of said plurality of nanocarriers, obtaining the nanocarrier or a selected one or more of the nanocarriers, testing one or more properties of the nanocarrier or a selected one or more of the nanocarriers, formulating a composition comprising the nanocarrier or a selected one or more of the nanocarriers, testing one or more properties of a composition comprising the nanocarrier or a selected one or more of the nanocarriers.

The present invention also relates to use of the methods as described herein in the engineering of a nanocarrier with one or more desired properties.

Thus, in a third aspect, the invention provides a method of prioritising testing of nanocarriers for cellular payload delivery, the method comprising: predicting the properties of a plurality of candidate nanocarriers using the method of any embodiment of the first aspect and selecting a candidate nanocarrier from the plurality of candidate nanocarriers for testing on the basis of the predicted value of the one or more functional and/or structural properties.

In a fourth aspect, the invention provides a method of designing a nanocarrier for cellular payload delivery, the method comprising: obtaining a plurality of candidate nanocarriers, predicting the properties of a plurality of candidate nanocarriers using the method of any embodiment of the first aspect and selecting a candidate nanocarrier from the plurality of candidate nanocarriers on the basis of the predicted value of the one or more functional and/or structural properties.

According to a fifth aspect, there is provided a method of providing a candidate nanocarrier with a predetermined one or more properties, the method comprising: providing a plurality of candidate nanocarriers, wherein the candidate nanocarriers differs from each other in their composition and/or structure; predicting the value of one or more functional and/or structural properties of the candidate nanocarriers using the method of any embodiment of the first aspect; and selecting a candidate nanocarrier from the plurality of candidate nanocarriers on the basis of the predicted value of the one or more functional and/or structural properties. Selecting a candidate nanocarrier may comprise ranking the plurality of candidate nanocarriers based on at least one of the predicted one or more functional and/or structural properties. The one or more functional and/or structural properties may comprise the transfection efficiency. The method may comprise ranking the plurality of candidate nanocarriers based on their predicted transfection efficiency. Selecting a candidate nanocarrier may comprise excluding nanocarriers of the plurality of candidate nanocarriers that have a predicted value of one or more functional and/or structural properties that does not meet one or more predetermined criteria. Selecting a candidate nanocarrier may comprise selecting nanocarriers of the plurality of candidate nanocarriers that have a predicted value of one or more functional and/or structural properties that meet one or more predetermined criteria. For example, the method may comprise excluding candidate nanocarriers that have a predicted size above a predetermined value. As another example, the method may comprise excluding candidate nanocarriers that have a predicted PDI above a predetermined value, such as e.g. 0.35, 0.30, 0.25, or 0.20. The method may further comprise formulating and/or experimentally validating.

The method may further comprise optimising one or more selected candidate nanocarriers. The method may further comprise preselecting a plurality of candidate nanocarriers (e.g. prior to predicting the nanocarrier properties) based on expert knowledge and/or random modification of previously obtained nanocarriers.

According to a sixth aspect, there is provided a system comprising: a processor; and a computer readable medium comprising instructions that, when executed by the processor, cause the processor to perform the steps of the method of any embodiment of any preceding aspect. The system may further comprise one or more automated laboratory equipment, for example to perform the steps of obtaining and/or testing candidate nanocarriers.

According to a seventh aspect, there is provided one or more non-transitory computer readable medium or media comprising instructions that, when executed by one or more processors, cause the one or more processors to perform the steps of the method of any embodiment of any of the first to fifth aspects.

According to a further aspect, there is provided a computer program comprising code which, when the code is executed on a computer, causes the computer to perform the steps of any method described herein.

The invention includes the combination of the aspects and preferred features described except where such a combination is clearly impermissible or expressly avoided.

### BRIEF DESCRIPTION OF THE FIGURES

Embodiments and experiments illustrating the principles of the invention will now be discussed with reference to the accompanying figures in which:

FIG. 1 shows a flow diagram illustrating, in schematic form, a method for predicting one or more properties of a nanocarrier and/or selecting or providing a nanocarrier according to the disclosure.

FIG. 2 shows a flow diagram illustrating, in schematic form, a method for providing a tool for predicting one or more properties of a nanocarrier and/or for selecting a nanocarrier according to the disclosure.

FIG. 3 shows an embodiment of a system for predicting one or more properties of a nanocarrier and/or selecting or providing a nanocarrier according to the disclosure.

FIG. 4 shows schematically a process used to provide a tool for predicting properties of nanocarriers and for designing nanocarriers according to an exemplary embodiment.

FIG. 5 illustrates schematically the formulation of a nanocarrier according to the present disclosure, comprising a plurality of elements that can be combined in a modular fashion, each of which can be associated alone or in combination with other elements with one or more features characteristics that can be used to predict one or more properties of the nanocarrier.

FIG. 6 illustrates schematically the configuration of a third generation dendrimer represented diagrammatically (with N-termini on the left and C-terminus on the right). The circle represents the core sequence. Each triangle represents a branching residue, such as lysine. Each rectangle represents a peptide motif. There are two peptide motifs in the first layer, four peptide motifs in the second layer, and eight peptide motifs in the third layer of the third generation dendrimer. A corresponding second generation dendrimer would lack the third layer of eight peptide motifs. A first generation dendrimer would lack the third layer of eight peptide motifs and the second layer of four peptide motifs as well as the layer of branching residues between the third and second layer.

FIG. 7 shows the replicability of transfection efficiency experiments for a primary control (CTL) and 3 well characterised (VAL1, VAL2, VAL3) nanocarriers. Error bars are 95% confidence intervals.

FIG. 8 shows the kernel density estimation of distributions of structural features nanocarriers sizes, PDI and functional feature transfection efficiency used as ground truth for predicted features (A), and the kernel density estimation of distributions of structural features used as predictive features (B), in a training data set comprising 165 peptide dendrimer nanocarriers. See Table 1 for features legend.

FIG. 9 is a correlation matrix of min-max scaled features considered for prediction of properties of nanocarriers (properties described in Table 1).

FIG. 10 shows examples of visualisations of 2D feature encodings for a first, second and third generation dendrimer (respectively from left to right), encoding the molecular weight (top row) and the Hopp-Woods hydrophilicity (bottom row).

FIG. 11 illustrates schematically the architecture of an artificial neural network (ANN) usable according to an embodiment of the disclosure.

FIG. 12 shows the 10-fold cross validation learning curves for ANNs trained to predict respective properties of nanocarriers (from left to right: transfection efficiency, size, PDI). The dotted vertical line marks the best epoch, the black line is the training curve and the grey line is the validation curve (average MSE loss±standard error).

FIG. 13 illustrates schematically the architecture of an convolutional neural network (CNN) usable according to an embodiment of the disclosure. GAP-global average pooling.

FIG. 14 shows the 10-fold cross validation learning curves for CNNs trained to predict respective properties of nanocarriers (from left to right: transfection efficiency, size, PDI). The dotted vertical line marks the best epoch, the black line is the training curve and the grey line is the validation curve (average MSE loss±standard error).

FIG. 15 shows the 10-fold cross validation overall performance comparison of various models used to predict properties of nanocarriers (from left to right: transfection efficiency, size, PDI). RC=random choice, MLR=multiple linear regression, RF=random forest.

FIG. 16 shows kernel density estimation of distributions of nanocarriers sizes, PDI and transfection efficiency in a training data set comprising 165 peptide dendrimer nanocarriers, overlaid with the KDE distribution for predictions from RF models trained with leave-one-out-cross validation (LOOCV). The overall MAE for the models are also overlaid on the graphs.

FIG. 17 shows results of a validation study for a method of the disclosure. (A) a bar chart showing the transfection performance, size and PDI observed and predicted (MAE) using random forest models according to the disclosure, for each of 7 nanocarriers (E1, E2, E3, E4, R1, R2, R3) at each of 3 NP ratios (A: NP=0.6, B: NP=4 and C: NP=8)-filtered to exclude nanocarriers with predicted PDI>=0.25; the error bars are the standard deviation around the MAE across trees in the forest; (B) same as A but including all nanocarriers and ordering nanocarriers by increasing measured PDI; (C) box plots of measured transfection performance of a training data set used in the examples vs a machine learning selected dataset (all<0.25 pdl). The bars are the inter-quartile range (IQR) and line in the box is the median. Any dots above and below are more than 1.5*IQR. Mann-Whitney U test showed the difference is highly significant p<0.001.

FIG. 18 shows the results of a permutation importance analysis for a plurality of RF modes for prediction of the transfection performance, size and PDI. Each bar shows the increase in error (MAE) as a respective feature is randomly shuffled prior to model validation.

FIG. 19 shows the results of validation of a plurality of RF models trained for prediction of the transfection performance, size and PDI using different subsets of predictive features. Each bar shows the error (MAE) for the respective models (10-fold cross-validation, bars show the average with error bars showing the standard deviation). The legend of the features is as in Table 1.

FIG. 20 shows results of classification accuracy for prediction of nanocarriers as good PDI samples (PDI<0.3) vs poor PDI samples (PDI≥0.3). A: Bar plot showing the proportion of measured or predicted

(LOOCV) good PDI samples (PDI<0.3) versus poor quality PDI examples for the training set (n=165); top right shows an associated confusion matrix for classification of good or poor PDI samples in the training set. B: Equivalent bar plot for the real-world test set (n=21) and associated confusion matrix in the top right. Error bars are 95% confidence.

FIG. 21 shows ROC (receiver operating characteristic) curves for logistic regression and a random forest classifier used to classify nanocarriers between: (A) a positive class with PDI<0.3 and a negative class with PDI>=0.3, and (B) a positive class with PDI<0.2 and a negative class with PDI>=0.2. AUC is also shown as a comparative metric.

FIG. 22 shows scatter plots showing the correlation between ground truth (y) and predicted (9) zeta potential values for both linear regression and a random forest. The MAE and R2 are reported for comparative purposes.

FIG. 23 shows a simple schematic of a neural network architecture used in an example of the disclosure. Here X are the input features 1 to n, and y is the predicted property (e.g. transfection performance). The softplus log (exp (x)+1) output function enables smoother training by ensuring that the output is always positive, as it is impossible to have a negative value of the particular property predicted (transfection ratio).

FIG. 24 is a schematic diagram showing the application of Monte Carlo Dropout (Gal and Ghahramani, 2016) to nanocarrier attribute predictions (9). Here connections in the trained network are disabled at random before a prediction of nanocarrier performance is made. After multiple iterations, the predictions are aggregated, and the mean taken as the final result. This can improve performance, and also permits the estimation of confidence in any prediction by analysing the variance across iterations.

FIG. 25 shows results of evaluation of exemplary models of the disclosure on an independent held-out test set. Left shows a random forest model, right shows a neural network. Error bars are standard deviations indicating model confidence in each prediction. For the random forest, these are calculated from either 512 individual decision tree predictions. For the neural network these are calculated from 329 Monte Carlo Dropout iterations. The coefficient of determination (R2) and line of best fit between the ground truth and predicted values is also shown.

FIG. 26 shows results of a permutation importance analyses from either random forest or neural network modelling for transfection prediction. This uses the same data as FIG. 25 (see Example 10). Each bar shows the increase in error (MAE) as a respective feature is randomly shuffled prior to validation. Error bars show standard error.

### DETAILED DESCRIPTION OF THE INVENTION

Aspects and embodiments of the present invention will now be discussed with reference to the accompanying figures and the technical definitions that follow below. Further aspects and embodiments will be apparent to those skilled in the art. All documents mentioned in this text are incorporated herein by reference.

### Nanocarriers

As used herein, a “nanocarrier” refers to a nanoparticle that is configured to deliver a payload, such as e.g. a genetic payload, into a cell. As used herein, a nanocarrier is a non-viral delivery system. Nanocarriers as described herein may be lipid-based nanocarriers (such as e.g. lipoplexes and lipid nanoparticles). For example, a nanocarrier may be a dendrimer-lipid based delivery system. Examples of such systems are described in WO 2022/162200. A nanocarrier may be a polymer or peptide-based delivery system.

### Payload/Cargo

Nanocarriers (also referred to herein as “nanoparticles” comprise a cargo (also referred to a payload) to be delivered into cells. The cargo may be a nucleic acid cargo (also referred to herein as “genetic cargo”). The nucleic acid may be DNA and/or RNA. The invention is not limited in principle to any particular types of nucleic acids. The characteristics of the nucleic acid may depend on the particular use for the nanocarrier. Exemplary uses are described below, including e.g. the delivery of mRNA, long non coding RNA, miRNA, siRNA, antisense oligonucleotides (ASOs) etc. to a cell or cells. Delivery can be in vivo or in vitro.

The cargo may be a drug (e.g. an active small molecule). The drug may be hydrophilic and encapsulated within a lipid bilayer of a lipid-based nanoparticle. Instead or in addition to this, the drug may be hydrophobic and reside within a lipid bilayer of a lipid-based nanoparticle. The cargo may be a chemotherapeutic agent.

The cargo may be a protein or sets of proteins, such as e.g. one or more recombinant proteins.

### Lipid-Based Nanoparticles

In some embodiments, the nanoparticle is a lipid nanoparticle (LNP). The LNP may contain different types of lipids such as an ionisable lipid, a cationic lipid, an anionic lipid, a helper lipid, a phospholipid (for example, phosphatidylcholine and phosphatidylethanolamine), and a polyethylene glycol (PEG)-functionalized lipid, and cholesterol. Further description of the LNP can be found in the review (Hou et al. 2021). The lipid-based nanoparticle may be a lipoplex or a lipid nanoparticle which contains lipids selected from: cationic lipids, anionic lipids, ionisable lipids, helper lipids and neutral lipids.

In embodiments, the nanoparticle is a lipid-based nanoparticle. Thus, in embodiments, the nanoparticle comprises one or more lipids. The nanoparticle may be a liposome (a vesicle comprising at least one lipid bilayer), such as a lipoplex (a nanoparticle comprising a cationic lipid and nucleic acid cargo), or a more complex lipid-based nanoparticle such as e.g. a nanoparticle comprising a lipid component, a peptide component and a cargo. The lipid of the nanoparticle may comprise a cationic lipid, a neutral lipid, an anionic lipid and/or an ionisable lipid. In some embodiments, the lipid of the composition comprises a saturated fatty acid. Additionally or alternatively, the lipid of the composition may comprise an unsaturated fatty acid. In some embodiments, the lipid comprises 1, 2, 3, 4, 5 or 6 fatty acid chains. Preferably, the lipid comprises 2, 3, 4 or 6 fatty acid chains.

In some embodiments, the lipid comprises dioleoylphosphatidylethanolamine (DOPE) and/or N-[1-(2,3-dioleyloxy) propyl]-N,N, N-trimethylammonium chloride (DOTMA). In some embodiments, the lipid comprises dioleoylphosphatidylethanolamine (DOPE) and dioleoylphosphatidylglycerol (DOPG). The lipid component of the composition may comprise DOTMA, DOPE, DOPC and/or DOPG. The lipid based nucleic acid delivery system may be DOTMA/DOPE.

The amount of lipid component can be expressed in a weight:weight ratio (“w/w”, or “w:w”), with respect to the amount of the nucleic acid in the composition. For example, the w/w ratio may be in the range of 1:50 to 50:1. As another example, the amount of lipid (by weight) may be 1:1 to 50:1, or 2:1 to 25:1 with respect to the amount of nucleic acid (by weight). The lipid:nucleic acid ratio can be at least 2:1. The weight:weight ratio of lipid:nucleic acid may be about 10:1 to 25:1. These ratios refer to the weight of the total lipid. As described herein, the composition may comprise a lipid component that includes more than one lipid, e.g. a mixture of two, three or four lipids. The weight of the lipid component is the total (combined) weight of these lipid components. In embodiments, each lipid component is mixed in approximately equal proportions. The amount of lipid can be expressed in a molar ratio with respect to the amount of the nucleic acid in the composition.

### Peptide Dendrimers

In embodiments, the nanocarrier is a nanoparticle comprising a branched peptide component, such as a peptide dendrimer component, a nucleic acid cargo component and a lipid component. The nucleic acid cargo may comprise one or more different nucleic acids. The peptide dendrimer component may comprise one or more different peptide dendrimers. The lipid component may comprise one or more different lipids.

A dendrimer may be a first, second or third generation peptide dendrimers, meaning that the dendrimers have up to three ‘layers’ of peptide motifs interspersed between ‘branching’ residues, such as lysine. First generation dendrimers have the following structure, shown in the N-termini to C-terminus orientation, and taking Lys to be the branching unit: (N-term-Pep1) 2-Lys-(Core)-(C-term)

Second generation dendrimers have the following structure, shown in the N-termini to C-terminus orientation, and taking Lys to be the branching unit: (N-term-Pep2) 4-Lys2-(Pep1) 2-Lys-(Core)-(C-term)

Third generation dendrimers have the following structure, shown in the N-termini to C-terminus orientation, and taking Lys to be the branching unit: (N-term-Pep3) 8-Lys4-(Pep2) 4-Lys2-(Pep1) 2-Lys-(Core)-(C-term)

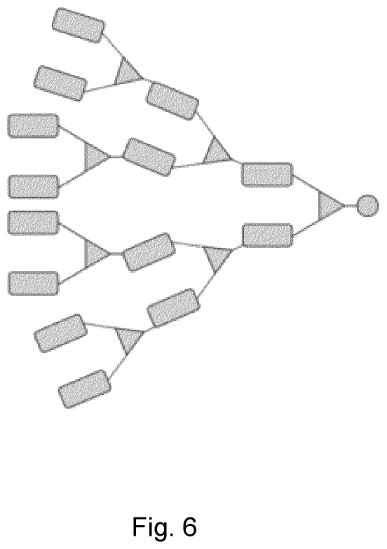

Third generation dendrimers are represented diagrammatically (with N-termini on the left and C-terminus on the right) in FIG. 6.

In FIG. 6, the circle represents the core sequence. Each triangle represents a branching residue, such as lysine. Each rectangle represents a peptide motif. There are two peptide motifs in the first layer, four peptide motifs in the second layer, and eight peptide motifs in the third layer of the third generation dendrimer. The N- and C-termini may be derivatised with further chemical motifs, as discussed herein. For instance, while in un-derivatised embodiments, the C-terminus is a carboxylic acid, in other embodiments the C-terminus is derivatised e.g. to comprise a primary amide group, CONH2 (instead of COOH), as a result of the chemical pathway used to synthesise the dendrimer. Functionally important derivatisations such as antibodies, peptide groups, sugar groups and/or lipid chains are also envisaged, which can be attached to the N- and/or C-termini, or at other positions along the dendrimer. The N-terminus of the peptide dendrimers disclosed herein may be acetylated.

Unless specified otherwise, dendrimers as used herein can be first, second or third generation. This can be defined structurally as follows: First generation dendrimers comprise a core peptide sequence, a first branching residue and two first peptide motifs each attached to the first branching residue. The two first peptide motifs independently consist of a single amino acid, dipeptide, tripeptide or tetrapeptide motifs. Second generation dendrimers further comprise two second branching residues (e.g. lysine) and four second peptide motifs, wherein one of the second branching residues is covalently bound to one of the first peptide motifs and the other second branching residue is covalently bound to the other first peptide motif, and wherein each second branching residue is covalently bound to two second peptide motifs. The four second peptide motifs independently consist of a single amino acid, dipeptide, tripeptide or tetrapeptide motifs. Third generation dendrimers further comprises four third branching residues (e.g. lysine) and eight third peptide motifs, wherein each second peptide motif is respectively covalently bound to one of the third branching residues such that each third branching residue is covalently bound to one second peptide motif, and wherein each third branching residue is covalently bound to two third peptide motifs. The eight third peptide motifs independently consist of a single amino acid, dipeptide, tripeptide or tetrapeptide motifs. Each of the first, second and third peptide motifs, where present, may comprise (1) an amino acid with a basic side chain such as, but not limited to, Lysine (K) or Arginine (R) or Histidine (H), (2) an amino acid with an acidic side chain such as but not limiting to Aspartic acid (D) and Glutamic acid (E), (3) an amino acid with a non-polar side chain such as, but not limited, to Glycine (G), Alanine (A), Valine (V), Isoleucine (I), Leucine (L), Methionine (M),Phenylalanine (F), Beta-alanine (B), Tryptophan (W), Proline (P), aminohexanoic acid (X or Acp) and Cysteine (C) and (4) an amino acid with a uncharged polar side chain such as, but not limited to, Asparagine (N), Glutamine (Q), Serine(S), Threonine (T) and Tyrosine (Y).

Examples of dendrimers are provided in WO 2022/162200, the entire content of which is incorporated herein by reference. For instance, in dendrimers where each peptide motif is an Arg-Leu (RL) dipeptide, this structure can be denoted G1-RL, G1,2-RL and G1,2,3-RL. In dendrimers where each peptide motif is a Lys-Leu (KL) dipeptide, this structure is denoted G1-KL, G1,2-KL and G1,2,3-KL. In dendrimers where each peptide motif is a Leu-Arg (LR) dipeptide, this structure is denoted G1-LR, G1,2-LR and G1,2,3-LR.

‘G1’. ‘G2’ and ‘G3’ refer to the ‘generation-1’, ‘generation-2’ and ‘generation-3’ peptide motifs of the first, second and third layers, respectively. Each amino acid residue can be an L-amino acid or a D-amino acid. D-amino acids may be designated using lower case letters in the single-letter code. Alternatively, dendrimers in which each amino acid is the D-isoform can be written with a preceding “D-” before the short-form denotation of the dendrimer.

Peptide dendrimers with a specified core are also discussed herein. For example, in dendrimers where a defined peptide core is intended, for example a peptide core of Arg-His-Cys, this structure can be denoted as RHCG1, 2-RL. It will be understood that per the nomenclature of the peptide dendrimers disclosed here, in the previous example, ‘G’ refers to the ‘generation’ of the peptide motif and not a Glycine residue. In contrast, a peptide dendrimer with a structure denoted as GSCG1, 2-RL, 3-LR it will be understood that the first ‘G’ in this context will refer to a Glycine residue while the second ‘G’ refers to the ‘generation’ of the peptide motifs. In other examples, where a defined core is intended, the core sequence will be underlined leaving the “G” denoting the generation not underlined (e.g. GSCG1, 2-RL, 3-LR).

Each peptide motif of a dendrimer may individually comprise naturally occurring L- or D-amino acids and/or non-naturally occurring L- or D-amino acids, for example, Beta-alanine (B) or aminohexanoic acid (X or Acp).

A peptide dendrimer may further comprises a cell penetrating peptide. The cell penetrating peptide may comprises a TAT derived sequence. The cell penetrating peptide may comprise the peptide sequence XRXRRBRRXRRBRXB, where X is 6-aminohexanoic acid and B is beta-alanine.

A peptide dendrimer may further comprise an alkyl chain, alkenyl chain, an antibody or a fragment thereof, a sugar, and/or a fatty acid. An alkyl or alkenyl chain may be conjugated to the core peptide sequence, for instance at the C terminus of the peptide dendrimer. Alternatively, the alkyl or alkenyl chain may be conjugated to the N terminus of the peptide dendrimer.

In some embodiments, alkyl or alkenyl chains comprise from about 5 carbons to about 50 carbons, preferably from about 12 to about 30 carbons. A peptide dendrimer may comprise a fatty acid conjugated to the C terminus of the peptide dendrimer. In other embodiments, the peptide dendrimer comprises a fatty acid conjugated to the N terminus of the peptide dendrimer.

A peptide dendrimer may comprise a cell penetrating peptide, an endosomal escape peptide, a nuclear localisation motif, and/or a fatty acid. The cell penetrating peptide, endosomal escape peptide, nuclear localisation motif, and/or fatty acid may be conjugated to the C-terminus of the peptide dendrimer. Alternatively, or in addition to, the cell penetrating peptide, endosomal escape peptide, nuclear localisation motif, and/or fatty acid may be conjugated to the N-terminus of the first and/or second peptide dendrimer.

### Structural Properties of Nanocarriers

The present disclosure relates at least in part to the use of structural properties of nanocarriers as predictive or predicted features of machine learning models. Structural properties of a nanocarrier characterise physico-chemical properties of the nanocarrier. Structural properties of a nanocarrier are preferably independent of the activity of a nanocarrier payload. For example, where the payload is a genetic payload encoding a siRNA, a structural property of the nanocarrier does not depend on the level of inhibition achieved by the payload once delivered in a cell. As another example, where the payload is a drug, a structural property of the nanocarrier does not depend on the IC50 of the drug when delivered in a cell. The structural properties of the nanocarrier may characterise the nanocarrier itself, independently of the exact identity of the payload. Structural properties of the nanocarrier may depend on the type of payload as well as the amount of payload. For example, when the payload is a nucleic acid, the structural properties of the nanocarrier may be independent of the sequence of the nucleic acid but may depend on the amount of nucleic acid in the nanoparticle and/or on the type of nucleic acid (such as e.g. single stranded RNA such as mRNA or double-stranded DNA such as plasmid DNA). Structural properties of the nanocarrier may characterise physico-chemical properties of the nanocarrier as a whole, and/or on physico-chemical properties of any component of the nanocarrier (including but not limited to the payload, a lipid component, a peptide component, etc.).

Structural properties of a nanocarrier may characterise any one or more of: the size of the nanocarrier, the distribution of size of the nanocarriers (e.g. polydispersity index, PDI), the identity of a component of the nanocarrier (other than the payload), the amount or relative amounts of one or more components of the nanocarrier (including the amount or relative amount of payload, such as e.g. the amount of a component of the nanocarrier relative to the amount of payload), one or more physico-chemical properties of one or more components of the nanocarrier. Physico-chemical properties of one or more components of a nanocarrier may be selected from: molecular weight, charge, mass to charge ratio, hydrophilicity or hydrophobicity scores (e.g. as described in Hopp-Woods, 1981 or Kyte-Doolittle, 1982), extinction coefficient, isoelectric point, sequence length (e.g. for a peptide component of a nanocarrier), presence/absence of specific residues in the sequence (e.g. for a peptide component of a nanocarrier), number of branch point or generations in branched peptides.

The structural properties of a nanocarrier can be classified into: global nanocarrier features (also referred to as ‘nanocarrier-specific features’ or ‘nanocarrier features’), and component specific features. Nanocarrier features are features that characterise the nanocarrier as whole, including features that integrate the properties of more than one component of the nanocarrier. By contrast, component-specific features characterise a particular component of the nanocarrier. Thus, the particular structural properties that may apply to a particular type of nanocarrier typically depends on the composition of the nanocarrier. For example, a nanocarrier that is a lipid-based nanoparticle may have component-specific features that characterise the lipid component of the nanocarrier.

Global nanocarrier features may be selected from: the protein to payload ratio (also referred to as “peptide to payload ratio” for a nanocarrier comprising a peptide component, with units depending on the particular payload considered, in the particular example of a nucleic acid cargo this may be quantified as a N/P ratio as further explained below), the lipid to payload ratio (for a nanocarrier that is a lipid-based nanoparticle), size (e.g. in nm), polydispersity index (PDI), charge (e.g. zeta potential). A protein to payload ratio may quantify the ratio of amino acid containing components (e.g. peptides, proteins, peptide dendrimers) to payload components (e.g. nucleic acids). The lipid to payload ratio may be referred to as “L ratio” and may be a w:w ratio or a molar ratio. The protein to payload ratio is the ratio of peptide or protein component to payload component.

Component-specific features may be features of the lipid component of a lipid-based nanoparticle. For example, a lipid-specific feature may be selected from: the type of lipid or combinations of lipid types, the ratio(s) of different lipids or lipid types (in terms of e.g. weight or number of molecules/molar or weight concentration in the nanoparticle), the hydrophobicity, length of chain, or any other parameters characterising the structure or physico-chemical properties of the lipids.

Component-specific features may be peptide specific features, i.e. features of the peptide component of a nanocarrier. Peptide-specific features may be selected from: molecular weight, charge, mass to charge ratio, scores indicative of the hydrophilicity and/or hydrophobicity of peptides or amino acids (e.g. as described in Hopp-Woods, 1981 or Kyte-Doolittle, 1982), extinction coefficient, isoelectric point, sequence length, presence/absence/number/proportion of specific residues or motifs (i.e. combinations of residues) in the sequence or in a particular portion of the sequence (e.g. His, Cys, positively charged residues, negatively charged residues, hydrophobic residues, hydrophilic residues, basic residues, non-polar residues, leucine and/or arginine comprising motifs, etc.), number of branch points and/or generations in branched peptides, number of amino acids in each generation in branched peptides, ratio of different peptides (e.g. by weight or concentration), and absorbance at a predetermined wavelength (e.g. 205 or 280 nm). Any feature of a peptide sequence may be expressed per residue, per subunit of a peptide (e.g. when looking at dendrimers this can be per generation) or as summary values over the entire peptide. A summary value may be a sum, median, mean, or trimmed versions thereof. For example, the presence of cysteine residues in a peptide component indicates that the peptide may form disulphide bonds for example to other peptides in the nanocarrier and/or may be usable to attach targeting sequences. As another example, the presence of histidine residues in a peptide component indicates that the peptide may be charged at lower pH such as e.g. in a cell lysosome. This may contribute to payload escape. Positively charged residues may be residues that have a positively charged sidechain. Positively charged residues may be any residues that are selected from: lysine and arginine. Positively charged residues may exclude lysines where the side chain is not exposed, such as e.g. branching lysines. Hydrophobic residues may be any residues selected from: F, I, L, M, V, W, Y (i.e. Phe, Ile, Leu, Met, Val, Trp, Tyr). Hydrophilic amino acids may be any residues selected from: Arg, Asp, Gln, Asn, Glu, His, Lys, Ser, Thr. Non-polar amino acids may be any amino acids selected from: Ala, Val, Leu, Pro, Met, Try, Gly, Ile, Phe. Basic amino acids may be any amino acids selected from: Arg, Lys, His. Absorbance of a peptide component at 205 nm may be computationally estimated using a method as described in Anthis and Clore (2013). Absorbance of a peptide component at 280 nm may be computationally estimated using a method as described in Gill and von Hippel (1989). Alternatively, absorbance of a peptide component at any wavelength may be measured experimentally. The charge of a peptide component may be a net charge at a predetermined pH. The net charge of a peptide component at a predetermined pH may be computationally estimated from the amino acid composition using:

$$Z = {\sum }_{i} {N}_{i} \frac{1 {0}^{pKai}}{1 {0}^{pH} + 1 {0}^{pKai}} - {\sum }_{j} {N}_{j} \frac{1 {0}^{pH}}{1 {0}^{pH} + 1 {0}^{pKaj}}$$

where Ni are the total quantities of Arginine, Lysine, and Histidine, and the pKai pKa of their side chains and N-termini; and Nj are the total quantities of Aspartic Acid, Glutamic Acid, Cysteine, Tyrosine, and pKaj the pKa of their side chains and C-termini. The predetermined pH may be 7.4, 6.5, 5.5, 4.5 or any experimentally or physiologically relevant pH, including e.g. any pH between 4 and 7.5. The isoelectric point of a peptide component may be the isoelectric point of the entire component or any part thereof. For example, the isoelectric point may be determined for any generation of a peptide dendrimer component (e.g. the first, second, third or outermost generation), for the core of a peptide dendrimer component, or for the entire peptide component. The isoelectric point may be computationally estimated using the net charge formula above, by identifying the pH at which the net charge is closest to 0 amongst a plurality of candidate pH (e.g. evaluating all pH between 0 and 14 with a preset increment, e.g. 0.01).

For prediction of properties for nanocarriers that do not have a particular component, and for which component specific features for that component may therefore be unavailable, a predetermined value may be used (e.g.-1) for these properties.

Component-specific features may be payload specific features, i.e. features of the payload component of a nanocarrier. Payload specific features may be selected from: the amount of payload, the molecular weight of the payload, the ratios of different payloads (e.g. in terms of weight or number of molecules/concentration), the type of nucleic acid payload (e.g. DNA, RNA, double or single stranded), any of the properties listed in relation to peptide specific features in the case of a protein or peptide payload, charge of a drug payload, hydrophobicity of a drug payload, ratio(s), and any structural feature of a drug payload (such as e.g. presence or absence of particular chemical structures or groups).

Component-specific features may be calculated as sums, weighted sums, averages or weighted averages (collectively referred to as “summarised features”) over individual subcomponents such as e.g. where multiple different peptides, cargos or lipids are used. Where weighed sums or averages are used, the weights may be proportional to the relative abundance of the different subcomponents. Instead or in addition to summarised features, component-specific features may be specified individually for each subcomponent, or for one or more of the subcomponents. For example, a machine learning model as described herein may use as input a plurality of features comprising a feature for each of a plurality of subcomponents. The machine learning model may also take as input one or more features that are summarised features for a plurality of subcomponents. Subcomponents refer to a plurality of different molecules that make up a component. For example, subcomponents of a lipid component of a nanocarrier refer to different lipids included in the lipid component of the nanocarrier. Similarly, a payload may comprise a plurality of different nucleic acid molecules or proteins, each representing a subcomponent of the payload component. When a machine learning model takes as input a plurality of features comprising a feature for each of a plurality of subcomponents and/or a summarised feature for a plurality of subcomponents, the machine learning model may be used to predict properties of nanocarriers that do not comprise multiple subcomponents, as well as nanocarriers that comprise subcomponents. For example, features such as ratios of different subcomponents may be set to suitable default value (e.g. 1, 0, etc.). Similarly, features of the single subcomponent may be repeated as input features for individual subcomponents. For example, the machine learning model may have a first input feature that is the molecular weight of a first peptide (subcomponent of a peptide component of a nanocarrier) and a second (respectively third, fourth, etc.) input feature that is the molecular weight of a second (respectively third, fourth, etc.) peptide (subcomponent of a peptide component of a nanocarrier). When using such a machine learning model to predict features of a nanocarrier with a single peptide, the value of the molecular weight for the single peptide may be provided as the value for the first and second (and third, fourth, etc. as the case may be) input feature. An input feature that is the weight ratio of the first peptide to the second peptide may be set to 1 (or any other consistent default value).

Component-specific features may be obtained as learned features (e.g. encodings from a neural network such as a convolutional neural network) extracted from a set of features maintaining the local structural information in the component. For example, in the context of a peptide (or peptide/protein payload), one or more of the features described herein may be specified for each amino acid in the sequence of the peptide, thereby forming a multidimensional feature from which a neural network such as a CNN can learn informative features. For example, features such as molecular weight, Hopp-Woods hydrophilicity and cationic charge may be specified individually for each amino acid in a sequence (optionally min and/or max normalised, such as e.g. using the minimum and maximum value of the respective feature across naturally occurring amino acids). Each property may represent a different channel that is input into the neural network. As another example, in the context of a peptide (or peptide/protein payload) or drug payload, atomic coordinates or any other representation of chemical structure may be used as an input feature to a neural network, the output of which represents learned features of the peptide/protein/drug. A global average pooling operation may be applied to the output of the neural network to obtain a vector of values. Such a vector of values can optionally be concatenated with any additional input features for prediction of the functional and/or structural features of the nanocarrier. Alternatively, a higher dimensional output of the neural network may be provided as an input to the model used to predict the functional and/or structural features of the nanocarrier, alone or in addition to e.g. one or more further features as described herein. A neural network used to obtain learned features may be trained together with a further machine learning model (e.g. a neural network) used to predict functional/structural features using the learned features as input. Alternatively, a neural network used to obtain learned features may be trained separately from the machine learning model (e.g. a neural network) used to predict functional/structural features using the learned features as input. For example, a neural network used to obtain learned features may be trained then the parameters of such a model (e.g. weights of a neural network) may be set (i.e. frozen) and the further machine learning model (e.g. a neural network) used to predict functional/structural features using the learned features as input may be trained (i.e. learning parameters for the further machine learning model but not changing the parameters of the neural network used to obtain learned features).

Depending on the particular embodiment, some structural features may be used as predictive features (inputs to a machine learning model) and/or some structural features may be used as predicted features (outputs of a machine learning model). For example, in the examples below the nanocarrier size (in nm) is used as a predicted feature. However, where a measured or predicted size is available, this can be used as a predictive feature to predict transfection efficiency or another functional parameter or structural feature. As another example, in the examples below the PDI is used a predicted feature. However, where a measured or predicted PDI is available, this can be used as a predictive feature to predict transfection efficiency or another functional parameter or structural feature.

When used as a predictive feature (i.e. as input to a machine learning model), any structural feature of a nanocarrier may be theoretically calculated, measured or predicted. For example, a predictive structural feature may be predicted using a first machine learning model trained to predict the structural feature using as input the value of one or more other structural features. The predictive structural feature may then be used as input to a second machine learning model trained to predict one or more functional features of the nanocarrier (e.g. transfection efficiency). The first and/or second machine learning models may have any of the features described herein. As another example, a predictive structural feature may be predicted using a predetermined empirical relationship, such as e.g. an empirical relationship between amino acid composition of a peptide and hydrophobicity. Any method known in the art for predicting any of the structural features described herein can be used to predict the value of a structural feature for use as input to a machine learning model as described herein. The use of theoretically calculated or predicted structural features as predictive features advantageously does not require to actually obtain the nanocarriers and perform measurements, in order to predict functional and/or further structural properties of the nanocarrier. However, the use of measured structural properties may still be advantageous as many structural properties are significantly easier to measure than functional properties. For example, the size and/or PDI of a nanocarrier is significantly faster, cheaper and easier to determine experimentally than the transfection efficiency or any other functional property of the nanocarrier. Predicted structural features of a nanocarrier may be included as candidate input features alone or in combination with measured structural features of a nanocarrier when training a machine learning model as described herein. This may be used together with a method for assessing the contribution of predictive features to prediction performance of a machine learning model as described herein (e.g. by determining the loss in prediction performance upon permutation/random shuffling of an the values of an input feature in the training dataset). This may be used to determine the importance of any particular input feature in making a prediction, which may in turn be used to determine the one or more input features to be used in a final machine learning model used to make predictions for candidate nanocarriers. When predicted/theoretically calculated and measured features are included as input features, such an approach may be used to quantify the relative importance of predicted/calculated features and measured features, for example to determine whether there is sufficient benefit associated with the use of measured features. This may be used for example to select a predicted structural feature instead of a corresponding measured structural feature (e.g. predicted vs measured size) where the former provides a similar contribution to predictive performance of the machine learning model as the latter.

The N/P ratio (also referred to herein as ‘NP ratio’ or ‘NP’ or ‘N:P ratio’) of a peptide-based nanocarrier refers to the amount of peptide (measured by the number of 1+ charged nitrogen atoms on the peptide, N) to the amount of nucleic acid (measured by the number of 1− charged phosphate groups in the backbone, P) in the nanocarrier. It may be advantageous for this ratio to be greater than 0.05:1, for instance greater than 0.1:1. The N/P ratio terminology can be expressed as “N/P”, “N:P”, or “NP”. In some embodiments, the desired N/P ratio is 0.15:1, or about 0.15:1, or at least 0.15:1. In some embodiments the desired N/P ratio is 0.16:1, or about 0.16:1, or at least 0.16:1. In some embodiments, the desired N/P ratio is at least, or greater than, 1:1, for instance about 2:1 or greater, about 2.5:1 or greater, about 3:1 or greater, about 4:1 or greater, about 5:1 or greater, about 10:1 or up to 20:1. In some embodiments, the N/P ratio is about 5:1, about 8:1, about 10:1, or about 20:1. In some embodiments, the N/P ratio is in the range of about 0.01:1 and 100:1, about 2:1 to about 20:1, or about 2.5:1 to about 10:1. Note that the term “peptide” is used herein in the context of nanocarriers comprising a component that is made of amino acid chains of any size, i.e. not limited to short chains (sometimes referred to as “peptide” by contrast to “proteins” which is sometimes used to refer to longer chains). Thus, the terms “peptide” and “protein” are used interchangeably to refer to components of a nanocarrier that comprise amino acid chains.

The distribution of the size of nanoparticles in a population can be expressed in terms of the population's polydispersity index (PDI). A PDI of 1 indicates a completely polydisperse population of nanoparticles, whereas a PDI of 0 indicates a completely monodisperse population of nanoparticles. Therefore, the PDI can be used as a measure of the uniformity of a population of nanoparticles in a composition. A PDI of 0.35 or less is considered to provide a suitably monodisperse population of nanoparticles for, e.g., development of suitable pharmaceutical compositions. However, for nanoparticles used for in vitro or ex vivo transfection of cells the PDI may be higher than 0.35, for example, the PDI may be equal to or lower than 0.65, 0.6, 0.55, 0.5, 0.45, 0.4, 0.35, or 0.3. A PDI of 0.30, 0.25 or less (e.g. 0.20) may be preferable. PDI<0.2 or ≤0.2 is often used in the literature and widely accepted by chemists as designating good particle quality (Danaei et al. 2018). The present inventors found a threshold of 0.3 to be more useful in practice, and clinically is acceptable (Danaei et al. 2018). Any of the described thresholds may be used depending on the context and desired use. Thus, candidate nanoparticles may be selected according to a method of the disclosure if they have a PDI<=0.35, <=0.30, <=0.25, or <=0.20. Alternatively, candidate nanoparticles may be selected according to a method of the disclosure if they have a PDI<0.35, <0.30, <0.25 or <0.20. Nanoparticles with a predicted PDI<=0.35 and >0.25 may be selected as candidates and their formulation may be adapted to reduce the PDI. The PDI of a particular nanoparticle formulation can be measured by any method standard in the art, but is typically measured using dynamic light scattering (DLS). For example, this may be performed using the Zetasizer Advance Series-Pro according to the manufacturer's instructions. DLS assumes that the particles are spherical, and is therefore a suitable technology to measure size features of any nanocarrier that is expected to be approximately spherical. Nanocarriers that are expected to be approximately spherical include but are not limited to liposomes, lipoplexes, lipid-based nanoparticles (including peptide-lipid hybrid nanoparticles).

A non-uniform or non-monodisperse population of nanoparticles (e.g, wherein the PDI of the nanoparticle is greater than 0.30 or 0.35) may indicate that the binding of a peptide dendrimer to the nucleic acid in a nanoparticle is unstable (i.e. the peptide dendrimer and nucleic acid have a low binding affinity and readily dissociate). In contrast, a uniform or monodisperse population of nanoparticles may indicate that the binding of a peptide dendrimer to a nucleic acid in a nanoparticle is stable (i.e. the peptide dendrimer and nucleic acid bind with high affinity and do not readily dissociate). The PDI of a nanoparticle may influence its suitability for a particular use, as one or more functional properties of the nanoparticle may be influenced by the size of the nanoparticle (such that heterogeneity of size leads to heterogeneity of function). Thus, the PDI of a nanoparticle may be an important characteristic of regulated pharmaceutical products.

The size of a particle may refer to the hydrodynamic size. Hydrodynamic size may be measured using the DLS technique for example using a Zetasizer Advance Series-Pro (Malvern Panalytical Ltd, Malvern, UK) according to the manufacturer's instructions. DLS is a very sensitive, non-invasive method to measure size and size distribution of nanoparticles in a liquid. The Brownian motion of nanoparticles in suspension results in laser light to be scattered at different intensities. Analysing these intensity fluctuations allows to calculate the velocity of the Brownian motion. The size of the nanoparticles can be determined by using the Stokes-Einstein relationship. With the latest technology, it can measure nanoparticles smaller than 1 nm. The size of a nanoparticle may influence one or more functional properties of the nanoparticle including one or more of: its destination, biodistribution, tissue penetration, clearance, formation of a protein corona, toxicity, etc. Thus, the prediction of a nanoparticle size may advantageously provide information about the function of the nanoparticle in use, suitability of the nanoparticle for a particular use, etc.

Zeta potential is a measurement of the magnitude of the electrostatic or charge repulsion/attraction between particles. This can be determined by analysing particle mobility and charge (Zeta potential) using the Electrophoretic Light Scattering (ELS) technique. The zeta potential of a nanoparticle may influence one or more functional properties of the nanoparticle including one or more of: its destination, biodistribution, tissue penetration, clearance, formation of a protein corona, toxicity, etc. Thus, the prediction of a nanoparticle size may advantageously provide information about the function of the nanoparticle in use, suitability of the nanoparticle for a particular use, etc.

Any property as described herein may be normalised prior to use in training a model and/or prior to use as an input feature to a model for predictive purposes. Normalisation may be performed by dividing by a maximum value observed in a dataset. Normalisation may be performed by subtracting a minimum value observed in a dataset and dividing by a maximum value observed in a dataset (min-max normalisation).

### Functional Properties of Nanocarriers

The disclosure relates at least in part to the prediction of functional properties of nanocarriers. A functional property of a nanocarrier is a property that characterises the function of the nanocarrier independently of the activity or function of the payload (although in some cases the activity of the payload may be used to measure a functional property of the nanocarrier, such as e.g. when assessing transfection efficiency based on expression of a fluorescent marker encoded by a genetic payload).

The disclosure provides methods for predicting the transfection efficiency of a nanocarrier (also referred to herein as “transfection performance”). The transfection efficiency is a metric of the extent to which the nanocarrier is able to penetrate cells and deliver a payload. The exact value and units of a transfection efficiency is typically assay dependent. However, the relative transfection efficiencies of different nanocarriers in the same assay is representative of their relative transfection efficiencies across assays. For example, transfection efficiency may be measured using relative fluorescence units (RFU) observed after transfection of cells with nanocarriers comprising a genetic payload encoding a fluorescent protein. The RFU measured for a sample may be normalised, for example based on the number of cells or the measured amount of protein in the sample. This may be expressed for example as RFU/mg protein. Such a metric may be measured using any method known in the art, such as e.g. as described in Example 1 below. The RFU measurements (or normalised RFU) observed using the same protocol for different nanocarriers are indicative of the relative transfection efficiencies of these nanocarriers and can be used to train a model to predict transfection efficiency. While the actual value of the predicted transfection efficiency may not match the observed transfection efficiency in another assay, the predicted transfection efficiencies of a set of candidate nanocarriers can be used to compare these candidates in terms of their expected transfection efficiency, thereby enabling prioritisation of candidates for testing, further development, etc. The transfection efficiency may refer to in vivo transfection efficiency or in vitro transfection efficiency. The transfection efficiency may be a normalised transfection efficiency. A normalised transfection efficiency may be obtained by subtracting a control value (e.g. a value associated with a negative control, such as e.g. experiments using the same protocol but incubating the cells to be transfected with the nanocarrier vehicle without nanocarrier-such procedures may be referred to as background subtraction) and/or by dividing by a control (e.g. a value associated with a positive control such as e.g. experiments using a nanocarrier known to have stable transfection efficiency). The transfection efficiency for a nanocarrier and/or a control may be a summarised value across experiments such as e.g. a mean or median value. Experiments with transfection efficiency values below a predetermined cut-off, such as e.g. a number of standard deviations (e.g. 1 or 2 standard deviations) above a negative control (i.e. background) may be excluded from training data.

Instead or in addition to transfection efficiency, the methods described herein are applicable to predict any one or more of the following functional properties: cell-specific payload delivery (e.g. monocytes/macrophages), tissue specific payload delivery, in vitro and/or in vivo cytotoxicity (e.g. using live/dead staining data in vitro as training data), in vitro and/or in vivo immunogenicity, nanocarrier temperature dependent structural stability and integrity (e.g. using training data from freeze-thaw experiments to assess how stability is affected), nanocarrier pH dependent structural stability, integrity, performance, and behaviour (e.g. using training data from experiments in which the nanocarriers are incubated in low pH such as eg. pH≈5—this may be useful to predict the state of the nanoparticles in the lysosome compared to a more neutral pH environment such as blood/cytoplasm), and nanocarrier concentration dependent structural stability, integrity, performance, and behaviour (e.g. using training data comprising DLS and transfection performance at various concentrations). Cell-specific payload delivery may be predicted by training one or more machine learning models to predict transfection performance in one or more of a plurality of cell-types (e.g. using training data comprising transfection efficiency data for a plurality of nanocarriers in a plurality of cell types). A machine learning model may be trained to predict cell-specific payload delivery (e.g. transfection efficiency) in each of a plurality of cell types, using training data from said plurality of cell types. Alternatively, a machine learning model may be trained in a cell-type specific manner, to predict cell-specific payload delivery (e.g. transfection efficiency) in a specific cell type, using training data from said cell type. The predicted transfection performances may then be compared to assess whether transfection performance is significantly higher in a particular cell type or group of cell types. Alternatively, the predicted transfection performances in each of a plurality of cell types may be assessed to determine whether transfection efficiency is negligible in one or more cell types and not in one or more other cell types. Negligible transfection efficiency may be considered to be any predicted transfection efficiency equal to or below background fluorescence or below any predetermined performance threshold such as e.g. a performance threshold considered adequate for taking forward for in vivo studies or therapeutics. Instead or in addition to this, a machine learning model may have been trained to predict a categorical variable indicative of whether the predicted transfection performance in each of one or more cell lines is above or below a predetermined threshold (e.g. using training data in which transfection performance at or below the predetermined threshold, e.g. background fluorescence, are labelled with a first value and transfection performance above the predetermined threshold are labelled with a second value). Tissue specific payload delivery may be predicted in a similar manner using training data comprising data for tissue specific reporter expression in one or more tissues. The tissue specific reporter expression may be used as a marker of transfection efficiency, and a machine learning model may be trained to predict this (either as a continuous variable or as a categorical variable) for each of one or more tissues. Cytotoxicity may be predicted using a machine learning model trained to predict cytotoxicity using training data comprising data from a live/dead staining experiment (e.g. % live cells after exposure to the nanocarrier). Immunogenicity may be predicted using a machine learning model trained to predict immunogenicity using training data comprising data from any known immunogenicity assay (e.g. an assay quantifying T cell activation after exposure to the nanocarrier or measuring levels of inflammatory response signatures in serum after in vivo use of the nanocarrier). Nanocarrier temperature dependent structural stability and integrity may be predicted using a machine learning model trained to predict the size and/or PDI of the nanocarrier and/or change in size and/or change in PDI of the nanocarrier after exposure to low temperatures for one or more predetermined periods of time (e.g. change in PDI after 1 day, 1 week, 1 month at low temperature), or categorical variables indicating whether a change above or below a predetermined threshold was observed. Nanocarrier pH dependent structural stability and integrity may be predicted using a machine learning model trained to predict the size and/or PDI of the nanocarrier and/or change in size and/or change in PDI of the nanocarrier when in low pH solutions, or categorical variables indicating whether a change above or below a predetermined threshold was observed. This may be informative of lysosomal/endosomal escape of the nanocarrier/payload, especially for histidine containing designs which become charged at low pH. Nanocarrier concentration dependent structural stability, integrity, and performance may be predicted using a machine learning model trained to predict the size and/or PDI and/or transfection efficiency of the nanocarrier and/or change in size and/or change in PDI and/or change in transfection efficiency of the nanocarrier wat various concentrations (such as e.g. between a low concentration and a high concentration) solutions, or categorical variables indicating whether a change above or below a predetermined threshold was observed. A low concentration may be a concentration typically used for in vitro assays and a high concentration may be a concentration typically used for in vivo assays. Such a model may provide information about which nanocarriers may not be suitable for in vivo use (where higher concentrations are typically required compared to those used in vitro).

The terms “features”, “characteristics” and “properties” are used interchangeably herein.

### Prediction of Nanocarrier Properties

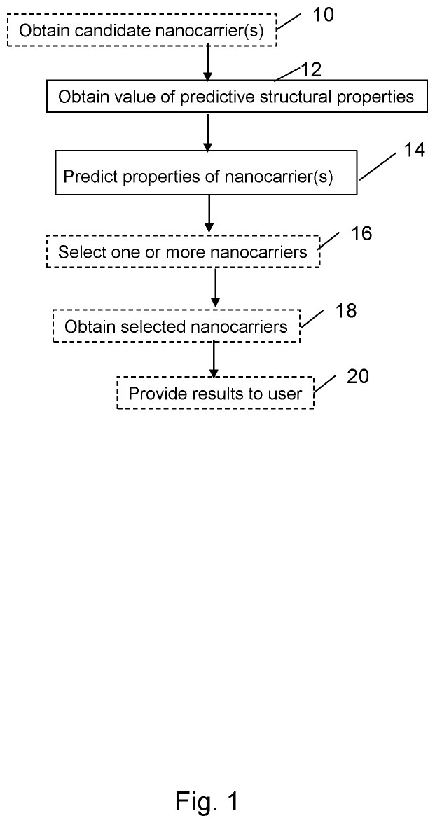

FIG. 1 shows a flow diagrams illustrating, in schematic form, a method for predicting one or more properties of a nanocarrier and/or for selecting or providing a nanocarrier according to the disclosure. At optional step 10, one or more candidate nanocarriers may be obtained, at a processor. Candidate nanocarriers may also be referred to as ‘candidate nanocarrier formulations’. Obtaining a candidate nanocarrier comprises receiving information about the composition of the candidate nanocarrier. For example, obtaining a candidate nanocarrier may comprise receiving information about the type of lipids, protein/peptide (if present) and cargo comprised in the nanocarrier, as well as their quantities or relative quantities. A candidate nanocarrier may be received from a user (e.g. through a user interface), retrieved from a memory or data store, or received from a computing device. The step of obtaining one or more candidate nanocarriers may comprise designing one or more candidate nanocarriers. Designing one or more candidate nanocarriers may be performed for example by a processor (which may be the same or a different processor from the one receiving the candidate nanocarrier(s)). The processor designing the one or more candidate nanocarriers may randomly select components and or relative quantities for a nanocarrier, using predetermined rules (such as e.g. sampling from predetermined relative amounts or ranges, predetermined types of lipids, predetermined peptide lengths, predetermined amino acid compositions for peptides, etc.). Alternatively, designing one or more candidate nanocarriers may be performed by an expert. Alternatively, the step of designing one or more candidate nanocarriers may be performed by a processor based on input from an expert. For example, the processor may receive an input comprising a selection of peptides and lipids, and the processor may select (e.g. randomly or exhaustively) a set of quantities of the components of the nanocarrier to use for each of a plurality of candidate nanocarriers. At step 12, the value of one or more structural properties and/or functional properties of the nanocarrier(s) is obtained by the processor. Obtaining the value of one or more properties of the nanocarrier(s) may comprise one or more of: the processor receiving a measured value of one or more properties of the nanocarrier(s), the processor determining the value of one or more properties of the nanocarrier(s) (e.g. by calculating the value of one or more properties and/or analysing the composition of the one or more nanocarriers to determine the value of one or more properties), the processor receiving the value of one or more properties of the nanocarrier(s) (such as from a user through a user interface, from a memory or data store, or from another computing device), and the processor predicting the value of one or more properties of the nanocarrier(s) (e.g. using one or more machine learning models trained to predict the value of said properties, or using one or more machine learning models trained to predict the value of learned features from e.g. 2D or 3D structural information about one or more components of the nanocarrier). Step 12 may further comprise the step of measuring the value of one or more structural and/or functional properties of the nanocarrier(s). The values of the structural properties obtained at step 12 may be referred to as “input structural properties’. The values of the functional properties obtained at step 12 may be referred to as “input functional properties’. Step 12 may further comprise a step of pre-processing the determined, measured and/or predicted values of structural/functional properties. For example, the values may be normalised, for example by dividing by a maximum possible or observed value and/or by subtracting a determined value by a minimum possible or observed value. Observed values may refer to values observed in a training data set. Possible values may refer to values that are theoretically possible. Step 12 may further comprise summarising a plurality of values (e.g. replicate measurements) and/or filtering values or nanocarrier(s) to exclude nanocarriers that do not satisfy one or more predetermined criteria (e.g. measured values below a predetermined threshold, e.g. indicative of background signal).

At step 14, the values obtained at step 12 are used to predict one or more properties of the nanocarrier(s). The one or more properties include at least one functional property, preferably at least the transfection efficiency. These may be referred to as ‘output functional properties’ by contrast with the input functional properties obtained at step 12. The one or more properties may include one or more structural properties, such as e.g. the size and/or PDI of the nanocarrier. These may be referred to as ‘output structural properties’ by contrast with the input structural properties obtained at step 12. At optional step 16, one or more nanocarriers of the set of candidate nanocarriers may be selected, using the output of step 14. For example, the candidate nanocarriers may be ranked based on transfection efficiency (or any other functional properties), and a predetermined number of highest ranked nanocarriers, highest ranked nanocarriers that satisfy one or more further criteria (e.g. criteria applying to one or more predicted properties other than the transfection efficiency), or predetermined number of nanocarriers amongst a predetermined number of highest ranked nanocarriers (e.g. 3 nanocarriers amongst the top 10 ranked nanocarriers) may be selected. The selected nanocarriers may be further evaluated in silico, in vitro and/or in vivo. For example, the selected nanocarriers may be formulated (i.e. physically obtained), and one or more structural and/or functional properties of the selected nanocarriers may be tested. A further subset of the selected nanocarriers may then be selected for further testing and/or optimisation. At optional step 20, the results of any of steps 12, 14, 16 and 18 may be provided to a user.

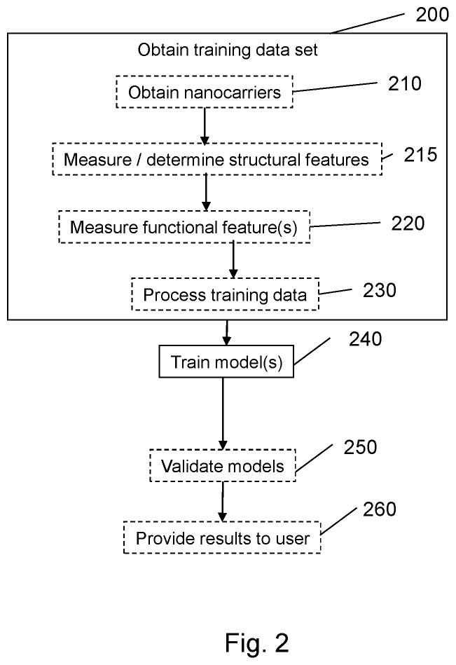

FIG. 2 shows a flow diagrams illustrating, in schematic form, a method for providing a tool for predicting one or more properties of a nanocarrier and/or for selecting a nanocarrier according to the disclosure. At step 200, a training data set is obtained by a processor comprising, for each of a plurality of nanocarriers comprising, for each of a plurality of nanocarriers (such as e.g. at least 100 different nanocarrier formulations, preferably at least 150, optionally comprising at least 20, at least 30 or at least 40 different compositions in terms of the identity of the components of the nanocarriers): experimental data quantifying one or more functional properties of the nanocarrier; experimental and/or in silico determined values of one or more structural properties of the nanocarrier. The training data may be received by the processor from a user (e.g. through a user interface), from a memory or database, or from one or more computing devices. The method may further comprise the steps of determining the values of the features, through optional step 210 of obtaining the nanocarriers (e.g. by formulating the nanocarriers), optional step 215 or measuring or determining the value of the structural properties and optional step 220 of measuring the functional properties. Typically, determined structural properties are advantageously used as input structural features of a trained machine learning model, and measured functional and structural features are advantageously used as output features of a trained machine learning model. Thus, determined structural properties may be used as predictive variables to predict the value of one or more structural and/or functional features that are measured (ground truth values). At optional step 230, the values obtained at steps 215 and 220 may be pre-processed, such as e.g. by excluding values that do not satisfy predetermined criteria (e.g. failed transfections, values below a predetermined background level) and/or summarising values (e.g. across replicates) and/or normalising values. Excluded values may be removed from the training data set (e.g. excluding a complete data series for a nanocarrier) or may be replaced by suitable default values (e.g. any failed transfection or value below background may be replaced by a “0” or any predetermined value representative of background level). At step 240, one or more machine learning models may be trained to predict the values of one or more functional properties and optionally one or more experimentally determined structural properties of nanocarriers including in the training data set using inputs comprising the values of one or more properties (structural and/or functional properties, preferably including or consisting of structural properties) of the nanocarriers (whether determined or measured) including in the training data set. The one or more machine learning models may comprise an ensemble of models trained to predict the same properties, the output of which are combined to obtain the predictions. The one or more machine learning models may comprise a plurality of models trained to predict different sets of one or more properties. The one or more machine learning models may comprise machine learning models with different architectures (e.g. different types of models such as multiple linear regression models, random forest models, artificial neural networks, ANNs with different number of layers and/or neurons, etc.). The one or more machine learning models may comprise machine learning models using different sets of input features. When multiple models are trained to predict the same features, these may be compared in terms of e.g. prediction performance on the training data set, This may be performed using methods known in the art, such as e.g. by evaluation prediction loss by cross-validation. At optional step 250, the one or more models trained at step 240 (or a selected subset thereof) may be validated or further validated (such as e.g. using leave one out cross-validation, using a validation data set that was not used for training of the models, etc). At step 260, the results of any of steps 200, 240 and 250 may be provided to a user, e.g. through a user interface. This may include one or more of: information about the training data (i.e. any information obtained at step 200 or any information derived therefrom such as number of nanocarriers and summary properties of the set of nanocarriers, e.g. distributions or statistics of one or more of the features obtained for the nanocarriers), information about the models (e.g. any information obtained at step 240 or derived therefrom such as prediction performance, training parameters, model parameters such as architecture, weights, inputs, outputs, etc.) and information about the validation (e.g. validation performance, validation data used, etc.). This may in particular comprise the parameters of one or more trained models, which may be used by a user to implement the methods described herein such as e.g. by reference to FIG. 1.

As used herein, the term “machine learning model” refers to a mathematical model that has been trained to predict one or more output values based on input data, where training refers to the process of learning, using training data, the parameters of the mathematical model that result in a model that can predict outputs values that satisfy an optimality criterion or criteria, such as e.g. providing predictions with minimal error compared to comparative (known) values associated with the training data (where these comparative values are commonly referred to as “labels” or “ground truth”). The term “machine learning algorithm” or “machine learning method” refers to an algorithm or method that trains and/or deploys a machine learning model. “Classifier” or “classification algorithm” may be a machine learning model or algorithm that maps input data, such as one or more structural features, to a category, such as immunogenic/non immunogenic, transfection efficiency above/below a threshold, etc. A classifier may produce as output a probabilistic score, which reflects the likelihood that an observation belongs to particular category. The machine learning approaches used herein may be termed “supervised” as a training set of samples with known class or outcome is used to produce a mathematical model which is then evaluated with independent validation data sets. Here, a “training set” of nanocarrier measurements, e.g. measured or calculated nanocarrier structural and functional features, is used to construct a statistical model that predicts correctly the class of each sample. This training set is then tested with independent data (referred to as a test or validation set) to determine the robustness of the computer-based model. The robustness of the predictive models can also be checked using cross-validation, by leaving out selected samples from the analysis.

A model that predicts one or more properties of a nanocarrier may be trained by using a learning algorithm to identify a function F:v, p=>kᵢ where F is a function parameterised by a set of parameters θ such that: kᵢ≈\hat{k}ᵢ=F(v|θ) where \hat{ƒ}ᵢ is a predicted property, v is a set of predictive features (structural properties of the nanocarrier), and θ is a set of parameters identified as satisfying: θ=argmin_{θ}L(kᵢ, \hat{k}ᵢ) where L is a loss function that quantifies the model prediction error based on the observed and predicted nanocarrier properties. The specific choice of the function F, parameters θ and function L as well as the specific algorithm used to find θ (learning algorithm) depends on the specific machine learning method used. Any method that satisfies the equations above can be used within the context of the present disclosure, including in particular any choice of loss function, model type and architecture. In embodiments, a statistical model that may be used to predict one or more properties of a nanocarrier is a linear regression model. A linear regression model is a model of the form according to the following equation: Y=Xβ+ε which can also be written as yᵢ=β₀+β₁xᵢ₁+ . . . . βₚxᵢₚ+εᵢ i=1, . . . , n where Y is a vector with n elements yi (one for each dependent/predicted variable), X is a matrix with elements xᵢ₁ . . . . xᵢₚ for each of the p predictor variables and each of the n dependent variables, and n elements of 1 for the intercept value, β is a vector of p+1 parameters, and & is a vector of n error terms (one for each of the dependent variables).

In embodiments, a machine learning model is a non-linear model such as a non-linear regressor or classifier. In embodiments, a machine learning model is a random forest regressor. A random forest regressor is a model that comprises an ensemble of decision trees and outputs a class that is the average prediction of the individual trees. Decision trees perform recursive partitioning of a feature space until each leaf (final partition sets) is associated with a single value of the target. Regression trees have leaves (predicted outcomes) that can be considered to form a set of continuous numbers. Random forest regressors are typically parameterized by finding an ensemble of shallow decision trees. For example, random forests can be used to predict the value of one or more properties of a nanocarrier. In embodiments, a machine learning model is an artificial neural network (ANN, also referred to simply as “neural network” (NN)). ANNs are typically parameterized by a set of weights that are applied to the inputs of each of a plurality of connected neurons in order to obtain a weighted sum that is fed to an activation function to produce the neuron's output. The parameters of an NN can be trained using a method called backpropagation through which connection weights are adjusted to compensate for errors found in the learning process, in combination with a weight updating procedure such as stochastic gradient descent. The ANN may be a deep neural network. A deep neural network is a network with more than one hidden layers. The ANN may be a convolutional neural network (CNN).

Suitable loss functions for the training of machine learning models such as those described herein include the mean squared error, the mean absolute error and the Huber loss. The mean squared error (MSE) can be expressed as: L(·)=MSE(kᵢ, \hat{k}ᵢ)=(kᵢ−\hat{k}ᵢ)². The mean absolute error (MAE) can be expressed as:

L(·)=MAE(kᵢ,\hat{k}ᵢ)=|kᵢ−\hat{k}ᵢ|

The MAE is believed to be more robust to outlier observations than the MSE. The MAE may also be referred to as “L1 loss function”. The Huber loss can be expressed as:

$$L ( {k}_{i} , {\hat{k}}_{i} \vert a ) = \{ \begin{matrix}0.5 {( k - {k}_{i} )}^{2} & for \vert {k}_{i} - {\hat{k}}_{i} \vert < a \\ a \vert {k}_{i} - {k}_{i} \vert - 0.5 {a}^{2} & otherwise\end{matrix}$$

where α is a parameter. The Huber loss is believed to be more robust to outliers than MSE, and strongly convex in the neighborhood of its minimum. However, MSE remains a very commonly used loss function especially when a strong effect from outliers is not expected, as it can make optimization problems simpler to solve. In embodiments, the loss function used is an L1 loss function. In embodiments, the loss function used is a smooth loss function. Smooth loss functions are convex in the vicinity of an optimum, thereby making training easier.

The machine learning model may comprise a plurality of individual machine learning models wherein each individual machine learning model has been trained to take as input one or more structural properties of a nanocarrier and produce as output a different one or more functional and/or structural properties for the nanocarrier. Alternatively, the machine learning model may comprise a single model that has been trained to take as input one or more structural properties of a nanocarrier and produce as output all of or a plurality of functional and/or structural properties to be predicted for the nanocarrier. In other words, separate models may be trained to predict each of a plurality of properties of interest, or a single model may be trained to jointly predict a plurality of properties of interest. In such cases, the loss function used may be modified to be an (optionally weighted) average across all variables that are predicted, as described in the following equation:

$${L}_{M} ( k , \hat{k} ) = \frac{1}{n} {\sum }_{i ∈ m} {α}_{i} L ( {k}_{i} , {\hat{k}}_{i} )$$

where αᵢ are optional weights that may be individually selected for each of the features i, k and \hat{k} are the vectors of actual and predicted features. Optionally, the values of k; may be scaled prior to inclusion in the loss function (e.g. by normalising so that the labels for all the jointly predicted variables have equal variance), for example to reduce the risk of some of the jointly predicted \hat{k}ᵢ dominating the training.

A machine learning model as described herein may comprise an ensemble of models whose predictions are combined. Alternatively, a machine learning model may comprise a single model. The machine learning model may comprise one or more ensembles of individual machine learning models. The outputs produced for the same nanocarrier as output by each individual machine learning model in an ensemble may be combined into a single prediction for each ensemble, for example a mean or median prediction. All the individual machine learning models may have the same architecture. Each individual machine learning model may have been independently trained. In other words, the parameters of the individual machine learning models may differ (due to training), even where the general architecture of the individual machine learning models is the same. Each individual machine learning model may have been trained using training data comprising structural features for a plurality of nanocarriers and corresponding measurements of the structural and/or functional features of the nanocarrier to be predicted.

A machine learning model as described herein may use as input(s) (also referred to as input features/values/variables, predictive features/values/variables or independent features/values/variables) any one or more of the features listed in Table 1. For example, a machine learning model as described herein may take as input(s) any one or more of: the protein to payload ratio (e.g. N/P ratio-ratio of positively charged amine nitrogen (N) in a peptide component of the nanocarrier to phosphate in a nucleic acid payload), the number of positively charged sidechains in a peptide component of the nanocarrier, the number of negatively charged sidechains in a peptide component of the nanocarrier, the number of generations in a branched dendrimer component of the nanocarrier, the molecular weight of any component of the nanocarrier (such as e.g. the molecular weight of a peptide component of the nanocarrier), the number of a particular amino acid in a peptide component of the nanocarrier (e.g. number of His, number of Cys), a score indicative of the hydrophilicity of residues in a peptide component (such as e.g. all residues or only hydrophobic residues) and/or summary values derived therefrom (e.g. sum, average), the percentage or proportion of hydrophobic residues in a peptide component, the percentage or proportion of hydrophilic residues in a peptide component, the presence of a particular type of residues in a particular region of a peptide component (e.g. presence of cysteine residues and/or positively charge residues in a dendrimer core sequence). Each of the input features may be normalised, e.g. by dividing by a maximum possible or observed value and/or by subtracting by a minimum possible or observed value. Thus, the input values may be unitless. The input features may each individually be categorical variables, Boolean variables, discrete variables or continuous variables. Further, the same variable may be input as a continuous variable or as a categorical or Boolean variable (e.g. by comparison with a one or more predetermined thresholds to assign categories to continuous values). Unless indicated otherwise, values such as ratios, proportion, percentages, molecular weight, and hydrophilicity/hydrophobicity are continuous variables. Unless indicated otherwise, values such as numbers of residues, numbers of generations and any other counts/numbers are discrete variables. Unless indicated otherwise, values such as presence/absence of a particular feature are Boolean variables.

In embodiments, the input variables include at least one of: the protein/peptide to payload ratio (e.g. N/P ratio), the number of positively charged sidechains in a peptide component of the nanocarrier, the molecular weight of a peptide component, and a value indicative of the hydrophilicity of the peptide component (e.g. sum of hydrophilicity scores from each residue in the peptide component). Preferably, the input variables include at least the protein to payload ratio (e.g. N/P ratio).

A machine learning model as described herein may produce as output(s) (also referred to as output features/values/variables, predicted features/values/variables or dependent features/values/variables) any one or more of the functional features of nanocarriers described herein. A machine learning model as described herein may produce as output(s) any one or more of the structural nanocarrier properties described herein. For example, a machine learning model as described herein may produce as output(s) one or more of: transfection efficiency (or normalised transfection efficiency, e.g. unitless), size (e.g. hydrodynamic size, e.g. in nm), and PDI (unitless). Advantageously, a machine learning model as described herein may produce as output(s) any one or more of the structural nanocarrier properties described herein that are measured properties of the nanocarrier. For example, properties such as size, PDI or zeta potential cannot be exactly calculated theoretically and hence are beneficially predicted. Indeed, prediction of such features removes the need to synthesise the nanocarrier and measure said features. Conversely, a machine learning model as described herein may not produce as output(s) any one or more of the structural nanocarrier properties described herein that can be theoretically calculated. For example, features such as sequence length or number of residues or a particular type can be calculated theoretically for any candidate nanocarrier. Therefore, prediction of such features does not serve much purpose.

“Computer-implemented method” where used herein is to be taken as meaning a method whose implementation involves the use of a computer, computer network or other programmable apparatus, wherein one or more features of the method are realised wholly or partly by means of a computer program. The systems and methods described herein may be implemented in a computer system, in addition to the structural components and user interactions described. As used herein, the term “computer system” includes the hardware, software and data storage devices for embodying a system or carrying out a method according to the above-described embodiments. For example, a computer system may comprise a processing unit, such as a central processing unit (CPU) and/or a graphics processing unit (GPU), input means, output means and data storage, which may be embodied as one or more connected computing devices. Preferably the computer system has a display or comprises a computing device that has a display to provide a visual output display. The data storage may comprise RAM, disk drives or other computer readable media. The computer system may include a plurality of computing devices connected by a network and able to communicate with each other over that network. It is explicitly envisaged that computer system may consist of or comprise a cloud computer. The methods described herein may be provided as computer programs or as computer program products or computer readable media carrying a computer program which is arranged, when run on a computer, to perform the method(s) described herein. As used herein, the term “computer readable media” includes, without limitation, any non-transitory medium or media which can be read and accessed directly by a computer or computer system. The media can include, but are not limited to, magnetic storage media such as floppy discs, hard disc storage media and magnetic tape; optical storage media such as optical discs or CD-ROMs; electrical storage media such as memory, including RAM, ROM and flash memory; and hybrids and combinations of the above such as magnetic/optical storage media.

### Systems

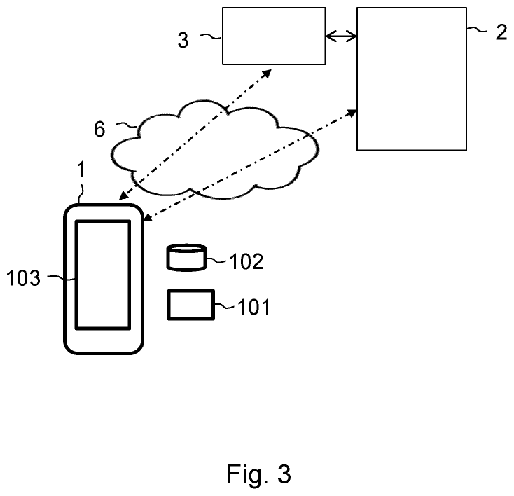

FIG. 3 shows an embodiment of a system for predicting one or more properties of a nanocarrier and/or selecting, providing or designing a nanocarrier according to the disclosure. The system comprises a computing device 1, which comprises a processor 101 and computer readable memory 102. In the embodiment shown, the computing device 1 also comprises a user interface 103, which is illustrated as a screen but may include any other means of conveying information to a user such as e.g. through audible or visual signals. The computing device 1 may be communicably connected, such as e.g. through a network, to nanocarrier data acquisition means 3, such as e.g. a dynamic light scattering device, a fluorescence microscope, etc., and/or to one or more databases 2 storing nanocarrier data. The one or more databases 2 may further store one or more of: training data, parameters (such as e.g. parameters of a machine learning model used to predict nanocarrier properties, e.g. weights of neural network, architecture and parameters of a decision tree or neural network model, etc.), experimental and/or sample related information, control sample information, etc. The computing device may be a smartphone, tablet, personal computer or other computing device. The computing device is configured to implement a method for predicting one or more properties of a nanocarrier, as described herein. In alternative embodiments, the computing device 1 is configured to communicate with a remote computing device (not shown), which is itself configured to implement a method of predicting one or more properties of a nanocarrier, as described herein. In such cases, the remote computing device may also be configured to send the result of the method to the computing device. Communication between the computing device 1 and the remote computing device may be through a wired or wireless connection, and may occur over a local or public network 6 such as e.g. over the public internet. The data acquisition means may be in wired connection with the computing device 1, or may be able to communicate through a wireless connection, such as e.g. through WiFi and/or over the public internet, as illustrated. The connection between the computing device 1 and the data acquisition means 3 may be direct or indirect (such as e.g. through a remote computer). The data acquisition means 3 are configured to acquire structural and/or functional data from nanocarrier samples, using in vitro or in vivo assays.

### Uses of Nanocarriers

Nanoparticles as described herein may be used in a variety of contexts, to deliver a variety of payloads.

Messenger RNA (mRNA)

Messenger RNA (mRNA) is a single-stranded molecule of RNA that takes the coding sequence of a gene to be translated into the corresponding amino acid sequence by a ribosome. mRNA is created during the process of transcription, where an enzyme (RNA polymerase) converts the gene into primary transcript mRNA (also known as pre-mRNA). This pre-mRNA usually still contains introns, regions that will not go on to code for the final amino acid sequence. These are removed in the process of RNA splicing, leaving only exons, regions that will encode the protein. This exon sequence constitutes mature mRNA. Mature mRNA is then read by the ribosome, thereby producing the encoded protein. The invention can be used to deliver mRNA molecules to target cells and tissues as a means of inducing expression of a desired protein or peptide. Inducing peptide/protein expression via mRNA delivery is particularly useful when transient expression is desired. To improve mRNA expression, synonymous codons within mRNA may be changed based on an organism's codon bias—i.e. the mRNA may be codon optimized. For example, the mRNA may be codon optimized for expression in the organism type of the subject to be administered the composition of the invention. For example, the mRNA may be codon optimised for expression in a mammal, for example a human.

In particular, delivery of mRNA encoding chimeric antigen receptors (CARs) and transcription factors are envisaged.

mRNAs and lncRNAs (discussed below) are typically large molecules with a negatively charged side and a hydrophobic side. mRNAs and lncRNAs will therefore require a balance between hydrophobic and hydrophilic interactions to be encapsulated and delivered to target tissues and cells. This balance between hydrophobic and hydrophilic interactions will be different from, for example, double stranded nucleic acid such as pDNA and siRNA which has charge on both sides. As mRNAs and lncRNAs are significantly larger than, for example, ASOs the requirement for encapsulation and delivery will also likely be different. As such the optimal NP ratio of dendrimer and w/w ratio of DOTMA/DOPE for mRNA and lncRNA delivery will differ compared to ASO delivery.

### RNA Interference

Nanoparticles of the disclosure may facilitate the therapeutic down regulation of target gene expression via delivery of nucleic acids. These include RNA interference (RNAi). Small RNA molecules may be employed to regulate gene expression.

These include targeted degradation of mRNAs by small interfering RNAs (siRNAs), post transcriptional gene silencing (PTGs), developmentally regulated sequence-specific translational repression of mRNA by micro-RNAs (miRNAs) and targeted transcriptional gene silencing.

A role for the RNAi machinery and small RNAs in targeting of heterochromatin complexes and epigenetic gene silencing at specific chromosomal loci has also been demonstrated. Double-stranded RNA (dsRNA)-dependent post transcriptional silencing, also known as RNA interference (RNAi), is a phenomenon in which dsRNA complexes can target specific genes of homology for silencing in a short period of time. It acts as a signal to promote degradation of mRNA with sequence identity. A 21-nt siRNA is generally long enough to induce gene-specific silencing, but short enough to evade host response. The decrease in expression of targeted gene products can be extensive with 90% silencing induced by a few molecules of siRNA.

In the art, these RNA sequences are termed “short or small interfering RNAs” (siRNAs) or “microRNAs” (miRNAs) depending on their origin. Both types of sequence may be used to down-regulate gene expression by binding to complementary RNAs and either triggering mRNA elimination (RNAi) or arresting mRNA translation into protein. siRNA are derived by processing of long double stranded RNAs and when found in nature are typically of exogenous origin. Micro-interfering RNAs (miRNA) are endogenously encoded small non-coding RNAs, derived by processing of short hairpins. Both siRNA and miRNA can inhibit the translation of mRNAs bearing partially complimentary target sequences without RNA cleavage and degrade mRNAs bearing fully complementary sequences.

Accordingly, these sequences may be used in a composition as described herein for down-regulating the expression of a target gene. For example, it is envisaged that the nanoparticles designed as described herein can be used to deliver RNAi-based therapy for use in treating diabetes, for example type I or type II diabetes.

The siRNA ligands are typically double stranded and, in order to optimise the effectiveness of RNA mediated down-regulation of the function of a target gene, it is preferred that the length of the siRNA molecule is chosen to ensure correct recognition of the siRNA by the RISC complex that mediates the recognition by the siRNA of the mRNA target and so that the siRNA is short enough to reduce a host response.

miRNA ligands are typically single stranded and have regions that are partially complementary enabling the ligands to form a hairpin. miRNAs are RNA genes which are transcribed from DNA, but are not translated into protein. A DNA sequence that codes for a miRNA gene is longer than the miRNA. This DNA sequence includes the miRNA sequence and an approximate reverse complement. When this DNA sequence is transcribed into a single-stranded RNA molecule, the miRNA sequence and its reverse-complement base pair to form a partially double stranded RNA segment. The design of microRNA sequences is discussed on John et al, 2004.

Typically, the RNA ligands intended to mimic the effects of siRNA or miRNA have between 10 and 40 ribonucleotides (or synthetic analogues thereof), more preferably between 17 and 30 ribonucleotides, more preferably between 19 and 25 ribonucleotides and most preferably between 21 and 23 ribonucleotides. In some embodiments of the invention employing double-stranded siRNA, the molecule may have symmetric 3′ overhangs, e.g. of one or two (ribo) nucleotides, typically a UU of dTdT 3′ overhang. Based on the disclosure provided herein, the skilled person can readily design suitable siRNA and miRNA sequences, for example using resources such as Ambion's online siRNA finder. siRNA and miRNA sequences can be synthetically produced and added exogenously to cause gene downregulation or produced using expression systems (e.g. vectors). In a preferred embodiment the siRNA is synthesized synthetically.

Longer double stranded RNAs may be processed in the cell to produce siRNAs (see for example Myers et al (2003)). The longer dsRNA molecule may have symmetric 3′ or 5′ overhangs, e.g. of one or two (ribo) nucleotides, or may have blunt ends. The longer dsRNA molecules may be 25 nucleotides or longer. Preferably, the longer dsRNA molecules are between 25 and 30 nucleotides long. More preferably, the longer dsRNA molecules are between 25 and 27 nucleotides long. Most preferably, the longer dsRNA molecules are 27 nucleotides in length.

In one embodiment, the siRNA, longer dsRNA or miRNA is produced endogenously (within a cell) by transcription from a vector. The vector may be introduced into the cell in any of the ways known in the art. Optionally, expression of the RNA sequence can be regulated using a tissue specific promoter. In a further embodiment, the siRNA, longer dsRNA or miRNA is produced exogenously (in vitro) by transcription from a vector.

Alternatively, siRNA molecules may be synthesized using standard solid or solution phase synthesis techniques which are known in the art. Linkages between nucleotides may be phosphodiester bonds or alternatives, for example, linking groups of the formula P(O)S, (thioate); P(S)S, (dithioate); P(O)NR′2; P(O)R′; P(O)OR6; CO; or CONR′2 wherein R is H (or a salt) or alkyl (1-12C) and R6 is alkyl (1-9C) is joined to adjacent nucleotides through —O— or —S—.

### Long Non-Coding RNA

Mammalian genomes are pervasively transcribed, producing a vast array of transcripts including many thousands of long non-coding RNA molecules (lncRNAs). It has been shown that lncRNAs can regulate the chromatin state, transcription, RNA stability, and the translation of certain genes.

### RNA Activation (RNAa)

RNA activation (RNAa) is a process mediated by RNAs to enhance gene expression via a highly regulated and evolutionarily conserved pathway. RNAa can be induced by small activating RNA (saRNA), which is a class of noncoding RNA consisting of a 21-nucleotide dsRNA with 2-nucleotide overhangs at both ends. saRNA has an identical structure and chemical components to siRNA despite the fact that saRNA mediates gene activation in a sequence specific manner. To activate gene expression, the guide strand of the saRNA is loaded to AGO2, and the complex is then transported to the nucleus. Once in the nucleus, the guide strand-AGO2 complex binds directly to gene promoters or associated transcripts, recruiting key components including RNA polymerase II to initiate gene activation (Kwok et al. 2019).

### Antisense Oligonucleotides (ASOs)

Antisense oligonucleotides (ASOs) are single strands of DNA or RNA that are complementary to a target sequence. The ASO hybridises with the target nucleic acid. For instance, an ASO can be used to target a coding or non-coding RNA molecule in the cell. Following target binding, the ASO/target complex may be enzymatically degraded, e.g. by RNase H.

### Circular RNA

The nanoparticles of the disclosure may use circular RNA (circRNA) as a nucleic acid component/payload. circRNA is a type of single-stranded RNA which forms a continuous closed loop due to a covalent bond being formed between the 5′ and 3′ ends of the RNA molecule. The closed loop structure of circRNA, and the lack of a Poly-A tail, are predicted to confer exonuclease resistance and thereby increases circRNA stability. Consequently, circRNA have an increased half-life compared to comparable non-circular RNA. For example, circRNAs which arise from a protein coding gene as an alternative splice form are more stable than the corresponding linear mRNA of the same protein coding gene. A number of functions have been ascribed to circRNAs including protein complex scaffolding, parental gene modulation, RNA-protein interactions, and microRNA sponges. Recently, it has been realised that circRNA may be useful in a range of therapeutic approaches. For example, circRNAs may be used as microRNA “sponges” to sequester microRNAs. circRNAs may also be used as sources of protein translation which can persist in cells longer than standard linear mRNAs. circRNAs may also be used to control protein activity by acting as aptamers.

### Modified Nucleic Acids

Modified nucleotide bases can be used in addition to the naturally occurring bases, and may confer advantageous properties on nucleic acids containing them.

For example, modified bases may increase the stability of the nucleic acid molecule, thereby reducing the amount required. The provision of modified bases may also provide nucleic acid molecules which are more, or less, stable than unmodified nucleic acids.

The term ‘modified nucleotide base’ encompasses nucleotides with a covalently modified base and/or sugar. For example, modified nucleotides include nucleotides having sugars which are covalently attached to low molecular weight organic groups other than a hydroxyl group at the 3′ position and other than a phosphate group at the 5′ position. Thus modified nucleotides may also include 2′ substituted sugars such as 2′-O-methyl-; 2′-O-alkyl; 2′-O-allyl; 2′-S-alkyl; 2′-S-allyl; 2′-fluoro-; 2′-halo or azido-ribose, carbocyclic sugar analogues, a-anomeric sugars; epimeric sugars such as arabinose, xyloses or lyxoses, pyranose sugars, furanose sugars, and sedoheptulose.

Modified nucleotides are known in the art and include alkylated purines and pyrimidines, acylated purines and pyrimidines, and other heterocycles. These classes of pyrimidines and purines are known in the art and include pseudoisocytosine, N4,N4-ethanocytosine, 8-hydroxy-N6-methyladenine, 4-acetylcytosine,5-(carboxyhydroxylmethyl) uracil, 5 fluorouracil, 5-bromouracil, 5-carboxymethylaminomethyl-2-thiouracil, 5-carboxymethylaminomethyl uracil, dihydrouracil, inosine, N6-isopentyl-adenine, 1-methyladenine, 1-methylpseudouracil, 1-methylguanine, 2,2-dimethylguanine, 2methyladenine, 2-methylguanine, 3-methylcytosine, 5-methylcytosine, N6-methyladenine, 7-methylguanine, 5-methylaminomethyl uracil, 5-methoxy amino methyl-2-thiouracil,-D-mannosylqueosine, 5-methoxycarbonylmethyluracil, 5-methoxyuracil, 2 methylthio-N6-isopentenyladenine, uracil-5-oxyacetic acid methyl ester, psueouracil, 2-thiocytosine, 5-methyl-2 thiouracil, 2-thiouracil, 4-thiouracil, 5-methyluracil, N-uracil-5-oxyacetic acid methylester, uracil 5-oxyacetic acid, queosine, 2-thiocytosine, 5-propyluracil, 5-propylcytosine, 5-ethyluracil, 5-ethylcytosine, 5-butyluracil, 5-pentyluracil, 5-pentylcytosine, and 2,6, diaminopurine, methylpsuedouracil, 1-methylguanine, 1-methylcytosine.

### Medical Therapies

The nanocarriers characterised according to the present disclosure may be used in gene therapy regimens. The use of gene therapy regimens employing either DNA or RNA, and in particular mRNA, are contemplated. The nucleic acid can be present in a composition which, when introduced into target cells, results in expression of a therapeutic gene product, e.g. a transgene. Target cells include myocytes, hepatocytes, stellate cells, brain cells (neurons, astrocytes), splenocytes, lung cells, cardiomyocytes, kidney cells, adipose cells, stem cells, monocytes, macrophages, dendritic cells, neutrophils, B cell, T cell, myeloid derived suppressor cells, tumour associated macrophages, tumour associated neutrophils or tumour cells.

For gene therapy to be practical, it is desirable to employ a DNA/RNA transfer system that: (1) directs the therapeutic sequence into the target cell, (2) mediates uptake of the therapeutic nucleic acid into a proportion of the target cell population, and (3) is suited for use in vivo and/or ex vivo for therapeutic application.

Nucleic acids encoding a transgene can express the transgene in a target cell. The transgene may be a protein or peptide. Additionally or alternatively, the nucleic acid can modulate expression or activity of an endogenous gene. The modulation can be an increase in the expression of the gene and/or exogenous expression of further copies of the gene, or the modulation can be a decrease in the expression of the gene.

The transgene may be a viral protein, a bacterial protein or a protein of a microorganism that is parasitic to a mammal. The composition expressing a viral protein, a bacterial protein or a parasitic microbial protein may be used as a vaccine. For instance, an effective amount of the composition may be delivered systemically to a subject (e.g. intravenously) to achieve expression of the viral protein, bacterial protein or parasitic microbial protein in the skeletal muscle of the subject in order to prime an immune response to that viral, bacterial or parasitic protein. Thus, this disclosure describes methods of vaccinating a subject, and methods for providing compositions for use in the vaccination of a subject, comprising characterising a nanocarrier using a method as described herein. In such examples, the transgene may be expressed in an immune cell described herein, e.g. a leucocyte, such as a B lymphocyte, a T lymphocyte, a monocyte, a neutrophil, a dendritic cell, a macrophage, or a monocyte; a lymph node tissue cell.

The transgene may be an immune molecule, for example, a T cell receptor, chimeric antigen receptor, a cytokine, a decoy receptor, an antibody, a costimulatory receptor, a costimulatory ligand, a checkpoint inhibitor, an immunoconjugate, or a tumour antigen.

The transgene may express a therapeutic protein for use in a gene therapy. The gene therapy may be for treating an autoimmune disorder, such as type I diabetes (also known as juvenile diabetes), cancers and/or a genetic disorder in a patient. The genetic disorder may be monogenic disorder, e.g. muscular dystrophy in the patient. The transgenes may be a function version of a gene that is non-functional, downregulated, inactive or impaired in a subject.

In embodiments where the monogenic disorder is a muscular dystrophy, the transgene may be dystrophin. In embodiments where the disorder is ischemia, the transgene may be hepatocyte growth factor (HGF), vascular endothelial growth factor (VEGF) and Fibroblast growth factors (FGF). In embodiments where the disorder is muscle wasting, the transgene may be follistatin. In embodiments where the disorder is a neuromuscular disease, the transgene may be acid α-glucosidase (GAA). While the transgene may be expressed in one or more of the tissues disclosed herein, the expressed protein may be secreted from the tissue(s) into the circulation.

It is envisaged that the nanocarriers characterised according to the invention can be used to deliver nucleic acid therapies to treat myopathies, It is also envisaged that this invention can be used to deliver nucleic acid therapies to treat muscular dystrophies such as Duchenne muscular dystrophy, myotonic dystrophy, facioscapulohumeral muscular dystrophy, Becker muscular dystrophy, limb-girdle muscular dystrophy, oculopharyngeal muscular dystrophy, Emery-Dreifuss muscular dystrophy, inheriting muscular dystrophy, congenital muscular dystrophy, and distal muscular dystrophy.

The nucleic acid therapy may be for treating muscle wasting conditions such as cachexia. The nucleic acid therapy may be for treating other muscular disorders, such as inherited muscular disorders, e.g. myotonia congenita, or familial periodic paralysis. The nucleic acid therapy may be for treating a motor neuron disease, such as ALS (amyotrophic lateral sclerosis), spinal-bulbar muscular atrophy (SBMA) or spinal muscular atrophy (SMA). The nucleic acid therapy may be for treating a mitochondrial disease, such as Friedreich's ataxia (FA), or a mitochondrial myopathy such as Kearns-Sayre syndrome (KSS), Leigh syndrome (subacute necrotizing encephalomyopathy), mitochondrial DNA depletion syndromes, mitochondrial encephalomyopathy, lactic acidosis and stroke-like episodes (MELAS), mitochondrial neurogastrointestinal encephalomyopathy (MNGIE), myoclonus epilepsy with ragged red fibers (MERRF), neuropathy, ataxia and retinitis pigmentosa (NARP), Pearson syndrome or progressive external opthalmoplegia (PEO). The nucleic acid therapy may be for treating a congenital myopathy, such as a cap myopathy, a centronuclear myopathy, a congenital myopathies with fiber type disproportion, a core myopathy, a central core disease, a multiminicore myopathies, a myosin storage myopathies, a myotubular myopathy, or a nemaline myopathy. The nucleic acid therapy may be for treating a distal myopathy, such as GNE myopathy/Nonaka myopathy/hereditary inclusion-body myopathy (HIBM), Laing distal myopathy, Markesbery-Griggs late-onset distal myopathy, Miyoshi myopathy, Udd myopathy/tibial muscular dystrophy, VCP Myopathy/IBMPFD, vocal cord and pharyngeal distal myopathy, or Welander distal myopathy. The nucleic acid therapy may be for treating an endocrine myopathy, such as hyperthyroid myopathy or hypothyroid myopathy. The nucleic acid therapy may be for treating an inflammatory myopathy such as dermatomyositis, inclusion body myositis, or polymyositis. The nucleic acid therapy may be for treating a metabolic myopathy, such as Acid maltase deficiency (AMD, Pompe disease), carnitine deficiency, carnitine palmitoyltransferase deficiency, debrancher enzyme deficiency (Cori disease, Forbes disease), lactate dehydrogenase deficiency, myoadenylate deaminase deficiency, phosphofructokinase deficiency (Tarui disease), phosphoglycerate kinase deficiency, phosphoglycerate mutase deficiency, or phosphorylase deficiency (McArdle disease). The nucleic acid therapy may be for treating a myofibrillar myopathy, or a scapuloperoneal myopathy. The nucleic acid therapy may be for treating a neuromuscular junction disease, such as congenital myasthenic syndromes (CMS), Lambert-Eaton myasthenic syndrome (LEMS), or myasthenia gravis (MG). The nucleic acid therapy may be for treating a peripheral nerve disease, such as Charcot-Marie-Tooth disease (CMT), or giant axonal neuropathy (GAN).

The nucleic acid therapy may be for treating a cardiovascular disease such as Thromboangiitis obliterans/Buerger disease, diabetic peripheral neuropathy (also tested in ALS, critical limb ischemia and foot ulcers), peripheral artery disease, limb ischemia, critical limb ischemia (also known as chronic limb threatening ischemia and diabetic limb ischemia), severe peripheral artery occlusive disease (PAOD), or intermittent claudication/arteriosclerosis. For example, the nucleic acid may encode one or more of the transgenes hepatocyte growth factor (HGF), vascular endothelial growth factor (VEGF) and/or Fibroblast growth factor (FGF). In particular, the transgenes hepatocyte growth factor (HGF), vascular endothelial growth factor (VEGF) and/or Fibroblast growth factor (FGF) may be useful in the treatment of a limb ischemia, such as diabetic limb ischemia, in a subject.

The nucleic acid therapy may be for treating an infectious disease, such as COVID-19, HIV, HBV, HCV, Ebola and Marburg virus, West Nile fever, SARS, avian flu, HPV, cytomegalovirus, or malaria. The nucleic acid therapy may be for treating a cancer, such as a sarcoma, melanoma, breast cancer, lung cancer, pancreatic cancer, prostate cancer, liver cancer, acute myeloid leukaemia or B-cell lymphoma. The nucleic acid therapy may be for treating an allergy, such as peanut allergy. The nucleic acid therapy may be for treating multiple sclerosis (MS). The nucleic acid therapy may be for treating myelodysplastic syndrome (MDS).

Pompe disease results from a defect in human acid α-glucosidase (GAA), a lysosomal enzyme that cleaves terminal α1-4 and α1-6 glucose from glycogen. The composition of the invention may be used to treat Pompe disease. The composition of the invention, comprising a nucleic acid that encodes GAA, may be administered to a subject that suffers from Pompe disease in order to deliver the nucleic acid to target tissue of the subject, to express the GAA in a target tissue described herein, particularly the liver and skeletal muscle. The enzyme may be secreted from tissues into the circulation.

Follistatin is an inhibitor of TGF-β superfamily ligands that repress skeletal muscle growth and promote muscle wasting. The composition of the invention, comprising a nucleic acid that encodes follistatin, may be administered to a subject that suffers from a muscle wasting disorder in order to deliver the nucleic acid to target tissue of the subject, to express the follistatin in a target tissue described herein, particularly the liver and skeletal muscle. The protein may be secreted from tissues into the circulation. They may be included in compositions, including a pharmaceutical composition comprising the nanoparticle and a pharmaceutically acceptable excipient. The pharmaceutical composition may be used in medicine. The pharmaceutical composition may be for use in the treatment of a cancer, an autoimmune disease, a lung disease and/or a myopathy. Also described is a method of treating a cancer, an autoimmune disease, a lung disease and/or a myopathy, wherein the method comprises characterising and administering the pharmaceutical composition to a patient or subject. In some embodiments, the composition or pharmaceutical composition is comprised within a liquid. In other embodiments, the composition or pharmaceutical composition is provided as a dry composition, e.g. a dry powder. The dry composition may be prepared using lyophilisation and/or freeze-drying techniques.

Current immunotherapy response rates are around 15-20%, there is an urgent need to improve the treatment outcome. One strategy is to deliver mRNA to express proteins (such as CEBPA, IRF5, IRF8, cGAS-STING, SOCS1 and/or SOCS3) to revert the immunosuppressive phenotypes of myeloid cells in the tumour microenvironment, which would provide a more favourable environment for immunotherapy to be responsive. Another strategy can be to transfer mRNA into the immune cells in the tumour to express cytokines (such as IL-2, IL-7, IL-12, IL-15, IL-21 and/or interferon) to activate the immune cells to fight against cancer cells. It is also possible to deliver mRNA to the macrophage to express a chimeric antigen receptor so that the macrophage can be activated to kill tumour cells. Delivering mRNA to express tumour antigens in antigen presenting cells would help activate the immune system to attack cancer cells. These strategies can be applied to treat all tumours, especially Non-small cell lung cancers or small cell lung cancer (SCLC), Advanced melanoma, Prostate cancer, Ovarian cancer, Breast cancer, Lung cancer, Bile duct cancer (Cholangiocarcinoma), Gallbladder cancer, Neuroendocrine tumours, Hepatocellular carcinoma, Colorectal cancer, Pancreatic cancer, liver cancer, prostate cancer, thyroid cancer, a pancreatic cancer such as pancreatic ductal adenocarcinoma (PDAC), acute myeloid lymphoma (AML), myelodysplastic syndromes (MDS), colorectal cancer such as WT KRAS CRC, a KRAS mutated metastatic tumour, a haematological tumour, an oesophageal cancer, breast cancer, prostate cancer, bladder cancer, a tumour of the GI tract, head and neck squamous cell carcinomas (HNSCC), renal cancer, myelofibrosis, a CD206+ cancer, melanoma and Solid tumours.

The compositions of the disclosure can be used to treat lung diseases such as cystic fibrosis, asthma, tuberculosis (TB), acute lung injury (ALI), pulmonary fibrosis, such as idiopathic pulmonary fibrosis, allergic airway disease, chronic obstructive lung disease (COPD), fibrotic lung disease, chronic lung disease or a respiratory tract infection. The compositions of the disclosure can be used to treat muscle diseases such as a muscular dystrophy or a muscle wasting disease. The compositions of the disclosure can be used to treat kidney disease. The compositions of the invention can be used to treat rare genetic diseases (in haematology, neurology, amyloidosis, pulmonology, endocrinology and nephrology). The compositions of the invention can be used to deliver anti-infectives (for instance, to target macrophages to kill bacteria/deliver antibiotic payload).

Thus, this invention provides methods for treating such disorders, and methods of providing compositions for use in such treatments (and generally methods of providing any compositions described herein, including but not limited to therapeutic/pharmaceutical compositions), comprising characterising a nanocarrier included in said compositions according to a method as described herein.

The nucleic acid-containing compositions of the disclosure can be stored and administered in a sterile pharmaceutically acceptable carrier. Various sterile solutions may be used for administration of the composition, including water, PBS, TRIS buffers, HEPES buffers, ethanol, lipids, etc. The concentration of the DNA/RNA will be sufficient to provide a therapeutic dose, which will depend on the efficiency of transport into the cells.

The compositions of the disclosure can be used to deliver the nucleic acids described herein to certain tissues of the human or animal body. For instance, delivery to skeletal muscle, liver, lung, heart, white and/or brown adipose tissues, brain, spleen, bone marrow, joints, kidney, gastrointestinal tract, eyes, thymus, skin, lymph nodes, pancreas, adrenal gland, testis, prostate, ovary, uterus, bladder, diaphragm, and tumours is possible.

### Chimeric Antigen Receptors (CARs)

The nanocarriers of the present disclosure may be used for transfecting target cells with DNA/RNA encoding Chimeric antigen receptors (CARs). Target cells for CAR transfection include, but is not limited to, T cells, including γδ-T cells, Macrophages and Natural killer (NK) cells. Examples of CAR constructs envisaged by the invention include an anti-carcinoembryonic antigen (CEA) CAR, an anti-CEA Cell Adhesion Molecule 7 (CEACAM7) CAR and an anti-CEACAM5 CAR.

CARs are a class of recombinant proteins which typically comprise an antigen recognition domain, typically a single chain variable fragment (scFv), a hinge region or ectodomain, transmembrane domain and an intracellular signalling/activation domain. An scFv domain is a chimeric peptide comprising a variable light chain (VL) domain and a variable heavy chain (VH) domain of an immunoglobulin linked together such that the scFv can interact with the target antigen. Other antigen recognition domains may be used in place of scFv domains, for example, TNF receptors, innate immune receptors, cytokines, structure protein, and growth factors.

The hinge region, also known as an ectodomain or spacer, is present between the antigen recognition domain and the transmembrane domain. Ideally, the hinge region will lack FcγR binding activity. The hinge may be derived from an IgG, for example the hinge may comprise the CH2 and CH3 of an lgG. Alternatively, the hinge region may be derived from CD28, CD8α which naturally lack FcγR binding activity.

The transmembrane domain is present between the hinge region and the intracellular signalling domain. Any suitable transmembrane domain may be used in a CAR. Typically, the transmembrane domain is derived from CD3-ξ, CD4, CD8 or CD28.

The intracellular signalling domain of a CAR typically comprises a CD3-zeta cytoplasmic domain as the main intracellular signalling domain. In addition, a CAR will normally also comprise one or more co-stimulatory domains. The co-stimulatory domains may be derived from CD27, CD28, CD134 and CD137.

Binding of the antigen recognition domain of a CAR to its target antigen causes the clustering CARs. This clustering results in the transmission of an activation signal for the intracellular T-cell signalling domain, which in turn activates intracellular signalling pathways to stimulate the desired biological response.

The CAR platform was first described in T-cell based immunotherapies (CAR-T) and have been shown to successfully treat a number of haematological cancers. More recently, the CAR platform has been extended to other leukocytes such as CAR-expressing NK (CAR-NK) and γδ-T cells (CAR-γδT). The CAR platform has also been extended to myeloid cells including CAR-expressing macrophages (CAR-M) which are particularly useful at targeting and treating solid tumours.

### Gene Editing

The nanocarriers of the present disclosure may be used in gene editing therapies, including gene editing therapies using technologies those well known in the art such as CRISPR/Cas (e.g. CRISPR/Cas9 systems), TALENS and Zinc finger nucleases.

In some embodiments, the CRISPR/Cas system comprises a Cas nuclease, a crispr RNA (crRNA) and a trans-activating crRNA (trRNA or tracrRNA). In this system, the crRNA comprises a sequence complementary to the target DNA and serves to direct the Cas nuclease to the target site in the genome and the tracrRNA serves as a binding scaffold for the Cas nuclease which is required for Cas activity. In some embodiments, the CRISPR/Cas system comprises a Cas nuclease and a single-guide RNA (sgRNA) to direct the Cas nuclease to the target site in the target gene. An sgRNA comprises a target-specific crRNA fused to a scaffold tracrRNA in a single nucleic acid.

In some embodiments, the nucleic acid comprises a DNA or an mRNA encoding a Cas protein or peptide, for example a Cas9 protein or peptide. In some embodiments, the nucleic acid comprises an sgRNA. In some embodiments, the nucleic acid comprises a crRNA and/or a tracrRNA. In some embodiments, the nucleic acid comprises a DNA or mRNA encoding a Cas protein or peptide, a crRNA and a tracrRNA. In some embodiments, the nucleic acid comprises a DNA or mRNA encoding a Cas protein or peptide and a sgRNA.

The CRISPR/Cas system can also be used to direct repair or modification of a target gene. For example, the CRISPR/Cas system can include a nucleic acid template to promote DNA repair or to introduce an exogenous nucleic acid sequence into the target gene by, for example, promoting homology directed repair. The CRISPR/Cas system may also be used to introduce a targeted modification to the target genomic DNA, for example using base editing technology. This can be achieved using Cas proteins fused to a base editor, such as a cytidine deaminase, as disclosed in, for example, WO2017070633A2 which is incorporated by reference. In another example, the CRISPR/Cas system may be used to “rewrite” a nucleic acid sequence in a genome. For example, the CRISPR/Cas system may be a Prime editing system. In such a prime editing system, a fusion protein may be used. For example, the fusion protein may comprise a catalytically impaired Cas domain (e.g. a “nickase”) and a reverse transcriptase. The catalytically impaired Cas domain may be capable of cutting a single strand of DNA to produce a nicked DNA duplex. A Prime editing system may include a prime editing guide RNA (pegRNA) which includes an extended sgRNA comprising a primer binding site and a reverse transcriptase template sequence. Upon nicking of the DNA duplex by the catalytically impaired Cas, the primer binding site allows the 3′ end of the nicked DNA strand to hybridize to the pegRNA, while the RT template serves as a template for the synthesis of edited genetic information.

In some embodiments, the CRISPR/Cas gene editing system may include a nucleic acid template to direct repair of the target gene of interest. In other embodiments, the Cas protein or peptide may include a base editor. In still further embodiments, the CRISPR/Cas system may be a prime editing system.

### CRISPR/Cas Gene Silencing and Gene Activation

CRISPR/Cas systems have been adapted for use in gene silencing and activation. Such systems are envisaged for use with the current invention. For example, in some embodiments, the nucleic acid may encode a fusion protein comprising a Cas protein or peptide fused to a transcriptional repressor or activator. In some embodiments, the Cas protein is catalytically dead. The fusion protein may be directed to a site of interest in the genome by either an sgRNA or a crRNA. On binding of the fusion protein to the site of interest, the transcriptional repressor or activator can regulate the expression of a gene of interest.

### Nucleic Acid-Based Vaccines

DNA vaccines, as defined by the World Health Organisation (WHO), and RNA vaccines involve the direct introduction into appropriate tissues (of the subject to be vaccinated) a plasmid containing the DNA sequence or RNA encoding the antigen(s) against which an immune response is sought, and relies on the in situ production of the target antigen. These approaches offer a number of potential advantages over traditional approaches, including the stimulation of both B- and T-cell responses, improved vaccine stability, the absence of any infectious agent and the relative ease of large-scale manufacture. As proof of the principle of DNA vaccination, immune responses in animals have been obtained using genes from a variety of infectious agents, including influenza virus, hepatitis B virus, human immunodeficiency virus, rabies virus, lymphocytic chorio-meningitis virus, malarial parasites and mycoplasmas. In some cases, protection from disease in animals has also been obtained. However, the value and advantages of DNA vaccines must be assessed on a case-by-case basis and their applicability will depend on the nature of the agent being immunized against, the nature of the antigen and the type of immune response required for protection.

The field of DNA and RNA vaccination is developing rapidly. Vaccines currently being developed use not only DNA, but also include adjuncts that assist DNA to enter cells, target it towards specific cells, or that may act as adjuvants in stimulating or directing the immune response. As of 2020, the WHO noted that the first nucleic acid vaccines licensed for marketing were likely to use plasmid DNA derived from bacterial cells, but that, in future, others may use RNA or may use complexes of nucleic acid molecules and other entities. However, with the onset of the COVID-19 pandemic in 2020, a concerted effort was made to bring the first RNA-based, COVID-19 vaccines to market and these were approved for use in mid- to late-2020. Since approval, these RNA-based vaccines have been successfully rolled out worldwide to immunise the population against COVID-19.

Intramuscular delivery of DNA vaccines, in common with other vaccine technologies, is a common approach (Lim et al, 2020). The low replication rate of myocytes (muscle cells) in the skeletal muscle makes this an attractive target for DNA vaccination, because stable expression does not rely on genomic integration.

The RNA vaccines on the market currently use mRNA encoding the antigen as a payload. An area now being explored to increase the effectiveness of RNA vaccines is the use of self-amplifying RNA. self-amplifying RNA shares many of the structural features of mRNA and may include a 5′ cap, 3′ polyA tail and 5′ and 3′ untranslated regions (UTRs). In addition to encoding the antigen of interest a self-amplifying RNA will also comprise a system for self-amplification. For example, a self-amplifying RNA may also encode an RNA-dependent RNA polymerase (RDRA), a promoter and the antigen of interest. Upon translation of an RDRA by the subjects translation machinery, the RDRA can engage the self-amplifying RNA and replicate the RNA. Including a system for self-amplification reduces the minimal RNA required in a vaccine and as a result will reduce the likelihood of a subject experiencing side effects.

### Combination Therapies

Compounds characterised by methods of the present disclosure may be used in the treatment of tumours and cancer in subjects in need of treatment thereof. The compounds may be administered alone or in combination with other anticancer agents.

An “anticancer agent” refers to any agent useful in the treatment of a neoplastic condition. One class of anti-cancer agents comprises chemotherapeutic agents. “Chemotherapy” means the administration of one or more chemotherapeutic drugs and/or other agents to a cancer patient by various methods, including intravenous, oral, intramuscular, intraperitoneal, intravesical, subcutaneous, transdermal, buccal, or inhalation or in the form of a suppository. Some chemotherapeutic agents are cytotoxic.

Cytotoxic chemotherapeutic agents trigger cell death via mechanisms or means that are not receptor mediated. Cytotoxic chemotherapeutic agents trigger cell death by interfering with functions that are necessary for cell division, metabolism, or cell survival. Because of this mechanism of action, cells that are growing rapidly (which means proliferating or dividing) or are active metabolically will be killed preferentially over cells that are not. The status of the different cells in the body as dividing or as using energy (which is metabolic activity to support function of the cell) determines the dose of the chemotherapeutic agent that triggers cell death. Cytotoxic chemotherapeutic agents non-exclusively relates to alkylating agents, anti-metabolites, plant alkaloids, topoisomerase inhibitors, antineoplastics and arsenic trioxide, carmustine, fludarabine, IDA ara-C, myalotang, GO, mustargen, cyclophosphamide, gemcitabine, bendamustine, total body irradiation, cytarabine, etoposide, melphalan, pentostatin and radiation. Ibrutinib (BTK inhibitor) is another anticancer agent that can be used in combination with medical applications of this invention. BTK inhibitors enhance TAM repolarisation to M1 phenotype. This combination therapy may be particularly useful for treating solid tumours, particularly ‘cold’ tumours e.g. PDAC.

Anticancer agents also include protein kinase inhibitors which can be used in the treatment of a diverse range of cancers, including blood and lung cancers. Protein kinases typically promote cell proliferation, survival and migration and are often constitutively overexpressed or active in cancer. Inhibitors of protein kinases are therefore a common drug target in the treatment of cancers. Examples of kinase inhibitors for use in the clinic include Crizotinib, Ceritinib, Alectinib, Brigatinib, Bosutinib, Dasatinib, Imatinib, Nilotinib, Ponatinib, Vemurafenib, Dabrafenib, Ibrutinib, Palbociclib, Sorafenib, and Ribociclib.

Anticancer agents also include agents for use in immunotherapy, including antibodies. Immunotherapies can elicit, amplify, reduce or suppress an immune response depending on the specific disease context. For example, tumour cells expressing the PDL1 ligand suppress the normal immune response in a subject by binding to PD-1 receptor expressed on T cells. In this way, tumour cells resist immunity-induced apoptosis and promote tumour progression. Anti-PD-1 and anti-PDL1 antibodies have been employed successfully in the clinic to inhibit this immune checkpoint and promote immune cell-mediated killing of tumour cells. Other examples of immunotherapy include oncolytic viral therapies, T-cell therapies, and cancer vaccines.

### Pharmaceutical Compositions

As used herein, a pharmaceutical compositions may include one or more pharmaceutically acceptable excipients or carriers, e.g., solvents, solubility enhancers, suspending agents, buffering agents, isotonicity agents, antioxidant, antimicrobial preservatives, diluents, binders, lubricants and disintegrants. “Pharmaceutically acceptable” refers to molecular entities and compositions that are “generally regarded as safe”, e.g., that are physiologically tolerable and do not typically produce an allergic or similar untoward reaction, such as gastric upset and the like, when administered to a human.

In some embodiments, this term refers to molecular entities and compositions approved by a regulatory agency of the US federal or a state government, as the GRAS list under section 204 (s) and 409 of the Federal Food, Drug and Cosmetic Act, that is subject to premarket review and approval by the FDA or similar lists, the U.S. Pharmacopeia or another generally recognised pharmacopeia for use in animals, and more particularly in humans.

When used, the excipients of the compositions will not adversely affect the stability, bioavailability, safety, and/or efficacy of the active ingredients. Thus, the skilled person will appreciate that compositions are provided wherein there is no incompatibility between any of the components of the dosage form. Excipients may be selected from the group consisting of buffering agents, tonicity agents, chelating agents, antioxidants, antimicrobial agents, and preservatives.

### Expression of Therapeutic Products

A nucleic acid that is delivered by a nanocarrier characterised according to the disclosure may exhibit a therapeutic action (e.g. by acting directly to down or up regulate a target gene) or it may express a gene product (which could be a therapeutic protein or therapeutic nucleic acid) via an expression cassette comprising a coding sequence operably linked to a promoter. In this specification the term “operably linked” may include the situation where a selected nucleotide sequence and regulatory nucleotide sequence are covalently linked in such a way as to place the expression of a coding sequence under the influence or control of the regulatory sequence. Thus, a regulatory sequence is operably linked to a selected nucleotide sequence if the regulatory sequence is capable of effecting transcription of a coding sequence which forms part or all of the selected nucleotide sequence. Where appropriate, the resulting transcript may then be translated into a desired protein or polypeptide.

### Route of Administration

The compositions according to aspects of the present invention may be formulated for administration by a number of routes, including but not limited to, intravenous, parenteral, intra-arterial, intramuscular, intratumoural, subcutaneous, oral and nasal.

Actual delivery of the composition of the invention to a subject (human or animal) can be carried out by a variety of techniques including direct injection, instillation of lung and other epithelial surfaces, or by intravenous parenteral, intra-arterial, intramuscular, intratumoural, or subcutaneous injection. Administration may be by syringe needle, trocar, cannula, catheter, etc, as a bolus, a plurality of doses or extended infusion, etc. It is also envisaged that a nucleic acid cargo may be delivered to patient cells or donor cells ex vivo prior to perfusion of the patient or donor cells into a subject.

### Subject

A subject to be treated may be any animal or human. The subject is preferably mammalian, more preferably human. The subject may be a non-human mammal, but is more preferably human. The subject may be male or female. The subject may be a patient. Therapeutic uses may be in human or animals (veterinary use).

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example+/−10%.

### EXAMPLES

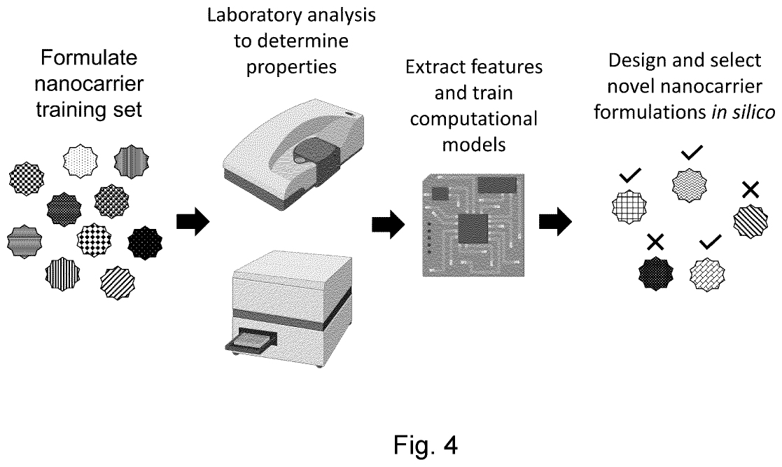

In these examples, the inventors demonstrate computational methods, models, and systems for predicting the structural attributes and transfection performance of nanocarriers designed to deliver genetic payloads to human cells. They evaluated the predictive performance of the models obtained and show the capacity of the computational system to guide novel nanocarrier design using real-world experimental validation. Further, they establish a method that can be applied to any predictive computational model to discover the most important contributing attributes that influence nanocarrier properties of interest. The process followed in these examples is summarised in FIG. 4. First, the inventors formulated a nanocarrier training set, which was then analysed to determine a plurality of properties of the nanocarriers. A feature extraction and machine learning model training process were then applied to the training data set, resulting in trained models that can be used to design and select novel nanocarrier formulations in silico.

### Example 1—Data Acquisition

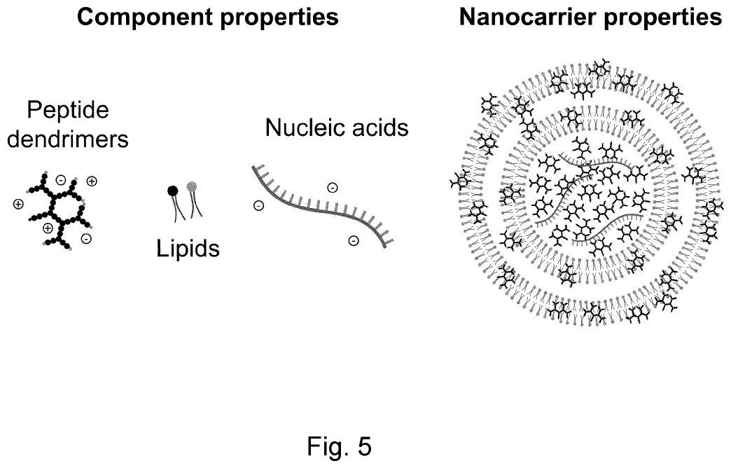

Nucleic acid delivery systems based on cationic lipids are one of the most studied and efficient non-viral vector platforms described to date, and the rational design and development of peptidic vectors with natural amino acids are particularly attractive for therapeutic applications due to the non-toxic nature of the amino acids. The inventors have developed a structural framework for nucleic acid delivery, using peptide dendrimers, illustrated on FIG. 5. Note that the structure illustrated on FIG. 5 is for illustrative purposes only and the exact structure of the nanoparticles may vary, for example depending on ratios of the components and charge distribution. The nanocarriers used in the present examples comprise positively charged peptide dendrimers and one or more lipids interacting with and packaging a negatively charged nucleic acid cargo. The structural framework for the peptide dendrimers used in the present examples involves layers of peptide (or dipeptide) motifs, bound to lysine residues. The inventors have found that the distribution of cationic amino acid residues (Lys or Arg) in each generation (layer) gave peptide dendrimers transfecting more efficiently than dendrimers with charges localized solely on the surface (Kwok et al, 2013). Using a solid phase peptide dendrimer synthetic procedure, the inventors can precisely manipulate the position of every amino acid residue incorporated within the dendritic scaffold. This allows greater control of the structure and function of the dendrimer, which was normally not possible with previously studied systems such as polymers or other dendrimers where modifications were mainly made on the surface of the molecule. The peptide dendrimer/lipid vector show high transfection efficiency, good reproducibility of results and low toxicity.

In this example, such vectors are formulated and tested according to the procedures described below.

Cell lines, transfection reagents and mRNA. Hela cells were maintained in RPMI medium with 10% (v/v) FCS and 1% (v/v) L-glutamine in a humidified atmosphere in 5% CO2 and 37° C. The eGFP mRNA was purchased from Trilink (CleanCap® EGFP mRNA (5moU)-(L-7201)). DOTMA:DOPE, 1:1 (w/w) were obtained from Invitrogen (Lipofectin™ Transfection Reagent) (Fisher-18292037).

Nanoparticle formulation procedure. To formulate the dendrimer nanoparticle, the dendrimers were used in solutions of 10 mg/ml in water or 0.5 mg/ml in water. The mRNA was used as a 65 μl batch at 50 μg/ml mRNA.

Tube A (Dendrimer): to a sterile 1.5 ml polypropylene tube, 50 mM HEPES buffer (3.25 μl), sterile water (to give a final volume of 6.50 μl) and 10 mg/ml dendrimer stock solution (7.879×10-5 mmoles N) was added. For an N/P ratio of 0.15625, a 0.5 mg/ml dendrimer stock solution (1.539×10-6 mmoles N) was used. The quantities of dendrimer stock solution and water vary depending on the molecular weight and charge of the dendrimer. Tube B (200 μg/ml mRNA in 25 mM HEPES buffer): to a sterile 1.5 ml polypropylene tube, 200 mM HEPES buffer (134.1 μl), sterile water (724.00 μl) and 1 mg/ml mRNA stock solution (214.50 μl) were added, and the tube was mixed with gentle shaking. Tube C (769 μg/ml lipofectin in 25 mM HEPES buffer): to a sterile 15 ml polypropylene falcon tube, 200 mM HEPES buffer (348.60 μl), sterile water (294.9 μl) and 1 mg/ml Lipofectin™ solution (2.145 ml) were added, and the tube was mixed with gentle shaking. Mixing: 200 μg/ml mRNA solution (16.26 μl, 3.252 μg, 9.855×10-6 mmoles P) was mixed into tube A by rapidly pipetting up and down 10 times. This was allowed to sit for 5 minutes. 769 μg/ml lipofectin solution (42.20 μl, 32.46 μg) was mixed into tube A by rapidly pipetting up and down 10 times.

A variety of lipid-peptide dendrimer nanocarrier formulations were included in the training data set used in the examples below. These were formulated using a process as described below for an exemplary peptide of the training dataset.

Example peptide 1: RHCG1-R or (R)2KRHC-NH2 (described in more detail in WO 2022/162200A1, see e.g. page 11).

### Example Formulations

Example peptide 1, N/P ratio 8 (1538 g/mol, 5 charges per dendrimer): Tube A (Dendrimer): to a sterile 1.5 ml polypropylene tube, 25 mM HEPES buffer (3.25 μl), sterile water (0.826 μl) and 10 mg/ml dendrimer stock solution (2.424 μl, 24.2 μg, 1.58×10−5 mmoles dendrimer 7.88×10-5 mmoles N) was added

Example peptide 1, N/P ratio 0.15625 (1538 g/mol, 5 charges per dendrimer): Tube A (Dendrimer): to a sterile 1.5 ml polypropylene tube, 25 mM HEPES buffer (3.25 μl), sterile water (0.826 μl) and 0.5 mg/ml dendrimer stock solution (0.948 μl, 0.474 μg, 3.08×10-5 mmoles dendrimer, 1.54×10-6 mmoles N) was added.

Measurement of structural attributes. The nanoparticles' sizes in nm, zeta potential and polydispersity index (PDI, also labelled as “PdI”) were measured on a ZetaSizer Pro (Malvern Panalytical).

The hydrodynamic size was measured using the dynamic light scattering (DLS) technique using the Zetasizer Advance Series-Pro (Malvern Panalytical Ltd, Malvern, UK) according to the manufacturer's instructions. DLS is a very sensitive, non-invasive method to measure size and size distribution of nanoparticles in a liquid. The Brownian motion of nanoparticles in suspension resulted in laser light to be scattered at different intensities. Analysing these intensity fluctuations allows us to calculate the velocity of the Brownian motion. The size of the nanoparticles can be determined by using the Stokes-Einstein relationship. With the latest technology, it can measure nanoparticles smaller than 1 nm.

The data obtained with DLS measurements can also be used to calculate the PDI of particles in solution. PDI is used to estimate the average uniformity of a particle solution. The method of cumulants is a standard technique for analysing DLS data on sample polydispersity. PDI is a number calculated from a 2-parameter fit to the correlation data (the cumulants analysis). The cumulants analysis is used to evaluate the autocorrelation function generated by a DLS experiment. The calculation is defined in ISO 13321 and ISO 22412. PDI values greater than 0.7 indicate that the sample has a very broad size distribution and is not suitable for the DLS technique. The calculations for these parameters are defined in the ISO standard document 13321:1996 E and ISO 22412:2008.

To measure the size and PDI, the samples were diluted 8× in 25 mM HEPES buffer (10 μl sample+70 μl buffer). Parameters: reference material: polystyrene latex, dispersant: water, 25° C.

Zeta potential is a measurement of the magnitude of the electrostatic or charge repulsion/attraction between particles. This can be determined by analysing particle mobility and charge (Zeta potential) using the Electrophoretic Light Scattering (ELS) technique. To measure the zeta potential, the samples were diluted 47× or 70× in 25 mM HEPES buffer (15 μl sample+685 μl buffer for 47×, or 10 μl sample+690 μl buffer for 70×) and added to a clean DTS1070 cell. Parameters: reference material: polystyrene latex, dispersant: water, 25° C. The measurements of the zeta potential were carried out using the Zetasizer Advance Series-Pro (Malvern Panalytical Ltd, Malvern, UK) according to the manufacturer's instructions.

Transfection procedure. 24 hours before transfection, Hela cells were seeded in 96 well plates in order to reach 70% confluence. mRNA transfection complexes were formed by mixing mRNA with the dendrimers in 25 mM HEPES buffer, then with DOTMA: DOPE in 25 mM HEPES buffer at 25° C. The transfection complexes were then overlaid to the cells in full growth medium. The cells were harvested for reporter gene assay 24-hour post transfection.

Transgene expression assay (eGFP). The cells were washed twice with PBS and incubated with 50 ul of 1× M-PER lysis buffer (Thermo 11874111). Plates were protected from light and gently agitated at RT for 15 minutes to aid cell lysis. 40 ul of lysate from each well was transferred to all black 96 well plates for quantification of eGFP relative fluorescence unit (RFU), absorbance at 535 nm, using Molecular devices SpectraMax iD5.

Protein content determination. The protein content of each cell lysate was determined by mixing the lysate (25 μL) with Pierce™ BCA Protein Assay Kit (200 ul, Thermo Scientific). After 30 minutes of 37° C. incubation in at dark, absorbance was measured at 562 nm with Molecular devices SpectraMax iD5 and converted to protein concentration using a BSA standard curve. RFU per mg of protein represented eGFP expression.

### Example 2—Construction of a Training Data Set—Ground Truth for Predicted Features

In this example, data acquired for a plurality of vectors as described in Example 1 was processed to obtain a training data set for training machine learning models. In particular, the intention was to train machine learning models to be able to predict structural nanocarrier attributes as well as transfection performance.

A total of 165 different nanocarriers were included in the training dataset. All of these were lipid-peptide dendrimer nanocarriers such as e.g. as described in WO2022/162200A1. This training dataset included data for nanoparticles comprising 38 unique dendrimer sequences, at multiple NP ratios.

### Structural Characteristics

For structural nanocarrier attributes the z-average size in nm, PdI, and zeta potentials were obtained using ZS Xplorer v. 1.0.0.436 and used directly as ground truth targets for computational modelling.

For nanocarriers with repeated structural DLS measurements, mean values for size and PdI were taken.

### Transfection Performance

To obtain a ground truth value indicative of transfection performance the inventors processed samples as follows. The eGFP derived RFU of the 6 untreated vehicle technical replicates in each plate was calculated and subtracted from each well in that plate. After background subtraction any experimental wells with RFU less than or equal to 2 standard deviations above background were classified as having failed to transfect the cells in that well. Samples with a greater than 50% technical replicate fail rate (i.e. more than 3 out of 6 wells deemed failed) were excluded from further analysis and classified as unsuccessful transfections. These were still included in the training dataset but assigned a transfection performance value of 0. There were only 8 nanocarriers (out of 165) that were deemed to have failed. Next the protein content of each well was calculated from the absorbance measurements. The median RFU/mg of protein was then taken for each nanocarrier sample where repeated transfection experiments had been conducted.

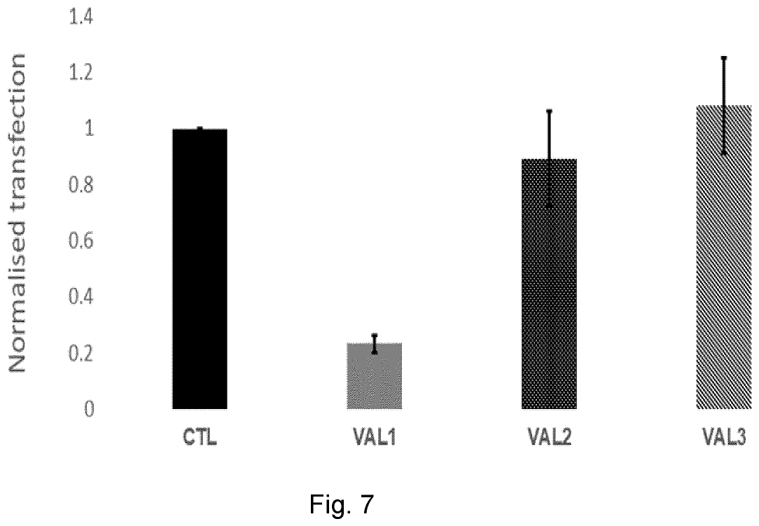

One nanocarrier formulation (control, CTL) that showed a stable size and PdI and had been observed to consistently transfect cells was selected to serve as an internal positive control in each experimental plate. Other nanocarriers' median RFU/mg were divided by this internal control's own RFU/mg to provide a normalised ratio of transfection performance. The inventors validated that normalised transfection performance was consistent from experiment to experiment by performing many rounds of transfection with three well-studied nanocarrier candidates, VAL1, VAL2, and VAL3. 40 replicate experiments were conducted with VAL1, 20 with VAL2, and 20 with VAL3, respectively (FIG. 7). Replicate normalised transfection performance values did not significantly differ from normal distributions (p-value significance threshold 0.05) according to Kolmogorov-Smirnov normality tests (p-value=0.27, 0.21, 0.23, for VAL1, VAL2, and VAL3, respectively), and had a similar spread around their mean value (coefficient of variation=0.45, 0.42, 0.36, for VAL1, VAL2, and VAL3, respectively) demonstrating experimental consistency.

### Results

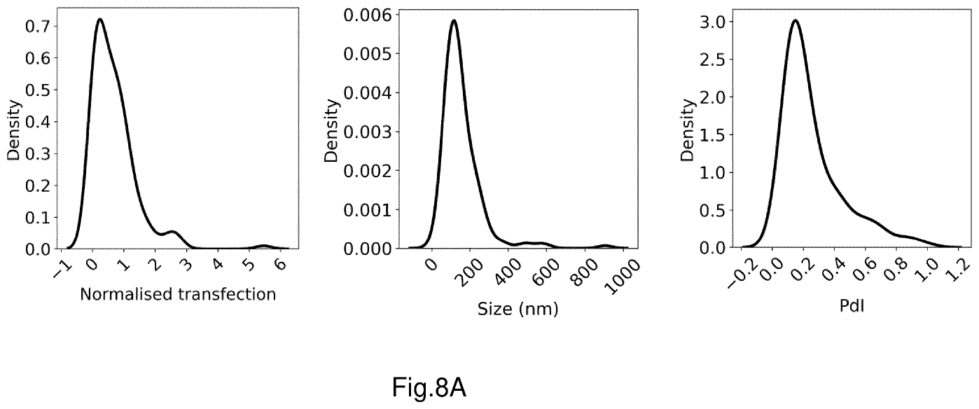

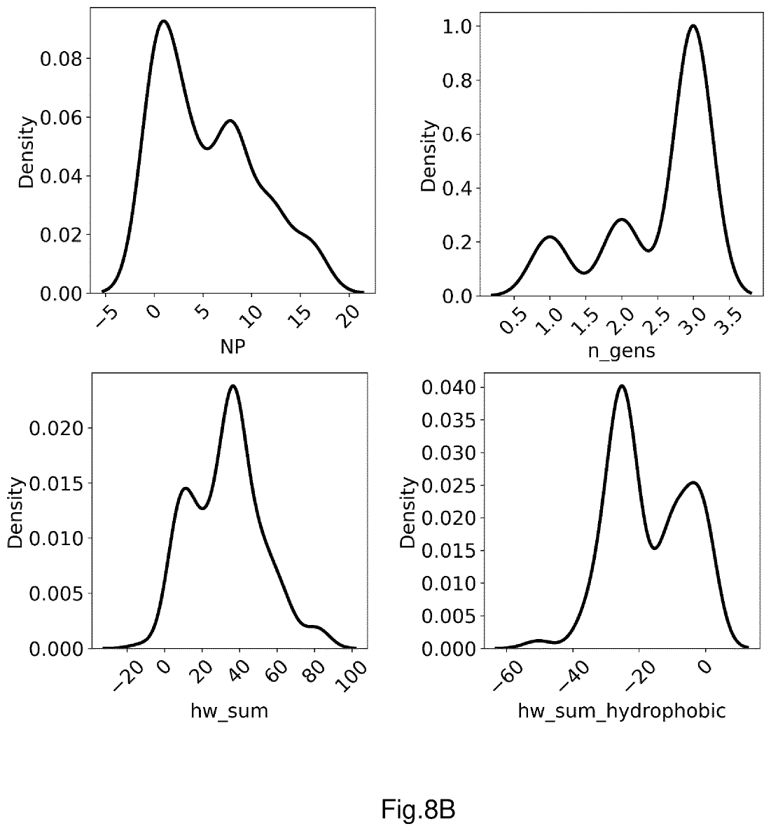

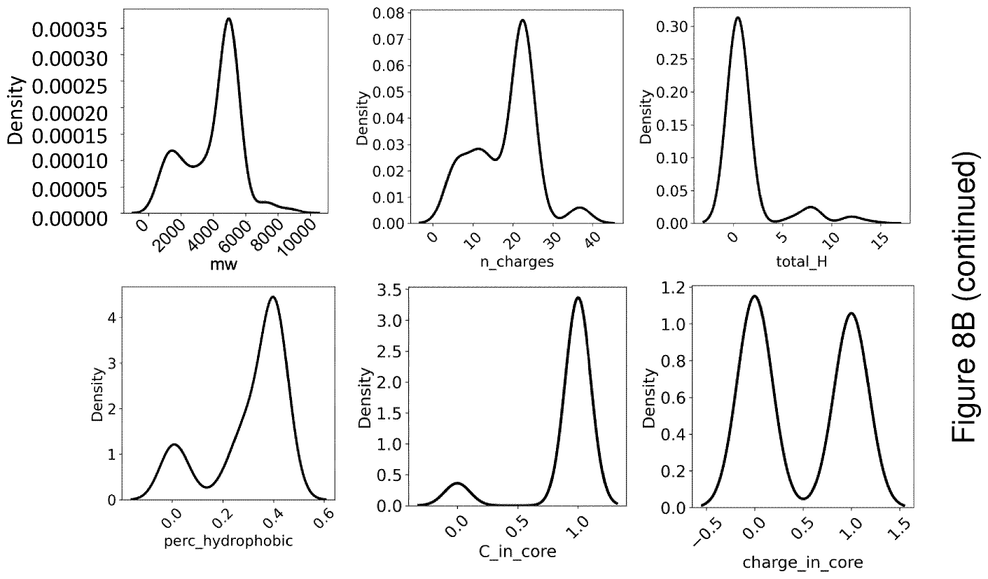

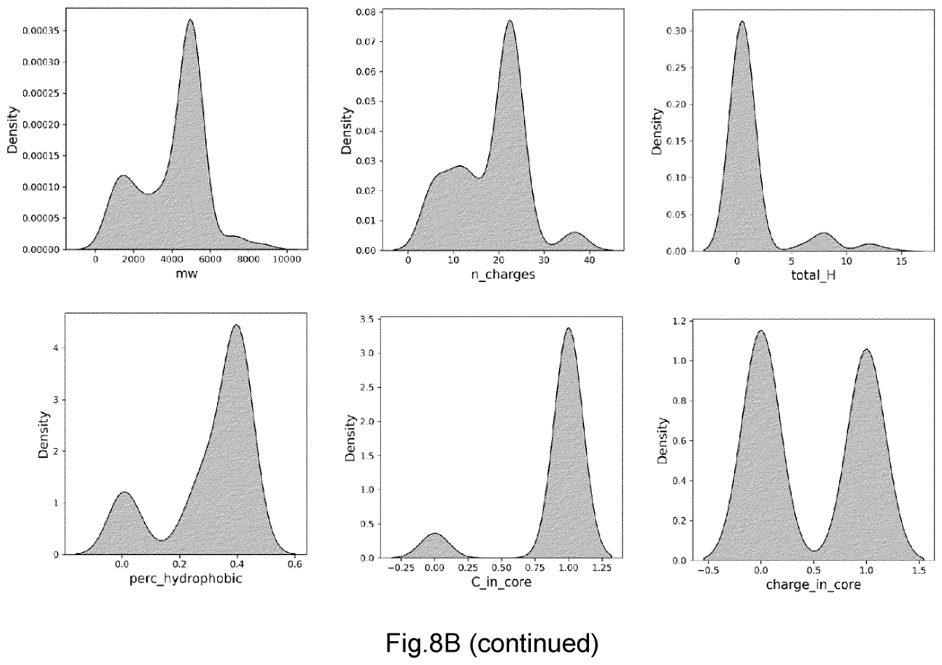

The distributions of measured sizes, PdIs, and normalised transfection performances in the dataset are shown as kernel density estimation (KDE) plots in FIG. 8A. KDE plot bandwidth bw was defined by Scott's rule (log(bw)=−log(n)/(d+4)), where n is the number of examples in the dataset and d is the number of dimensions; in this case d=1 (Scott, 2015). The distributions of values of structural features used for prediction (listed in Example 3 below) across the training data set are shown in FIG. 8B. Note that any set of nanocarriers could have been used as a training data set within the context of the present invention, which is not limited to a training data set comprising 165 different nanocarriers with the features described in FIGS. 8A and 8B. Nevertheless, these figures provide a description of an exemplary dataset that can be used to train a machine learning model according to the present disclosure.

### Example 3—Feature Extraction and Scaling

In this example, predictive features used to train machine learning models based on the training data in Example 2 were defined. The inventors used two classes of input features for our computational models: nanocarrier component features, and dendrimer-specific sequence features.

Nanocarrier features could include, but are not limited to, protein/peptide to payload ratio (e.g. N/P ratio (nitrogen to phosphate, in the case of a nucleic acid cargo)), lipid to payload ratio, lipid type or combinations of lipid types and their ratios, and experimentally measured or predicted attributes such as particle size (nm) and PdI (used as targets in these examples). In the present examples, only the N/P ratio was used as a nanocarrier feature.

Dendrimer-specific sequence features could include, but are not limited to, molecular weight, charge, mass to charge ratio, hydrophilicity or hydrophobicity scores (e.g. as described in Hopp-Woods, 1981 or Kyte-Doolittle, 1982), extinction coefficient, isoelectric point, sequence length, presence/absence of specific residues in the sequence (such as e.g. cysteine and/or histidine), number of branch point or generations in branched peptides. Sequence feature values could be per residue, per generation, or summary values for the entire peptide.

The inventors selected 10 features to generate proof-of-concept models. These are listed in Table 1 along with explanatory descriptions. Following feature crafting, all values were scaled to a similar range by dividing by the maximum possible value for that feature. This was performed primarily to ensure smoother gradients during neural network backpropagation. Note that the minimum value could be subtracted prior to maximum scaling (a procedure known as ‘min-max scaling’).

TABLE 1
Description of input features for machine learning.

| Feature | |
|---|---|
| name | Description |
| NP | The ratio of positively charged amine nitrogen (N) in the |
| | peptide dendrimer (or protein or peptide) to phosphate in |
| | the nucleic acid payload. |
| n_gens | The number of generations comprising the branched |
| | dendrimer. |
| mw | The molecular weight of the dendrimer. |
| n_charges | The number of positively charged sidechains in the |
| | dendrimer peptide sequence. |
| total_H | The total number of histidine residues in the dendrimer |
| | peptide sequence. |
| hw_sum | The sum of the Hopp-Woods hydrophilicity scores from |
| | each residue in the dendrimer. |
| hw_sum_{—} | The sum of the Hopp-Woods hydrophilicity scores from |
| hydrophobic | each hydrophobic residue in the dendrimer. |
| perc_{—} | The percentage of residues comprising the dendrimer |
| hydrophobic | sequence that are classed as hydrophobic. |
| C_in_core | Whether a cysteine residue resides in the dendrimer core |
| | sequence. |
| charge_in_{—} | Where a positively charged side chain is present in the |
| core | dendrimer core sequence. |

The number of positively charged sidechains in the dendrimer peptide sequence was calculated as the number of lysine and arginine residues, not including branching lysines (where the side chain is no longer exposed). The residues classed as hydrophobic for the purpose of calculating hw_sum_hydrophobic and perc_hydrophobic were: F, I, L, M, V, W, Y (i.e. Phe, Ile, Leu, Met, Val, Trp, Tyr).

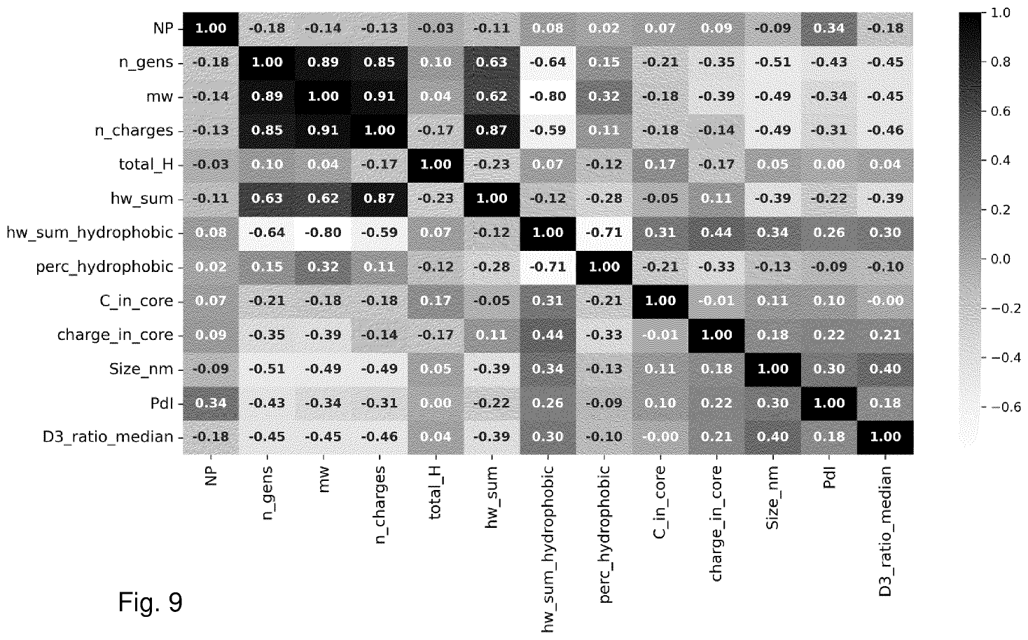

FIG. 9 shows a correlation matrix of the 10 input features along with the 3 proof-of-concept nanocarrier target properties. Although some features show high co-linearity across the full dataset (e.g. mw or n_charges versus n_gens), these features are not completely dependent in practice, as a small heavy peptide is possible and vice versa depending on amino acid composition. A large peptide with very few positively charged side chains is also possible. There was a single significant anticorrelation between the number of generations and the size of the nanoparticle. However, there was no other strong linear association between any of the 10 chosen input features and the target nanocarrier attributes (r>=0.5 or r<=−0.5). Some moderate anticorrelations were observed. This indicates that the target nanocarrier properties cannot be straightforwardly predicted (i.e. without use of machine learning) from the available input features.

Most of the dendrimer sequence features listed in Table 1 are summary properties of the entire dendrimer sequence. Hence, to consider local sequence features and possible hidden structural information about branched peptide dendrimers, the inventors employed learned feature extraction. To prepare inputs for this process, they generated an amino acid residue-level representation of the dendrimer sequence by encoding residue attributes at specific points in a multi-dimensional matrix (3×18×18). Features could then be extracted by a neural network model using convolutional operations (in the present example this is EfficientNetv2 B0 but any deep learning model that can be used for object recognition/computer vision applications may be usable in the present context). The neural network used was one that has been pretrained on an unrelated data set (in this case, RGB images of real-world objects in ImageNet), and hence the use of the same number of channels as in the data used for pre-training (in this case 3) was advantageous. The neural network was then partially retrained by transfer learning. It is also possible for a different number of channels to be used with a pretrained model, for example using a custom convolutional layer after the input to the network to combine or expand channels to the number of channels used by the pretrained model. Alternatively, a neural network that has not been pretrained may be used, if sufficient training data is available. The other dimensions of the multidimensional matrix may be set to any value that is convenient to include all of the desired properties of the component/subcomponent for which learned features are to be predicted. The properties may advantageously be provided as a 2-dimensional matrix as this is equivalent to a 2D image, enabling the use of models pre-trained on image data. For example, in the present case each feature of a peptide dendrimer was provided as an 18×18 pseudo-image (each feature of a particular amino acid in the sequence representing a “pixel” in the pseudo-image). For a proof-of-concept the inventors encoded min-max scaled molecular weight, Hopp-woods hydrophilicity, and cationic charge for individual residues, where:

$${x}^{′} = \frac{x - min ( x )}{( x ) - min ( x )}$$

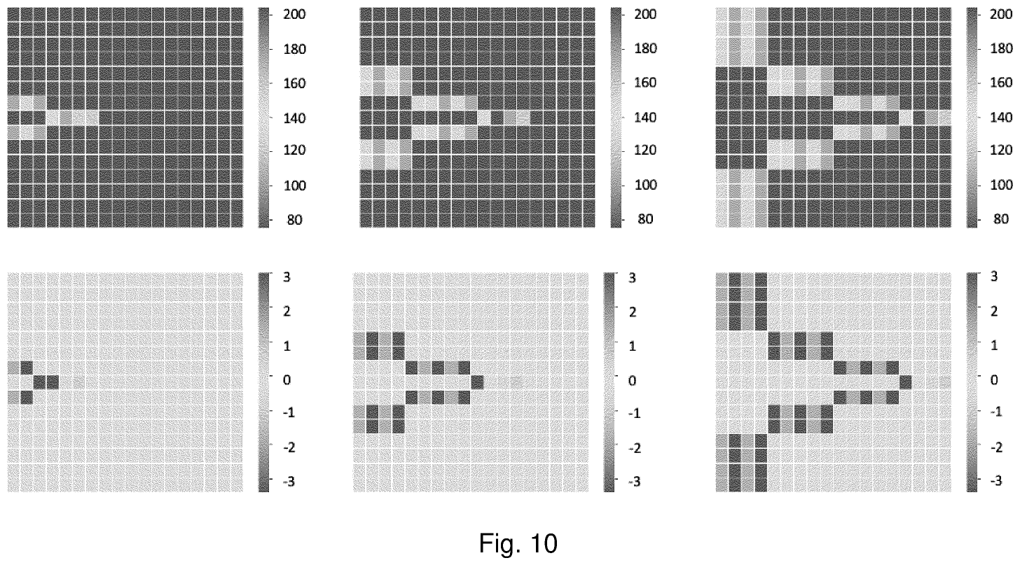

In this case the maximum and minimum values used for normalisation were the highest and lowest values for the 20 naturally occurring amino acids. For quality control and to allow visual inspection of the encodings, the values comprising these can be represented as individual 2-dimensional images. Some examples are shown in FIG. 10 for 1, 2 and 3 generation dendrimers (from left to right, with the top row showing the molecular weight and the bottom row showing the Hopp-Woods hydrophilicity). In this example, the core sequence was encoded starting from the right-hand side of the pseudo-image (although other choices are possible), at the centre of the image. As can be seen on these images, the input matrix is occupied to a different extent depending on the size of the dendrimer (i.e. the third generation dendrimer illustrated occupies the whole horizontal dimension of the images and the whole vertical dimension of the images, while the second and first generation dendrimers have default (0) values for increasing number of pixels of the pseudo-image. Note that these images show unscaled raw molecular weights and hydrophobicity values, but scaled values were used as input to the model. A more advanced encoding approach could be to represent the dendrimer at the atomic level using its underlying chemical structure; this could then be passed as a graph to a graph neural network or represented in 2 or 3 dimensions for input to a convolutional neural network (CNN). For example, 2D or 3D representations of chemical structures (e.g. 2D structures of amino acid sidechains along the sequence, 3D ball and stick representation, full 3D predicted structure, etc.) may be provided as images that are used as input to a model trained to learn features present in images.

### Example 4—Model Training

In this example, the predictive features obtained in Example 3 and the ground truth features obtained in Example 2 were used to train a variety of machine learning (ML) models to predict the structural features and transfection performance of nanocarriers.

Several different computational models were constructed, and their predictive performance compared. In order of increasing complexity, computational models included a naïve random choice (RC) model (to serve as a baseline), a simple ordinary least squares multiple-linear regression (MLR) model, a random forest (RF) machine learning model (Breiman, 2001), an artificial neural network (ANN), and a

CNN. The non-linear ML models (RF, ANN, and CNN) were trained to reduce the mean squared error (MSE):

$$M S E = \frac{1}{n} {\sum }_{i = 1}^{n} {( {y}_{i} - {\hat{y}}_{i} )}^{2}$$

where n is the number of examples, yᵢ is a target value and ŷᵢ is the corresponding predicted value, and n is the number of observations.

As no existing baseline exists in the literature to compare these models to, the inventors created a naïve baseline by constructing a RC model which assigns random values from the training dataset to unseen examples. This ensures that random assignment by the RC model is restricted only to values that are physically possible (those observed to occur in the laboratory). All other models could then be compared to the results of the RC baseline. Given that some moderate linear correlations were observed between calculated input features and target nanocarrier attributes (FIG. 9), the inventors first constructed a linear model to evaluate whether such a simple algorithm could make practically useful predictions. They used ordinary least squares MLR:

$$y = {β}_{0} + {β}_{1} {x}_{1} + {β}_{2} {x}_{2} + ⋯ + {β}_{n} {x}_{n}$$

where y is the target nanocarrier attribute, x₁ to xₙ are the input feature values, β₀ is the computed intercept, and β₁ to βₙ are the slope coefficients for each feature represented on the hyperplane (the β coefficients being learned parameters of the trained model). The MLR could then serve as a secondary benchmark for the non-linear approaches, e.g. to investigate whether the increase in complexity of non-linear approaches was associated with a significant increase in performance.

Once the naïve RC and MLR baselines were obtained, the inventors proceeded to construct more powerful machine learning models that could account for non-linear associations between input features and nanocarrier attributes of interest. Non-linear models included a RF, along with two varieties of deep learning models (ANN, and CNN). The RF model was comprised of 512 estimators (trees). Key hyperparameters were: minimum samples per node split=2, minimum samples per leaf=1. No maximum depth was assigned, so decision trees could expand as required without limits. The MLR and RF models were constructed and trained using scikit-learn 1.0.2 for python 3.

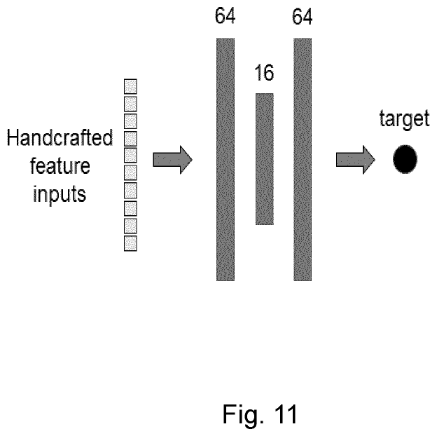

For the ANN, the inventors used a model made up of 3 abstraction layers, comprising 64, 16, and 64 neurons, respectively; each followed by rectified linear unit (ReLU) activation functions (see architecture on FIG. 11). A single output node was used to predict the nanocarrier attribute's target value. Both size and normalised transfection performance were unsigned and unbounded, hence a softplus activation function was utilised after the output node, where:

$$softplus ( x ) = log log ( 1 + exp exp ( x ) )$$

In contrast, PdI values are bounded between 0 and 1, so a sigmoid activation was used for this attribute, where:

$$sigmoid ( x ) = \frac{1}{1 + exp exp ( - x )}$$

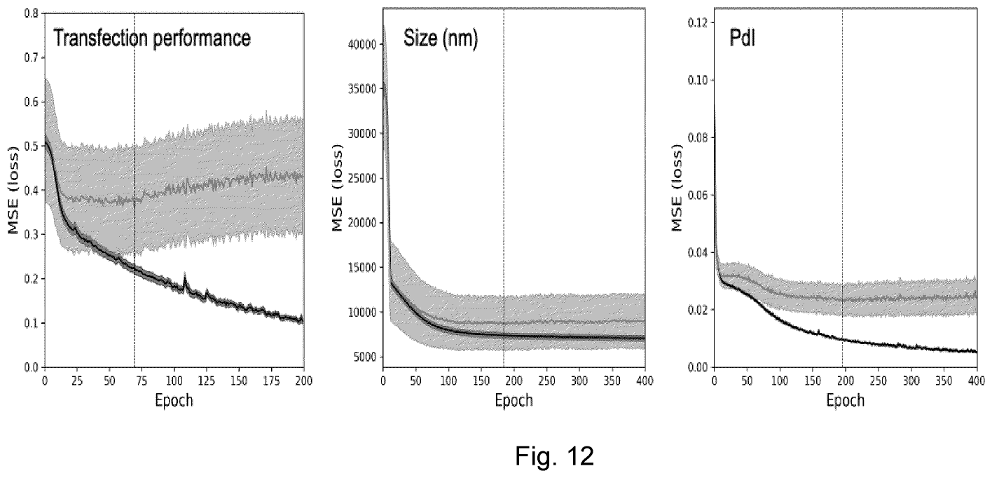

The ANN's trainable weights were initialised with random values from −c to c using a Glorot Uniform initialisation function (Glorot and Bengio, 2010):

$$c = \sqrt{\frac{6}{{n}_{in} + {n}_{out}}}$$

where nᵢₙ is the number of inputs from the previous layer and nₒᵤₜ is the number of outgoing neurons connected to the layer weights. The model was trained using the Adam optimiser (Kingma and Ba, 2014) (β₁=0.9, β₂=0.999, and ε=1e-7) with a learning rate of 1×10⁻³. The inventors used a batch size of 16 examples per step. For each target attribute, to find the best epoch they performed 10-fold cross validation, where the model is trained 10 times with the training set split into 10 equally sized sub-samples. Each time, 9 subsamples are used for training and 1 subsample held-out for validation. They then selected the epoch with the lowest mean validation MSE across all folds. On FIG. 12, the ANN 10-fold cross validation learning curves are shown for each predicted attribute, with the dotted vertical line showing the best epoch, the black line is the training curve and the grey line is the validation curve (average MSE loss±standard error).

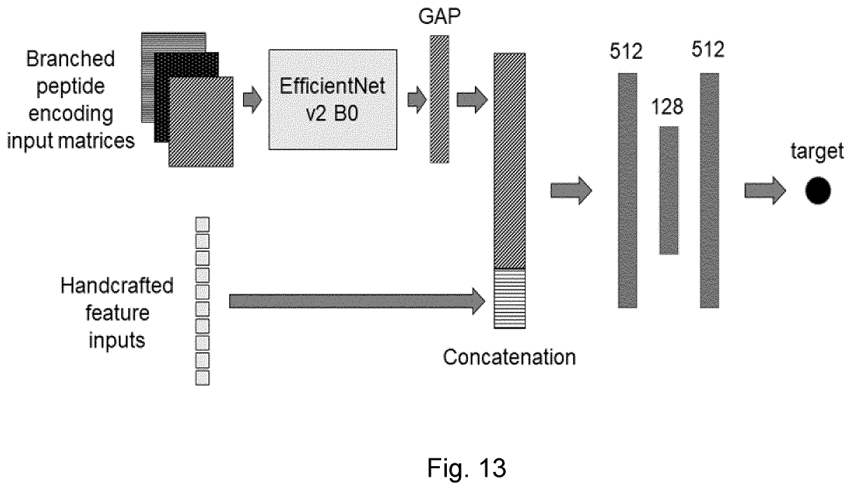

For the CNN, the inventors built a model that accepted 3-channel 224×224 upscaled 2D encoded dendrimer matrices as inputs (see architecture illustrated on FIG. 13). The size of the input data was selected simply for convenience, to match the size of the images on which the CNN had been pretrained.

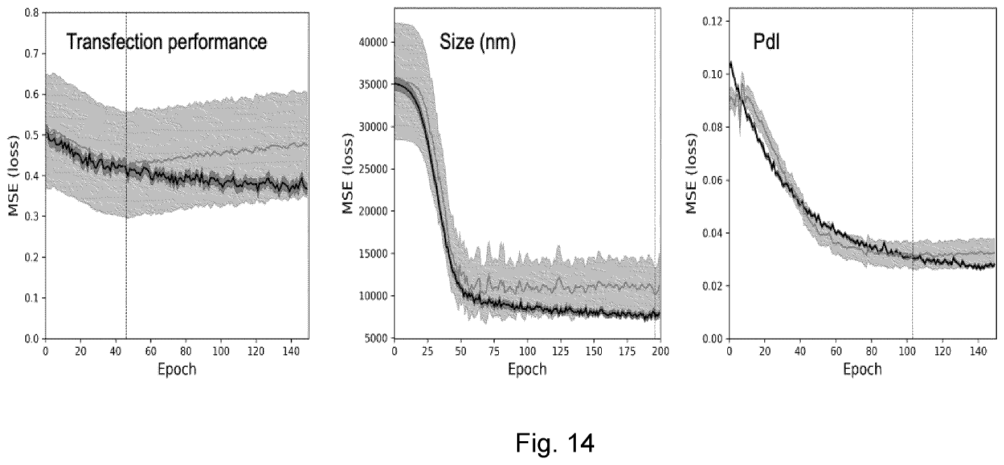

Other options are possible such as e.g. training the network de novo with arbitrary sized inputs. The underlying convolutional network for this proof-of-concept was a baseline EfficientNetv2 BO model pretrained on ImageNet (Tan and Le, 2021), although any CNN could be used for convolutional feature extraction. The network also had access to nanocarrier component features and summary sequence features available to the other models. This was achieved by global average pooling (GAP) and concatenating convolutional features with the 10 summary features after the final convolutional layer. Associations were then learned by feeding the concatenated features forward through a series of dense neural network layers comprising 512, 128, and 512 neurons, respectively, each connected by ReLU activation functions. Like the ANN, the CNN was trained using Adam with a batch size of 16, and with weights in the dense layers initialised using an identical Glorot uniform strategy. However, given the size of the network (over 6.5 million trainable parameters), the CNN was trained with lower learning rates of 1×10-6 (transfection performance or PdI prediction) or 1×10-5 (size prediction). The learning rates for the various models were set based on a combination of default values and trial and error assessing the convergence of models with different learning rates. Again, 10-fold cross validation was used to select the best number of epochs and avoid overtraining (see FIG. 14 which shows the CNN 10-fold cross validation learning curves).

All neural networks were built and trained using TensorFlow 2.9.0 for python 3 and trained on a single Nvidia GTX 3080 (8 GB).

### Example 5—Model Evaluation

In this example, the performance of the models trained in Example 4 was evaluated.

To evaluate each model's performance and for cross-model comparisons, the inventors performed 10-fold cross validation. For the best model obtained in this particular example (RF), they further evaluated overall performance by conducting leave-one-out cross validation (LOOCV), where the model is trained on n−1 training examples n times, and the absolute difference between the held-out example's predicted value and its ground truth computed each time. Note that the same approach could have been applied to all of the other types of models tested. The mean of these absolute errors (MAE) was then used to evaluate the overall performance of each model, as this metric is robust to outliers:

$$M A E = \frac{1}{n} {\sum }_{i = 1}^{n} \vert {y}_{i} - {\hat{y}}_{i} \vert$$

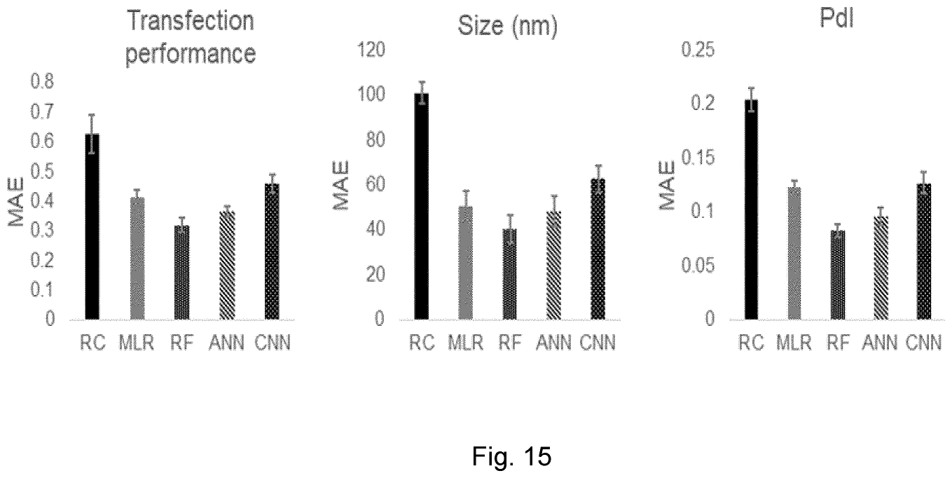

Given the small number of data points available in the training data, the neural network approaches were not expected to outcompete other modelling strategies, as increasing model complexity generally requires many examples to avoid performances losses due to overfitting. However, the inventors showed that even with the small proof-of-concept training dataset, all models could out-perform the RC baseline, and that both the RF and ANN models outperform the MLR model demonstrating the importance of non-linear feature associations (see FIG. 15 which shows the 10-fold cross validation overall model performance comparison with bar charts showing the MAE±standard error). The MAEs across the 10-folds for all the models were:
for normalised transfection: RC=0.63, MLR=0.41, RF=0.32, ANN=0.37, CNN=0.46;
for size (nm): RC=100.96, MLR=50.48, RF=40.51, ANN=48.54, CNN=62.97;
for PdI: RC=0.20, MLR=0.12, RF=0.08, ANN=0.10, CNN=0.13.

Thus, the RF and ANN models outperformed the baseline (random choice) model for all predicted characteristics. The CNN did not, but this may be related to the relatively small size of the training data set.

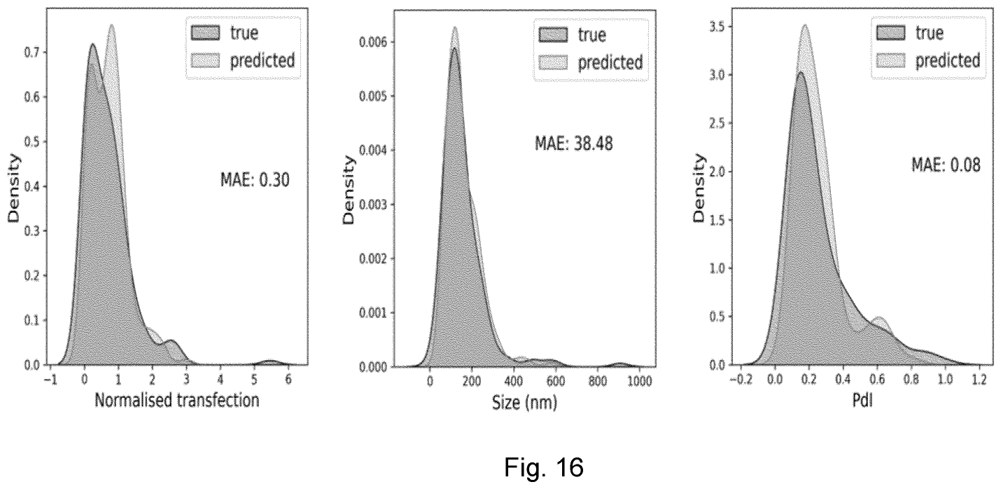

Results of LOOCV for the best model (RF) are visualised by displaying the KDE plots of the true target value distributions overlaying the predicted distributions (FIG. 16). The same bandwidth was applied to the true and predicted values, defined by initially applying Scott's rule to the true values. This shows a strong agreement (and low LOOCV MAE) between data and predictions for all of the predicted features.

### Example 6—Real-World Validation

In this example, the performance of the models trained in Example 4 was evaluated in a real-world scenario. Indeed, although a model's performance can be estimated using in silico methods like k-fold cross-validation or bootstrapping, this does not always provide an accurate assessment of how well it will generalise to unseen data; especially if that data contains examples that are dissimilar to the those found in the training set. It is also important that any computational model designed to predict nanocarrier performance is practically useful, being able to help guide the design of novel nanocarrier formulations for enhanced performance in real-world scenarios.

For these reasons, the inventors conducted a proof-of-concept experiment for real-world validation, where the best performing model (the RF) was used to help guide the design of novel nanocarriers which were subsequently synthesised and tested in the laboratory. They used two separate approaches to select new nanocarriers to synthesise: (i) a purely computational approach, where 943495 random nanocarrier formulations were generated; and (ii) an ML-guided approach, where 140 formulations (20 different dendrimers evaluated across 7 different N/P ratios) were initially conceived by human experts followed by ML-based sorting to select the theoretical best of these designs. In both cases the RF model was used to predict the in vitro transfection performance, size (nm), and PdI of these nanocarrier designs. The random nanocarriers were obtained by randomly sampling from possible naturally occurring amino acids with the following constraints: (i) each nanocarrier is simulated with NP from 0.16 to 16 in 7 increments (0.16, 0.6, 2, 4, 8, 12, 16), based on values experimentally tested by the inventors (i.e. 134795 different dendrimers sequences at 7 different NP ratios were simulated); (ii) maximum 55 residues in the dendrimer; (iii) at most 60% hydrophobic residues outside of the core sequence (hydrophobic residues are considered to be ay residue selected from ‘F’,‘I’,‘L’,‘M’,‘V’,‘W’,‘Y’), reflecting an assumption that very hydrophobic peptides are more difficult to manufacture; (iv) at least one cationic residue in the final generation; (v) at most one cysteine in the peptide, in the core only (no cysteine outside of core), reflecting an assumption that this may create manufacturing issues; (vi) no more than 2 W (Tryptophan) or Y (Tyrosine) adjacent to each other in the sequence, reflecting an assumption that this complicates manufacturing.

Formulations from the first approach (i) were ranked by their predicted transfection performance and the top 3 performers (highest predicted transfection efficiency) selected for real-world synthesis (R1, R2, and R3). In the ML guided case (ii), designs were also ranked by their predicted transfection performance, but 4 were selected (E1, E2, E3, and E4) amongst the top 10. Although in the present case only the predicted transfection performance was used to select candidates for testing, it is also possible to consider the other structural features when selecting candidates, for example to inform the selection in terms of which formulations are likely to result in monodisperse nanocarriers, nanocarriers with relatively narrow size distributions and nanocarriers with sizes suitable for an intended use. This may be particularly important when considering a larger number of nanocarriers for testing as applying additional criteria beyond transfection performance may reduce the risk of producing large numbers of nanocarriers that are not fit for purpose. Designs were then synthesised at both their predicted best N/P ratios along with two additional N/P ratios to expand the dataset for statistical analysis (in case some formulations did not form stable particles). Thus, each of these best formulations was made at a NP of 0.6, 4 and 8. In total, 21 unique nanocarrier formulations were synthesised for real-world validation. 11 out of 21 formulations (5/9 random generated designs, 6/12 human designed, indicating that a fully automated approach may have similar performance as an expert guided approach) formed good particles, according to subsequent DLS measurements (PdI<0.25). Selection based on PdI (i.e. selecting for low predicted PdI in addition to high predicted transfection efficiency) may have further improved the proportion of selected candidates that formed good particles (measured PdI<0.25). Neverthelesss, given that the NP ratio of the nanoparticles with highest predicted transfection efficiency tended to be high, and that most particles have higher PdI at higher NP ratios (e.g. in the training data, 100% of NP 0.6 had PdI<=0.25, 65% of NP 4 had PdI<=0.25
and 44% of NP 8 had PdI<=0.25), a>50% success rate in the selected candidates is a very good result. The tendency of the model to predict higher transfection efficiency for nanoparticles may be at least in part because the model has learned that nanoparticles with high NP and low PDI, although rare, tend to perform better than equivalent low NP nanoparticles. These were taken forward for in vitro transfection performance testing.

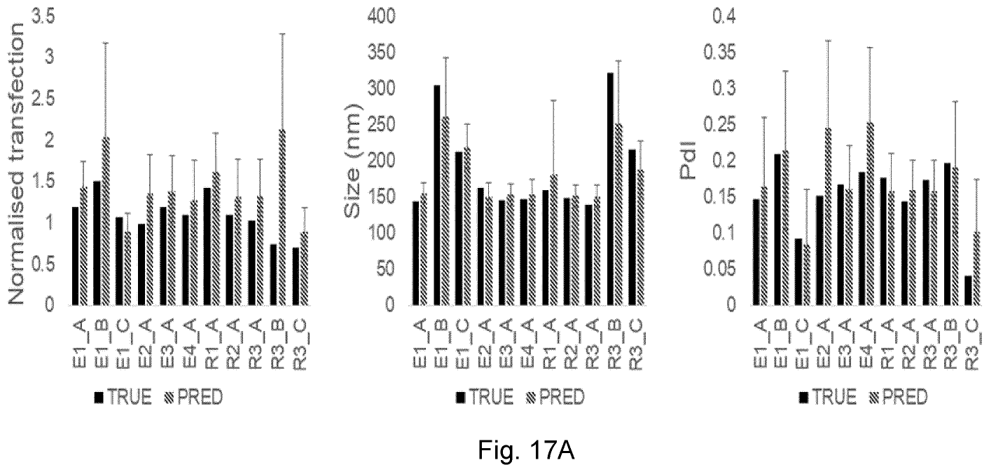

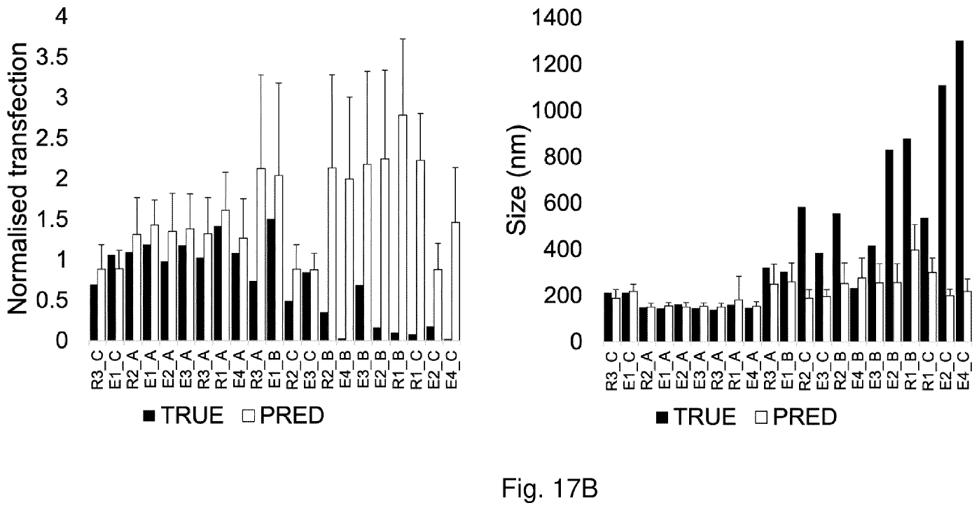

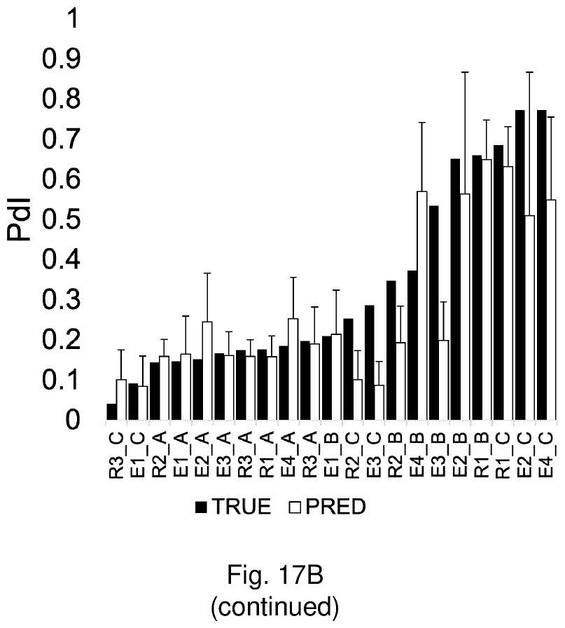

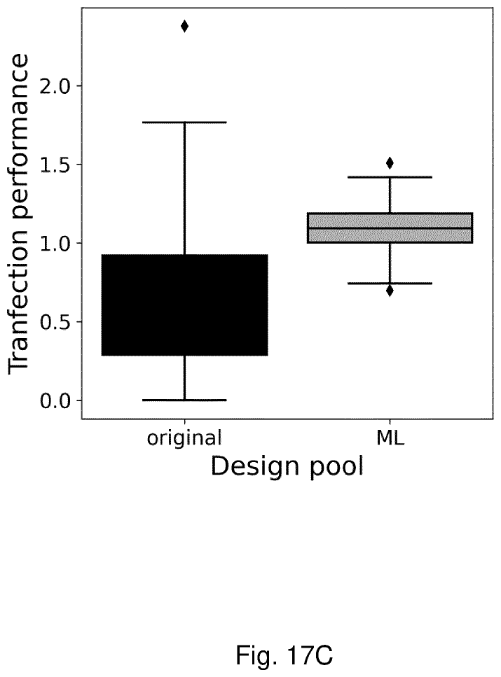

The experimentally observed values for the structural and functional properties of these nanocarriers are compared to their predicted values in FIG. 17A (which is a bar chart showing the transfection performance, size and PDI observed vs predicted using a RF model, the error bars on predictions are the standard deviations across predictions from the trees in the forest). The model demonstrated good generalisability in real-world validation, with an overall MAE of 0.36 for normalised transfection performance, 19.93 for size, and 0.03 for PdI. Moreover, for poorer individual predictions, standard deviation across estimators in the RF was large, showing low model confidence (i.e. the model demonstrates some capacity to “know when it doesn't know”). FIG. 17B shows the same data as FIG. 17B but including nanocarriers with predicted pdi>0.25 (which were excluded from FIG. 17A as FIG. 17A focussed on “good particles”), and with the nanocarriers arranged by increasing measured PDI. This shows that the method was able to predict high PDIs, indicating that the method can be used to exclude candidates that are likely to form particles that do not have suitable PDIs (“bad particles”). In other words, a model prediction of a PDI that is too high for further use is informative. This also shows that the predictions of transfection performance and size are less reliable for the nanocarriers that have high PDIs, likely because these formulations do not result in well-formed mono-disperse particles. This motivates an approach whereby the model can reliably be used to predict nanocarriers that should be excluded based on poor PDI (regardless of predicted size and/or transfection efficiency). For example, an approach comprising ranking the nanocarriers based on predicted transfection efficiency and excluding nanocarriers with predicted PDI above a threshold (e.g. 0.25) may be particularly advantageous. Finally, FIG. 17C shows box plots of measured transfection performance for the training data set described in Examples 1 and 2, and the nanocarriers selected using machine learning as described in this example (all filtered to exclude nanocarriers with PDI>=0.25). This shows that the machine learning approach enables the selection of nanocarriers with improved transfection efficiency (Mann-Whitney U test showed the difference between the two sets is highly significant p<0.001), underlining the practical use of the method.

### Example 7—PDI Classification

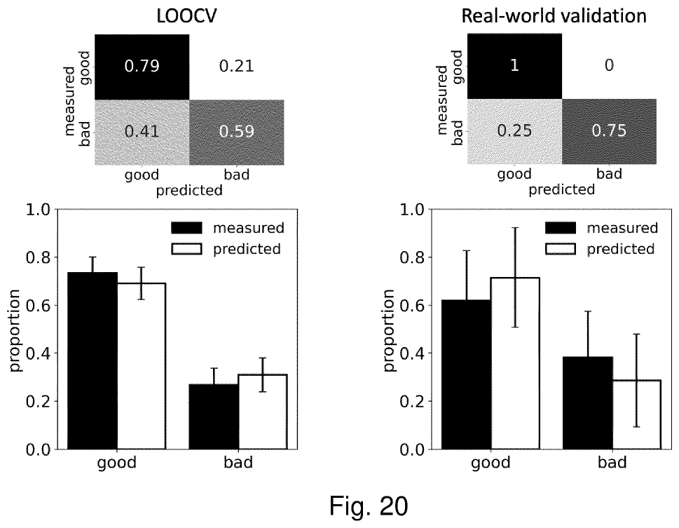

The above data shows that the PDI prediction model is able to confidently identify “good quality” nanocarriers from those that would not typically be deemed acceptable for therapeutic delivery (PDI≥0.3; Danaei et al., 2018). To further investigate this property, the inventors determined model performance relative to a threshold (in this case PDI<0.3). In order to do this, a regression model can be treated as a classifier, with predicted values falling below the threshold treated as ‘positive’, and those falling at or above the threshold treated as ‘negative’. To evaluate classification performance for PDI thresholds the inventors used F₁ score, which calculates the harmonic mean of precision and recall as:

$${F}_{1} = \frac{T P}{T P + 1 / 2 ( F P + F N )}$$

where TP is the number of true positives, FP is the number of false positives, and FN is the number of false negatives. In this case the F₁ score obtained from LOOCV on the training set (Example 2) was 0.82 with a PDI threshold of <0.3 defining a good quality sample (FIG. 20A). Importantly, for the real-world validation set, the data on FIG. 20B shows that the PDI prediction model was able to sort good quality samples from those that would not typically be deemed acceptable for therapeutic delivery (PDI≥0.3) with very high accuracy (F1=0.93).

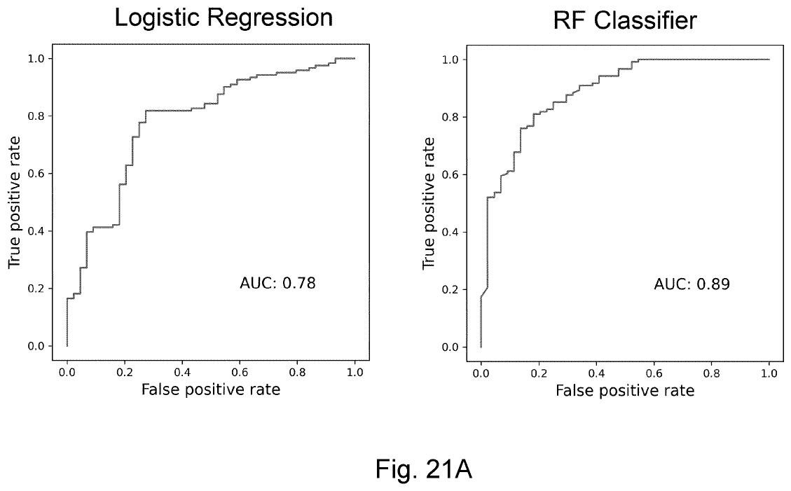

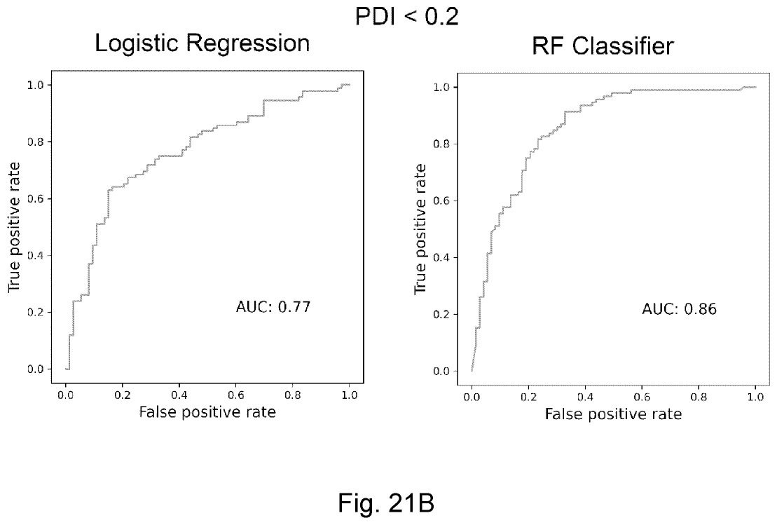

One could also classify the PDI directly by simply pre-labelling examples with PDIs above or below a particular desired threshold as 0 or 1 to define “good” or “poor” quality particles and using a slightly different cost function for ML modelling (e.g. binary cross-entropy). This is demonstrated below. In particular, PDI classification models were trained on the same dataset as the original PDI regression model and similarly evaluated using either LOOCV or on the real world validation set (see Examples 4 and 6 above). Features were the same and were extracted in the same way as in Example 3. In this case the threshold of <0.3 PDI was chosen to denote good particles, and these were assigned 1 (“positive”) with anything at or above this threshold of 0.3 labelled as 0 (“negative”). A RF classifier and a logistic regression model were trained. The random forest classifier had the same number of trees/estimators as the random forest regressor of Example 4 (512) and the same hyperparameters as described in Example 4. The comparative logistic regression model had default parameters with an L2 penalty for regularisation (ridge regression). Direct classification demonstrated similar performance to indirect post-regression classification for the random forest classifier. The F₁ score obtained from LOOCV on the training set was 0.9, and for the real world test set the score was 0.87. The random forest also outperformed the logistic regression model which obtained an F₁ score of 0.82 for LOOCV, and 0.78 on the real world test set. FIG. 21A shows ROC (receiver operating characteristic) curves for both logistic regression and the random forest classifier, also demonstrating better performance using the random forest classifier. The same work process was repeated for the threshold of <0.2 PDI, and the results of this are shown on FIG. 21B. This also shows a very good classification accuracy.

Thus, this data demonstrates that both prediction of a continuous PDI value (regression) and prediction of a class of PDI values (classification) are suitable for the purpose of identifying “good” vs “bad” nanocarriers with high accuracy. The regression approach advantageously does not lead to information loss regarding the specific PDI, and has the additional benefit of enabling adjustment of the desired quality threshold post-training (as classification is applied on the output of the model, where the model is unchanged regardless of the threshold applied for subsequent classification).

### Example 8—Zeta Prediction

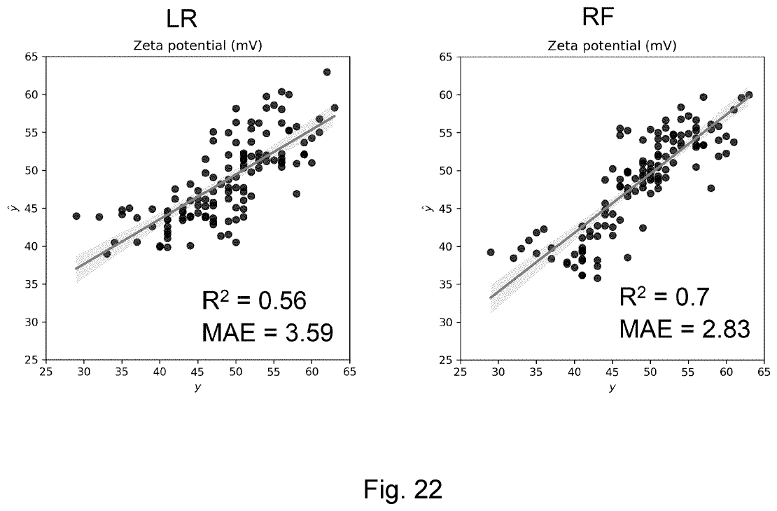

In this example, the inventors built a model to predict the zeta potential of microcarriers. There were slightly fewer examples (n=124) for predicting Zeta than other physicochemical attributes of the nanocarriers demonstrated above (size and PDI), but all examples were drawn from the same pool as used for the other structural attribute prediction models (described in Examples 1 and 2). Prediction models were evaluated using LOOCV. Features were the same and extracted in the same way as in Example 3. A linear model and RF model were trained. These were the same as those described in Example 4 and used the same hyperparameters. Both modelling approaches showed good performance evaluated by LOOCV, although the random forest clearly outperformed linear regression (R²=0.56 vs 0.7). FIG. 22 shows scatter plots comparing the two approaches with associated statistics. This data demonstrates that the Zeta potential can be predicted with reasonable accuracy using the methods described herein, and that non-linear models are likely to be preferable for this task.

### Example 9—Feature Importance Permutation

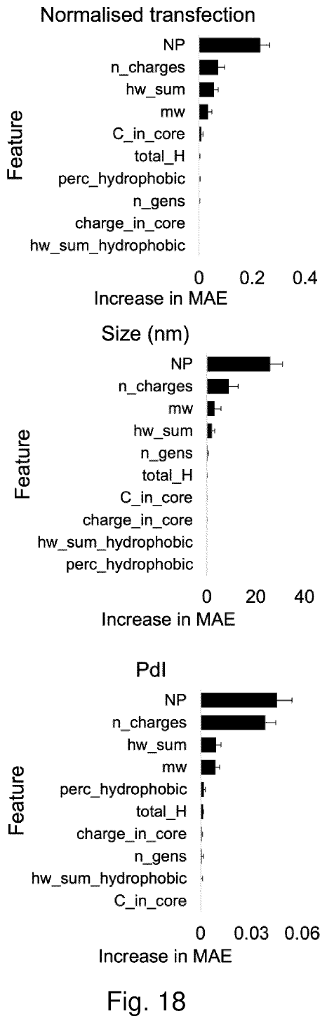

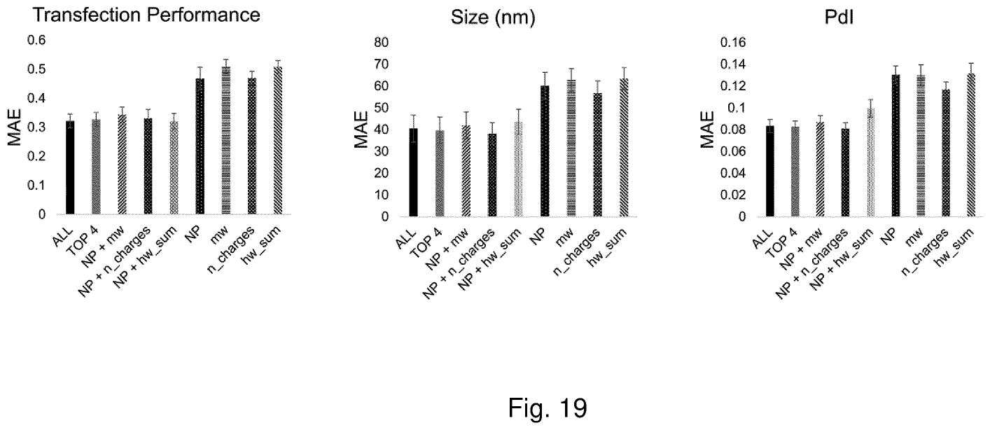

In this example, the inventors investigated which input features (predictive features) are of most importance to the output (predicted features) of the machine learning models obtained. Indeed, another way to guide nanocarrier design is to use computational models to highlight features that may be fundamental for achieving experimental aims. This information can then be used by human experts to inform nanocarrier design decisions. MLR and RF models have their own canonical ways to rank the importance of contributory features by either assessing the magnitudes of slope coefficients (MLR) or plotting the mean decrease in impurity (Gini importance) across trees (RF), respectively. These are powerful techniques but are not generalisable across ML model types. Furthermore, MLR coefficients can only show linear contributions, and Gini importance has been shown to be biased, especially toward categorical variables (Loecher, 2022). Both feature ranking techniques also tend to spread importance across collinear features, which can hide the true individual effect of inputs on a particular response variable of interest. Permutation importance is a generalisable technique where predictions are made on a test set after a feature of interest has been shuffled so that its contribution is lost (Nicodemus et al. 2010). This was performed iteratively for each input feature, the effect on the model performance was assessed after each iteration, and the features were ranked by magnitude of effect. The features considered to be most important would be those which cause the largest drops in accuracy or increases in error after shuffling. This approach can also be used to assess the contributions of different combinations of features by shuffling multiple features at once. FIG. 18 shows the results of performing permutation feature importance testing with the RF model for each of the 10 input features (for each target, the increase in MAE is shown as the feature values are randomly shuffled). The experiment is repeated using multiple sets of hold-out data by employing 10-fold cross validation. FIG. 19 shows the results of 10-fold cross validation of models trained using a subset of the predictive features as indicated (average MAE are shown with standard deviations).

The data on FIG. 18 shows that the NP feature is most predictive of all parameters predicted, followed by the number of charges in the dendrimer, the molecular weight of the dendrimer, and the sum of the Hopp-Woods hydrophilicity scores from each residue in the dendrimer. Note that the training data was limited in terms of the diversity of the dendrimer sequences used, which may have reduced the importance of at least peptide-specific features. Further, the training data only contained nanoparticles with a lipid-payload ratio of 10, preventing the model from learning the importance of the lipid-ratio in the transfection efficiency. Based at least in part on the finding that the NP (peptide-payload) ratio was found to be a particularly important predictive feature, it is likely that the lipid-payload ratio will also be highly predictive of transfection efficiency. The data on FIG. 19 shows that a model using only the top 4 predictive features (in this case NP, number of charges in the dendrimer, the molecular weight of the dendrimer, and the sum of the Hopp-Woods hydrophilicity scores from each residue in the dendrimer) had a similar performance as a model using all features, as did models including NP and any one of the other 3 features of the top 4. Models using any one of the top 4 predictive features alone had higher errors but still provided useful predictions.

### Example 10—Predicting Transfection Efficiency with Additional Data and Features

In this example, the inventors demonstrate the training and evaluation of models that have additional advantages compared to some of the models described above. In particular, they trained a neural network model using a larger training dataset including more nanocarriers as described in Example 1, as well as lipid-only nanocarriers (lipoplexes). Additional predictive features were included in the model, including features related to the lipid to payload ratio.

Data preparation: The new dataset initially contained 314 examples (1.9× the size of the original dataset). This data comprises nanocarriers as described in Example 2, except that a variety of lipid ratios were represented. In particular, the nanocarriers in Example 2 all had a lipid ratio of 10, whereas the nanocarriers used here included all the nanocarriers in Example 2 and versions thereof with a lipid ratio selected from 5, 7.5, 10 and 12.5.

A PDI threshold of <0.3 was then applied to ensure that only high-quality particles were included in the final training set. This left 238 (76%) of total nanocarriers for modelling. Of these, approximately 10% were held out randomly as an independent test set, providing 214 examples for training, and 24 for subsequent evaluation. This filtering was performed because training a model to predict transfection efficiency using examples of varying particle quality, including nanocarriers deemed to be outside of a desired PDI range, could cause the model to waste resources learning an unnecessary association between particle quality and function. This can be mitigated by applying a quality threshold to the outputs of the trained model, as demonstrated in example 6. However, if the threshold is known, it may be more practical to train the functional model (e.g. for transfection performance) only on examples that have high quality (e.g. low PDI) to begin with. This way the model only learns associations between features of high-quality particles and the functional metric. In practice for unknown nanocarriers a first model may be used to predict or classify PDI as described above (or PDI may be measured), and only if the unknown nanocarrier is predicted or otherwise shown to have acceptable PDI then functional properties like transfection efficiency are predicted using a model that has been trained using data for nanocarriers that have acceptable PDI.

The mean normalised transfection efficiency was taken as the ground truth target. To ensure that the distribution of the random hold-out set was similar in composition to the training set, a standard t-test and nonparametric Mann-Whitney U test were performed. Neither found a significant difference between the target distributions of the training set and independent test set (p-values 0.25, and 0.36 respectively).

Features: Features were extracted, as described in Example 3. However, several new features were also generated increasing the total number of features to 23 (Table 2), including a feature to account for lipid to payload ratio (L ratio). Three lipid-only (no dendrimer) examples (lipoplexes) were also included in the training set at L ratios 5, 7.5, 10, and 12.5. No lipoplexes were included in the testing set. The models in the above examples did not account for different lipid ratios which could affect nanocarrier performance, and did not include lipid-only (no dendrimer) examples in the training set (e.g. simple lipoplexes), which may be useful for providing a baseline for the model. For training the present model, four lipoplexes were included made of DOTMA+DOPE 1:1+payload. These are the same lipids as those used in the other nanocarriers in this example and Example 2, just lacking dendrimer. The 4 lipoplexes differed only by the amount of lipid used relative to mRNA payload (i.e. lipid ratio of 5, 7.5, 10 or 12.5).

All features were min-max scaled between 0 and 1 before modelling, but for the lipoplex examples, dendrimer-derived features were simply assigned the value-1 to denote the lack of that dendrimer feature.

TABLE 2
Description of input features for machine learning.

| Feature name | Description |
|---|---|
| NP | The ratio of positively charged amine nitrogen (N) in the peptide |
| | dendrimer (or peptide/protein component) to phosphate in the |
| | nucleic acid payload. |
| n_gens | The number of generations comprising the branched dendrimer. |
| mw | The molecular weight of the dendrimer |
| n_charges | The number of positively charged sidechains in the dendrimer |
| | peptide sequence. |
| total_H | The total number of histidine residues in the dendrimer peptide |
| | sequence. |
| hw_sum | The sum of the Hopp-Woods hydrophilicity scores from each |
| | residue in the dendrimer. |
| hw_sum_hydrophobic | The sum of the Hopp-Woods hydrophilicity scores from each |
| | hydrophobic residue in the dendrimer. |
| perc_hydrophobic | The percentage of residues comprising the dendrimer sequence |
| | that are classed as hydrophobic. |
| C_in_core | Whether a cysteine residue resides in the dendrimer core |
| | sequence. |
| charge_in_core | Where a positively charged side chain is present in the dendrimer |
| | core sequence. |
| L Ratio | The w:w ratio of lipid components to the nucleic acid payload. |
| A205 | Computational estimation of absorbance of the dendrimer |
| | component at 205 nm, based on Anthis and Clore (2013). |
| A280 | Computational estimation of absorbance of the dendrimer |
| | component at 280 nm, based on Gill and von Hippel (1989). |
| Net charge at pH 7.4 | The net charge Z of the dendrimer component at pH 7.4 estimated |
| | from the amino acid composition using: |
| | $Z = {\sum }_{i} {N}_{i} \frac{1 {0}^{pKai}}{1 {0}^{pH} + 1 {0}^{pKai}} - {\sum }_{j} {N}_{j} \frac{1 {0}^{pH}}{1 {0}^{pH} + 1 {0}^{pKaj}}$ |
| | where Ni are the total quantities of Arginine, Lysine, and Histidine, |
| | and the pKai pKa of their side chains and N-termini; and Nj are the |
| | total quantities of Aspartic Acid, Glutamic Acid, Cysteine, Tyrosine, |
| | and pKaj the pKa of their side chains and C-termini. |
| Net charge at pH 6.5 | As above, but for pH 6.5. |
| Net charge at pH 5.5 | As above, but for pH 5.5. |
| Net charge at pH 4.5 | As above, but for pH 4.5. |
| pl | The isoelectric point of the dendrimer component, which is the pH |
| | at which the net charge is closest to 0. Estimated computationally |
| | by incrementing the pH by 0.01 between 0 and 14 using the above |
| | formula. |
| pl g3 | As above, but only for amino acids in the third generation of the |
| | dendrimer. If no third generation is present, the model receives the |
| | value −1 after feature scaling, indicating a missing value. |
| pl g2 | As above, but only for amino acids in the second generation of the |
| | dendrimer. If no second generation is present, the model receives |
| | the value −1 after feature scaling, indicating a missing value. |
| pl g1 | As above, but only for amino acids in the first generation of the |
| | dendrimer. |
| pl core | As above, but only for amino acids comprising the dendrimer core |
| | sequence. |
| pl fg | As above, but only for amino acids in the final, outermost, |
| | generation of the dendrimer. |

Note
that L ratio was used as the only lipid related feature because all training data available was usingthe same lipids, with varying L ratio.
This was therefore the only lipid related property for which training data was available which could inform the model.
Additional and/or other lipid related properties as described above could be used if training data is available comprising nanocarriers that have different values for these properties.

A205 and A280 are commonly used to quantify the amount of protein or peptide in a sample. However, as different amino acids have different absorbances, it can also give you a unique number/fingerprint associated with the sequence composition. A205 considers specific absorbance for ‘W’, ‘F’, ‘Y’, ‘H’, ‘M’, ‘R’, ‘C’, ‘N’, and ‘Q’ sidechains, along with a standard absorbance for the amino acid backbones (so can also consider sequence length, even when the above amino acids are not present). This produces values that are relatively specific to dendrimers. A280 is only representative of the sidechains of W, Y and C, and is therefore less informative in general (and less specific to each dendrimer).

Note that the pl fg value will be the same as pl g1 for a dendrimer with one layer, the same as pl g2 for a dendrimer with 2 layers, and the same as pl g3 for a dendrimer with 3 layers. However, considering the final layer in general (regardless of dendrimer structure) advantageously enables to compare the outer layer/generation of dendrimers with different structures, has the flexibility to provide information for a dendrimer of any structure, and reflects an important property of dendrimers of any structure.

Modelling approach: The inventors focussed on neural networks in this example. The benefits of using a tree-based approach, as demonstrated in the examples above, were better overall performance on the limited dataset (tree-based models were found to slightly outperform the other strategies in terms of lower MAE), and the ability to estimate model confidence for unseen examples by using aggregate statistics from individual trees in the forest. However, tree-based models, such as random forests, cannot extrapolate, meaning they cannot predict beyond the minimum and maximum ground truth values provided in a training set. This is not an issue for bounded measurements such as PDI, which can only take a value between 0 and 1; or where, beyond a particular threshold, any larger or smaller does not matter from a practical standpoint (e.g. size). It is also not a problem for classification problems where things are simply categorised into finite bins. However, if one intends to discover new nanocarrier formulations that are in silico predicted to outperform previously tested formulations, it is likely more practical to have a model that can extrapolate beyond the maximum previously obtained. Neural networks are able to do this, but typically require larger amounts of training data than tree-based approaches to obtain the same performance. This was mitigated by i) using a larger training set to obtain a more accurate model, and ii) using a Monte Carlo Dropout approach (Gal and Ghahramani, 2016) in order to obtain a measure of model confidence. A new random forest model was also trained on the new dataset to compare performance.

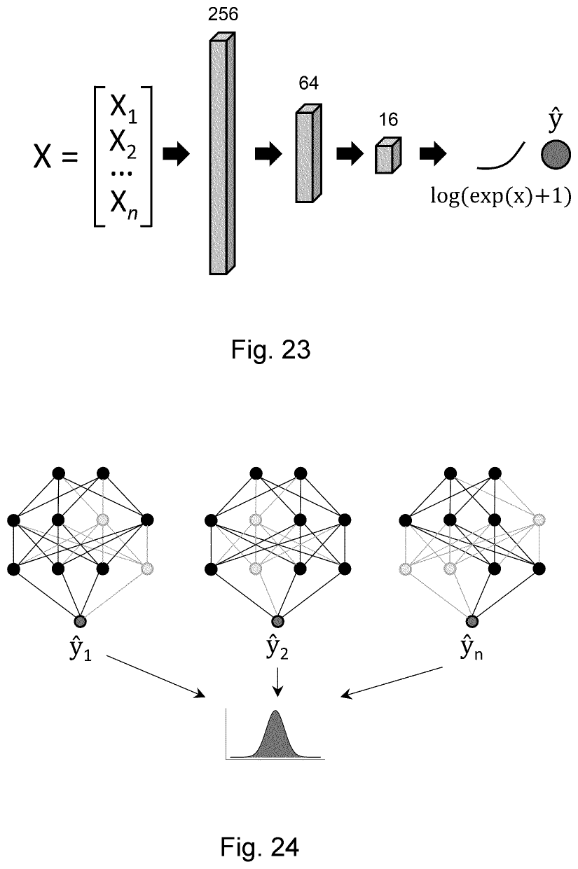

Grid-search and model training: To discover the best neural network architecture for the new features and training set, a grid search was performed using 5-fold cross validation. Given the size of the dataset, the network consisted of 3 hidden layers, each followed by ReLU activation functions, with the grid searching for the best architecture using either 16, 32, 64, 128, or 256 neurons in each layer. The loss function, optimisation function, and learning rate used were the same as in Example 4 for the original ANN. Dropout was also enabled after each hidden layer with a 50% dropout rate. Dropout is a technique where connections in the network are randomly disabled during training forcing the model to adapt, and not to become reliant on a few strong pathways through the network. This provides regularisation and prevents overfitting using a form of ensembling. Many more neurons could be used, and fewer or more layers could be used with a larger training set and/or if one employed data augmentation. The best architecture in this particular example was a network comprising 256 neurons in the first layer, 64 neurons in the second layer, and 16 neurons in the third layer (FIG. 23). The ideal number of epochs was determined using 10-fold cross validation on the training set and the number of epochs with the mean lowest loss used for final training. In this case the final model was trained for 881 epochs.

Estimating model confidence using Monte Carlo dropout: Typically dropout is disabled during inference, so that the fully connected trained network can be used to make predictions. However, this only provides a single predicted value for each new example. In contrast a random forest, for example, provides a predicted value that is the aggregate mean of all the decision trees comprising the forest. If one also obtains the variance across trees, one can estimate the confidence in an individual prediction made by the model. Monte Carlo Dropout provides a similar way to obtain confidence in a prediction for a neural network, by enabling dropout during the inference phase (Gal and Ghahramani, 2016). In this case, connections are randomly disabled within the trained network and prediction made for an example nanocarrier. This process is repeated n times, each time disabling a different set of connections (FIG. 24). The aggregate of predictions is then taken as the final prediction for the example, and the variance across the Monte Carlo iterations used to estimate confidence.

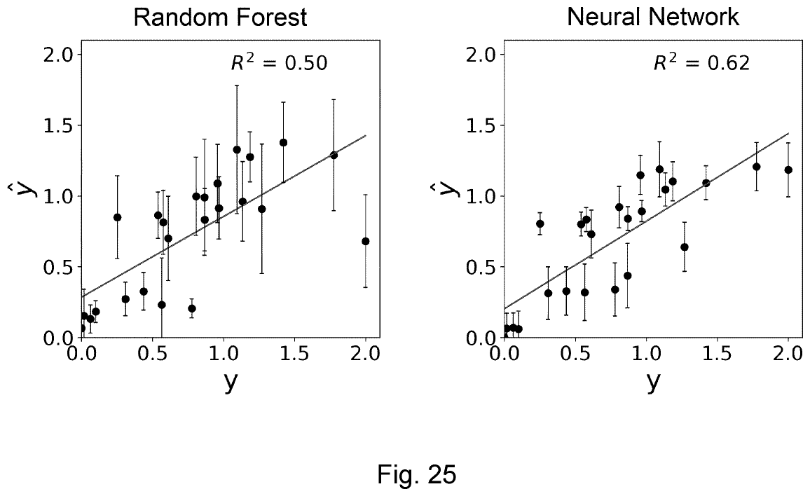

Results on the independent test set: FIG. 25 shows the performance of the ANN using Monte Carlo Dropout on the independent held-out test set, compared with a Random Forest model trained and tested on the same dataset, also using the augmented set of features described in Table 2. With more features and more examples available for training, it is clear that the neural network is now outperforming the Random Forest model.

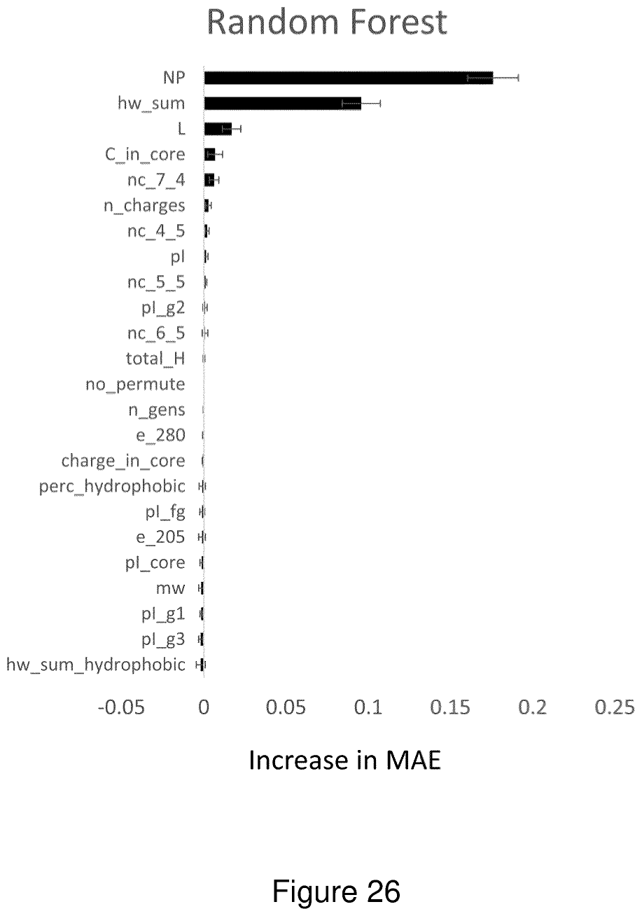

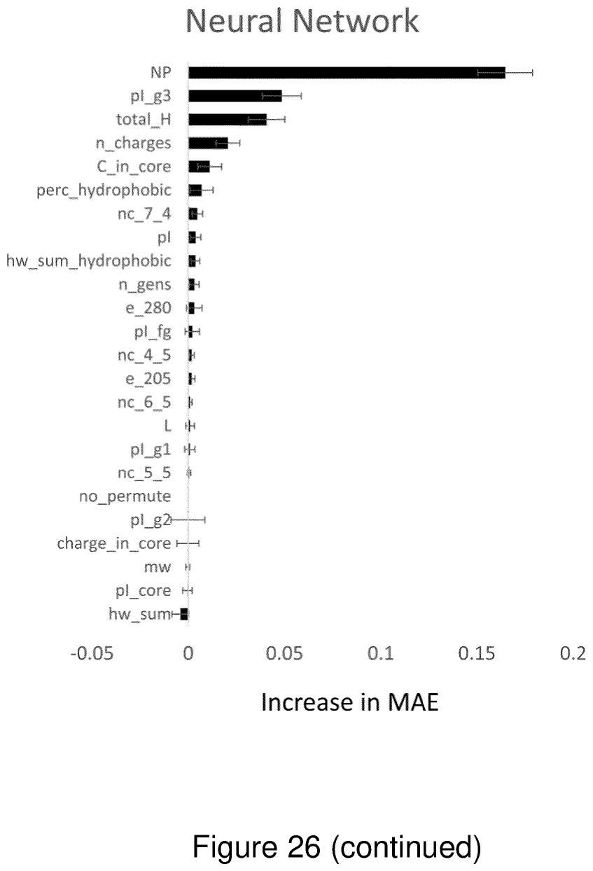

Feature importance analysis: FIG. 26 shows the results of a permutation importance analysis from either the random forest or neural network model. The analysis was performed as described in Example 9. Each bar shows the increase in error (MAE) as a respective feature is randomly shuffled prior to validation. Error bars show standard error.

As above, the data on FIG. 26 shows that the NP feature is most predictive of all parameters predicted, for both modelling approaches. For the Random Forest model the top 3 predictive features further included the lipid ratio (L) and the sum of the Hopp-Woods hydrophilicity scores from each residue in the dendrimer (hw_sum). Thus, two of the top three features for this model are also in the top predictive features from Example 9. As mentioned above, the data used in Example 9 only contained nanoparticles with a lipid-payload ratio of 10, preventing the model from learning the importance of the lipid-ratio in the transfection efficiency. Thus, the data for this model is broadly in agreement with the findings in Example 9. It further indicates that a random forest model including only the NP, L and hw_sum features should perform similarly as the full model. For the neural network features the importance was more spread out between the top 5-6 features, although NP was still clearly the most important feature. The isoelectric point for the third generation of the dendrimer and the total number of histidines were both relatively important predictors. It is possible that the pl_g3 feature was informative at least to some extent because it provided information about the size of the dendrimer (since dendrimers with fewer than 3 generations were assigned a default value). Thus, this feature may be providing information that at least partially overlaps with the molecular weight of dendrimer feature that was found to be informative in Example 9. These were followed by the number of charges (which was also an important predictor of the model in Example 9), the number of Cysteine residues in the core of the dendrimer (which is a top 4 feature in the Random forest model of the present example), and the percentage of hydrophobic residues (which may enable this model to capture similar information to the hw_sum feature in the random forest model).

Thus, the data shows that models that include the NP feature and one or more features that capture hydrophobicity/hydrophilicity properties of the peptide component (such as e.g. number of charges in the peptide component, the sum of the Hopp-Woods hydrophilicity scores from each residue in the peptide component, percentage hydrophobic residues, total number of histidines) are likely to provide informative predictions. Further, the data shows that the lipid ratio feature may also be informative when the training data comprises nanocarriers with different L. When the nanocarrier for which the prediction is being made has the same L as a substantial proportion of the nanocarriers in the training data, the data in Example 9 shows that this property is not necessary to obtain good predictions. Finally, the data shows that a feature that captures information about the size of the peptide component may also be informative.

### Example 11—Conclusions

In the examples above, the inventors have shown that it is possible to predict nanocarrier attributes using machine learning models, and that predictive performance can generalise to new unseen formulations and help guide the design of more effective nanocarrier formulations.

Preliminary data shows that the nanocarriers' in vitro performance in HeLa translates to specific other cell types and specific in vivo situations (based on experiments in mice), but strong cell-type specific behaviours were also observed. Thus, the models described herein can be used to predict transfection performance in a robust manner, and cell type/tissue specific models can be developed using the approaches described herein.

The inventors believe that the approach outlined in these examples can be expanded to predict other nanocarrier attributes that can be quantified numerically or categorised (using either regression models or classification models, depending on whether a class or continuous prediction is desired). In particular, the inventors believe that the approach can be used to predict cell-specific payload delivery (e.g. predicting transfection efficiency in multiple cell types, to determine whether some nanocarriers show different behaviours in different cell types such that payload delivery that is specific to a cell type, e.g. monocytes/macrophages, can be obtained), tissue specific payload delivery (e.g. predicting transfection efficiency in a tissue specific manner), in vitro and in vivo cytotoxicity (e.g. using live/dead staining data in vitro as training data), in vitro and in vivo immunogenicity, nanocarrier temperature dependent structural stability and integrity (e.g. using training data from freeze-thaw experiments to assess how stability is affected), nanocarrier pH dependent structural stability, integrity, performance, and behaviour (e.g. using training data from experiments in which the nanocarriers are incubated in low pH such as eg. pH≈5—this may be useful to predict the state of the nanoparticles in the lysosome compared to a more neutral pH environment such as blood/cytoplasm), and nanocarrier concentration dependent structural stability, integrity, performance, and behaviour (e.g. using training data comprising DLS and transfection performance at various concentrations).

Additionally, even though in the present examples separate models were trained to predict each of the properties of interest, the inventors believe that multi-output models can be produced that could predict multiple nanocarrier properties simultaneously from the shared input features. These may have slightly better prediction performances particularly in cases where there are strong associations between the plurality of characteristics predicted (as the model can then learn from one characteristic to predict another). Multi-output models may also suffer less from overfitting.

### REFERENCES

A number of publications are cited above in order to more fully describe and disclose the invention and the state of the art to which the invention pertains. Full citations for these references are provided below.
Anthis, N. J., and Clore, G. M. (2013) Sequence-specific determination of protein and peptide concentrations by absorbance at 205 nm. Protein Science 22, 6.
Benizri et al. Bioconjugated Oligonucleotides: Recent Developments and Therapeutic Applications. Bioconjug Chem. 30 (2): 366-383. (2019).
Bonnet et al. Systemic Delivery of DNA or siRNA Mediated by Linear Polyethylenimine (L-PEI) Does Not Induce an Inflammatory Response. Pharmaceutical Res. 25, 2972 (2008).
Braum. Non-viral Vector for Muscle-Mediated Gene Therapy; Chapter 9 Muscle Gene Therapy, Springer Nature Switzerland AG D. Duan, J. R. Mendell (eds.) 157-178 (2019).
Breiman, L., 2001. Random forests. Machine learning, 45 (1), pp. 5-32.
Danaei, M., et al. (2018). Impact of particle size and polydispersity index on the clinical applications of lipidic nanocarrier systems. Pharmaceutics 10, 57.
Gal, Y., and Ghahramani, Z. (2016). Dropout as a Bayesian Approximation: Representing Model Uncertainty in Deep Learning. 33rd International Conference on Machine Learning (ICML 2016).
Gill, S. C., and von Hippel, P. H. (1989). Calculation of protein extinction coefficients from amino acid sequence data. Analytical Biochemistry 182, 2.
Glorot, X. and Bengio, Y., 2010 March. Understanding the difficulty of training deep feedforward neural networks. In Proceedings of the thirteenth international conference on artificial intelligence and statistics (pp. 249-256). JMLR Workshop and Conference Proceedings.
Hopp, T. P. and Woods, K. R., 1981. Prediction of protein antigenic determinants from amino acid sequences. Proceedings of the National Academy of Sciences, 78 (6), pp. 3824-3828.
Huang et al. Delivery of Therapeutics Targeting the mRNA-Binding Protein HuR Using 3DNA Nanocarriers Suppresses Ovarian Tumor Growth. Cancer Research. 76 (6), 1549-1559 (2016).
Jasinski et al. The Effect of Size and Shape of RNA Nanoparticles on Biodistribution. Mol Ther. 26 (3), 784-792 (2018).
John et al. Human MicroRNA Targets; PLOS Biology, 11 (2), 1862-1879 (2004).
Kingma, D. P. and Ba, J., 2014. Adam: A method for stochastic optimization. arXiv preprint arXiv: 1412.6980.
Kwok et al. Comparative structural and functional studies of nanoparticle formulations for DNA and siRNA delivery; Nanomedicine: Nanotechnology, Biology and Medicine 7; 210-219 (2011).
Kwok et al. Peptide Dendrimer/Lipid Hybrid Systems Are Efficient DNA Transfection Reagents: Structure-Activity Relationships Highlight the Role of Charge Distribution Across Dendrimer Generations; ACS Nano 7; 5 4668-4682 (2013).
Kwok et al. Systematic Comparisons of Formulations of Linear Oligolysine Peptides with siRNA and Plasmid DNA; Chem Biol Drug Des 87:747-763 (2016).
Kwok et al. Developing small activating RNA as a therapeutic: current challenges and promises Therapeutic delivery 10 (3): 151-164 (2019)
Kyte, J. and Doolittle, R. F., 1982. A simple method for displaying the hydropathic character of a protein. Journal of molecular biology, 157 (1), pp. 105-132.
Hou et al., Lipid nanoparticles for mRNA delivery. Nature Reviews Materials volume 6, pages1078-1094 (2021).
Lim et al. Engineered Nanodelivery Systems to Improve DNA Vaccine Technologies; Pharmaceutics 12 (1) 30 (2020).
Loecher, M., 2022. Unbiased variable importance for random forests. Communications in Statistics-Theory and Methods, 51 (5), pp. 1413-1425.
Luo et al. Arginine functionalized peptide dendrimers as potential gene delivery vehicles. Biomaterials 33, 4917-4927 (2012).
Myers et al. Recombinant Dicer efficiently converts large dsRNAs into siRNAs suitable for gene silencing; Nature Biotechnology 21:324-328 (2003).
Nicodemus, K. K., Malley, J. D., Strobl, C. and Ziegler, A., 2010. The behaviour of random forest permutation-based variable importance measures under predictor correlation. BMC bioinformatics, 11 (1), pp. 1-13.
Philippidis. Fourth Boy Dies in Clinical Trial of Astellas' AT132. Human Gene Therapy. 32, 19-20 (2021).
Qiu et al. Developing Biodegradable Lipid Nanoparticles for Intracellular mRNA Delivery and Genome Editing. Acc. Chem. Res. 54 (21), 4001-4011 (2021).
Ren et al. Structural basis of DOTMA for its high intravenous transfection activity in mouse; Gene Therapy 7, 764-768 (2000).
Saher et al. Novel peptide-dendrimer/lipid/oligonucleotide ternary complexes for efficient cellular uptake and improved splice-switching activity; Eur J Pharmaceutics and Biopharmaceutics 132:29-40 (2018).
Saher et al. Sugar and Polymer Excipients Enhance Uptake and Splice-Switching Activity of Peptide-Dendrimer/Lipid/Oligonucleotide Formulations. Pharmaceutics. 11 (12): 666 (2019)
Scott, D. W., 2015. Multivariate density estimation: theory, practice, and visualization. John Wiley & Sons.
Sheridan et al. Gene therapy finds its niche. Nat Biotechnol 29 (2), 121-8 (2011).
Sloas et al. Engineered CAR-Macrophages as adoptive immunotherapies for solid tumors. Front. Immunol. 12:783305. (2021)
Tan, M. and Le, Q., 2021 July. Efficientnetv2: Smaller models and faster training. In International Conference on Machine Learning (pp. 10096-10106). PMLR.
Wang et al. Adeno-associated virus vector as a platform for gene therapy delivery. Nat Rev Drug Discov. 18 (5): 358-378 (2019).
Yang, F., Moss, L. G. and Phillips, G. N., 1996. The molecular structure of green fluorescent protein. Nature biotechnology, 14 (10), pp. 1246-1251.
For standard molecular biology techniques, see Sambrook, J., Russel, D. W. Molecular Cloning, A Laboratory Manual. 3 ed. 2001, Cold Spring Harbor, New York: Cold Spring Harbor Laboratory Press

1. A computer-implemented method of predicting one or more properties of a nanocarrier, wherein the nanocarrier is a non-viral cell delivery system, the method comprising:
Obtaining the value of one or more structural and/or functional properties of the nanocarrier; and
Predicting the values of one or more properties of the nanocarrier by providing the determined value(s) as input to a machine learning model that has been trained to take as input the values of one or more input structural and/or functional properties of a nanocarrier and produce as output the values of one or more output functional properties and optionally one or more output structural properties of a nanocarrier different from the input structural properties of the nanocarrier;
wherein structural properties of a nanocarrier characterise physico-chemical properties of the nanocarrier and are independent of the activity of a nanocarrier payload, and wherein the predicted one or more output functional properties of the nanocarrier comprise the transfection efficiency.

2. The method of any preceding claim, wherein the nanocarrier is a lipid-based nanoparticle, a peptide-containing nanoparticle, or a peptide containing lipid nanoparticle, optionally wherein the nanoparticle is a peptide dendrimer/lipid hybrid nanoparticle.

3. The method of any preceding claim, wherein the nanocarrier comprises a nucleic acid payload.

4. The method of any preceding claim, wherein the input properties comprise input structural properties and the output properties comprise output structural properties, and wherein the input structural properties of the nanocarrier comprise or consist of properties that are quantified in silico and/or wherein the output structural properties of the nanocarrier are properties that are experimentally determined.

5. The method of any preceding claim, wherein the determined structural properties of the nanocarrier are individually selected from global nanocarrier structural properties and component specific structural properties, optionally wherein component specific properties are individually selected from lipid-specific properties and peptide-specific properties.

6. The method of claim 5, wherein the determined global nanocarrier specific structural properties are selected from: protein to payload ratio, lipid to payload ratio, a size-related metric, a charge-related metric, optionally wherein the protein to payload ratio is the N/P ratio and/or wherein the charge-related metric is the zeta potential, and/or wherein the size-related metric is the hydrodynamic size or the polydispersity index and/or the lipid to payload ratio is the L ratio, and/or the hydrophobicity/hydrophilicity related metric is selected from.

7. The method of claim 5 or claim 6, wherein the component specific structural properties are selected from:
lipid-specific properties, optionally selected from: lipid identity, lipid type, ratio of different lipids or lipid types, length of lipid chains, lipid melting point, lipid saturation, molecular weight;
peptide-specific properties, optionally selected from molecular weight, charge, mass to charge ratio, scores indicative of the hydrophilicity and/or hydrophobicity of peptides or amino acids, extinction coefficient, isoelectric point, sequence length, presence of specific residues in the sequence, absence of specific residues in the sequence, number of specific residues in the sequence, proportion of specific residues in the sequence, number of branch points in a branched peptide, number of generations in a branched peptide, absorbance at a particular wavelength; and
learned features derived from local structural properties, wherein learned features are features identified by a trained machine learning model from a multidimensional input, wherein the local structural properties are structural properties of individual chemical entities within a component, optionally wherein the individual chemical entities are atoms, lipid chains or amino acids.

8. The method of any preceding claim, wherein the input structural properties of the nanocarrier comprise
any one or more of: the protein to payload ratio, the lipid to payload ratio, the number of positively charged sidechain(s) of the amino acid(s) in a peptide component or peptide payload of the nanocarrier, the number of negatively charged sidechains in a peptide component or peptide payload of the nanocarrier, the number of generations in a branched peptide component of the nanocarrier, the number of amino acids in a generation of a branched peptide component of the nanocarrier, the molecular weight of any component of the nanocarrier, the molecular weight of a peptide component of the nanocarrier, the number of a particular amino acid in a peptide component or peptide payload of the nanocarrier, the number of His residues in a peptide component or peptide payload of the nanocarrier, a score indicative of the hydrophilicity of residues in a peptide component or peptide payload of the nanocarrier, the percentage or proportion of hydrophobic residues in a peptide component or peptide payload of the nanocarrier, the percentage or proportion of hydrophilic residues in a peptide component or peptide payload of the nanocarrier, the presence of a particular type of residues in a particular region of a peptide component or peptide payload of the nanocarrier, the number of polar sidechains of the amino acids of a peptide component or peptide payload of the nanocarrier, the number of ionisable sidechains of the amino acids of a peptide component or peptide payload of the nanocarrier, the absorbance of a peptide component of the nanocarrier at a predetermined wavelength, the net charge of the peptide component of the nanocarrier at a particular pH, the isoelectric point of the peptide component of the nanocarrier, and the isoelectric point of a particular region of the peptide component of the nanocarrier. optionally wherein the input structural properties of the nanocarrier comprise one or more of, or all of the properties listed in Table 1 or Table 2.

9. The method of any preceding claim, wherein the input properties comprise input functional properties and the output properties comprise functional structural properties, and wherein the input functional properties of the nanocarrier comprise or consist of properties that are quantified in vitro and/or wherein the output functional properties of the nanocarrier are properties that are determined in vivo.

10. The method of any preceding claim, wherein the input structural properties of the nanocarrier comprise at least one of: the protein to payload ratio (e.g. N/P ratio), the number of positively charged sidechains in a peptide component of the nanocarrier, the molecular weight of a peptide component, and a value indicative of the hydrophilicity or hydrophobicity of the peptide component, optionally wherein the input structural properties of the nanocarrier include at least the protein to payload ratio (e.g. N/P ratio) or wherein the input structural properties of the nanocarrier comprise at least two of: the protein to payload ratio (e.g. N/P ratio), a charge related metric (e.g. the number of positively charged sidechains in a peptide component of the nanocarrier and/or the total number of histidines in a peptide component of the nanocarrier and/or the total number of charges in a peptide component of the nanocarrier), the molecular weight of a peptide component, the lipid to payload ratio (e.g. L ratio), and a value indicative of the hydrophilicity or hydrophobicity of the peptide component (e.g. the sum of the Hopp-Woods hydrophilicity scores from each residue in the peptide component, the sum of the Hopp-Woods hydrophilicity scores from hydrophobic residues in the peptide component and/or the percentage of hydrophobic residues in the peptide component).

11. The method of any preceding claim, wherein the input structural and/or functional properties of the nanocarrier are normalised values, and/or wherein the output functional and/or structural properties of the nanocarrier are normalised values,
optionally wherein a normalised value is obtained by dividing a determined value by a maximum possible or observed value and/or by subtracting a determined value by a minimum possible or observed value and/or wherein the method further comprises normalising the values of one or more input structural features by dividing a determined value a maximum possible or observed value and/or by subtracting a determined value by a minimum possible or observed value.

12. The method of any preceding claim, wherein the output nanocarrier structural properties are selected from: size of the nanocarrier, polydispersity index of the nanocarrier, and zeta potential of the nanocarrier.

13. The method of any preceding claim, wherein the predicted one or more output functional properties of the nanocarrier further comprise one or more properties selected from: cell-specific payload delivery, tissue specific payload delivery, in vitro cytotoxicity, in vivo cytotoxicity, in vitro immunogenicity, in vivo immunogenicity, nanocarrier temperature dependent structural stability, nanocarrier pH dependent structural stability, nanocarrier pH dependent transfection efficiency, nanocarrier concentration dependent structural stability, nanocarrier structural stability in serum, nanocarrier time dependent structural stability, and nanocarrier pH dependent transfection efficiency.

14. The method of any preceding claim, wherein the machine learning model has been trained using training data comprising the value of the one or more input structural properties of a plurality of nanocarriers and the value of one or more functional properties and optionally one or more output structural properties of said plurality of nanocarriers, optionally wherein the training data comprises data for at least 10, at least 25, at least 50, at least 100 different nanocarriers, or at least 150 different nanocarriers.

15. The method of any preceding claim, wherein the machine learning model has been trained using training data comprising data for a plurality of nanocarriers of the same type as the nanocarrier for which the one or more properties are predicted, optionally wherein the nanocarriers for which the one or more properties are predicted and the plurality of nanocarriers in the training data are lipid nanoparticles, peptide-lipid hybrid nanoparticles or dendrimer peptide-lipid hybrid nanoparticles.

16. The method of any preceding claim, wherein the machine learning has been trained using training data comprising measured transfection efficiency for a plurality of nanocarriers, optionally wherein the transfection efficiency is a normalised transfection efficiency, wherein the transfection efficiency in the training data has been measured using a reporter gene signal, wherein the transfection efficiency in the training data is expressed in fluorescence units associated with expression of a genetic payload encoding a fluorescent protein, wherein the transfection efficiency is normalised using a positive and/or negative control value, wherein the transfection efficiency is an in vitro transfection efficiency, wherein the transfection efficiency is an in vivo transfection efficiency, wherein the transfection efficiency has been measured using one or more cell lines and/or types of cells.

17. The method of claim 16, wherein the machine learning has been trained using training data comprising measured in vitro transfection efficiency for a plurality of nanocarriers, and wherein the predicted transfection efficiency is indicative of in vitro and optionally in vivo transfection efficiency.

18. The method of claim 16 or claim 17, wherein the machine learning has been trained using training data comprising measured transfection efficiency for a plurality of nanocarriers in one or more cell lines, and wherein the predicted transfection efficiency is indicative of transfection efficiency in one or more cell lines comprised in the training data and/or in one or more cell lines not comprised in the training data.

19. The method of any preceding claim, wherein the machine learning model is a non-linear model, optionally wherein the machine learning model is an artificial neural network, a tree-based model or a random forest model; and/or
wherein the machine learning model comprises a plurality of models, wherein each model of the plurality of models has been trained to predict a different set of one or more functional and/or structural properties of a nanocarrier, and/or wherein the machine learning model comprises a model that has been trained to jointly predict a plurality of functional and/or structural properties of a nanocarrier, and/or
wherein the machine learning model comprises an ensemble of models and the one or more functional and/or structural properties of the nanocarrier are obtained by combining the output of the models in the ensemble of models.

20. A method of providing a tool for predicting one or more properties of a nanocarrier, wherein the nanocarrier is a non-viral cell delivery system, the method comprising:
Obtaining a training data set comprising, for each of a plurality of nanocarriers:
experimental data quantifying one or more functional properties of the nanocarrier;
experimental and/or in silico determined values of one or more structural properties of the nanocarrier; and
training a machine learning model to predict the values of one or more functional properties and optionally one or more experimentally determined structural properties of a nanocarrier using input values comprising one or more structural properties of the nanocarrier and/or one or more functional properties of the nanocarrier, optionally comprising at least the in silico determined values of one or more structural properties of the nanocarrier;
wherein structural properties of a nanocarrier characterise physico-chemical properties of the nanocarrier and are independent of the activity of a nanocarrier payload, and wherein the one or more predicted functional properties of the nanocarrier comprise the transfection efficiency.

21. A method of providing a candidate nanocarrier that has one or more desired functional and/or structural properties, the method comprising:
providing a plurality of candidate nanocarriers, wherein the candidate nanocarriers differs from each other in their composition and/or structure;
predicting the value of one or more functional and/or structural properties of the candidate nanocarriers using the method of any of claims 1 to 19; and
selecting a candidate nanocarrier from the plurality of candidate nanocarriers on the basis of the predicted value of the one or more functional and/or structural properties.

22. The method of claim 21, wherein selecting a candidate nanocarrier comprises ranking the plurality of candidate nanocarriers based on at least one of the predicted one or more functional and/or structural properties, optionally wherein the one or more functional and/or structural properties comprise the transfection efficiency, optionally wherein the method comprises ranking the plurality of candidate nanocarriers based on their predicted transfection efficiency.

23. The method of claim 21 or claim 22, wherein selecting a candidate nanocarrier comprises excluding nanocarriers of the plurality of candidate nanocarriers that have a predicted value of one or more functional and/or structural properties that does not meet one or more predetermined criteria, or selecting nanocarriers of the plurality of candidate nanocarriers that have a predicted value of one or more functional and/or structural properties that meet one or more predetermined criteria.

24. The method of any of claims 21 to 23, further comprising formulating and/or experimentally validating and/or further optimizing one or more selected candidate nanocarriers, and/or further comprising preselecting a plurality of candidate nanocarriers based on expert knowledge and/or random modification of previously obtained nanocarriers.

25. A system comprising a processor; and a computer readable medium comprising instructions that, when executed by the processor, cause the processor to perform the steps of the method of any of claims 1 to 24.
